SEQUENCE LISTING (6 sequences)

SEQ ID NO: 1 - DNA, 117 bp, Human betaherpesvirus 5
gacgcagttccacggtctcgcgtatcgcttgtacctccaggaagatttgctgcccgttcatcaacaggttacctgagatgctcaggcccgggatgctcttgggacacagcagcccaa

SEQ ID NO: 2 - DNA, 121 bp, Human betaherpesvirus 5
gcacgcacaaagcggtgtgtcacgccgttctctagcgtgacgctgtatacgtcggcagggtccacggggtactcgccacccggcaccagcagggtggagtagcagaggttgtgagccatga

SEQ ID NO: 3 - DNA, 91 bp, Human betaherpesvirus 5
cccgagtgcccaaagatcacaccctccgaagtgcagccccggccatcgttcccgtttgggatcccctgatccacggcggtgtttccccccg

SEQ ID NO: 4 - DNA, 54 bp, Human betaherpesvirus 5
gtccacgtactcgtaggccgagttcccggcgatgaagatcttgaggctgggaag

SEQ ID NO: 5 - DNA, 54 bp, Human betaherpesvirus 5
tttcgttgtcctcgcccagttgaccgtactgcacgtacgagctgttggcgaaat

SEQ ID NO: 6 - DNA, 78 bp, Human betaherpesvirus 5
tccgttgtagaaaggagaaatgtcaaccacgtcacccgtggaagtggcgaaaaaatgataaggatatttggagcgcgc

(19) United States
(12) Patent Application Publication
(10) Pub. No.: US 2024/0240272 A1
(43) Pub. Date: Jul. 18, 2024

(54) TARGETED MASSIVELY PARALLEL SEQUENCING FOR SCREENING OF GENETIC HEARING LOSS AND CONGENITAL CYTOMEGALOVIRUS-ASSOCIATED HEARING LOSS

(71) Applicant: UNIVERSITY OF IOWA RESEARCH FOUNDATION, Iowa City, IA (US)

(72) Inventors: Ryan K. THORPE, Iowa City, IA (US); Richard J.H. SMITH, Iowa City, IA (US); Hela AZAIEZ, Iowa City, IA (US)

(73) Assignee: UNIVERSITY OF IOWA RESEARCH FOUNDATION, Iowa City, IA (US)

(21) Appl. No.: 18/289,316

(22) PCT Filed: May 9, 2022
(86) PCT No.: PCT/US2022/028361

§ 371 Date: Nov. 2, 2023

Related U.S. Application Data
(60) Provisional application No. 63/186,620, filed on May 10, 2021.

(51) Int. Cl. C12Q 1/70; C12Q 1/6811; C12Q 1/6874; C12Q 1/6883

(52) U.S. Cl. CPC C12Q 1/705; C12Q 1/6874; C12Q 1/6883; C12Q 1/6811; C12Q 2600/156

(57) ABSTRACT

In certain embodiments, the present invention provides the design of a targeted, amplicon-based massively parallel sequencing (MPS) panel focusing on amplifying specific regions of interest associated with genetic hearing loss and infectious diseases associated with hearing loss. The present invention also provides the design of a bioinformatics pipeline capable of performing automated analysis of regions of interest generated by the amplicon-based MPS panel, a targeted gene panel, exome sequencing, or genome sequencing.

### CROSS-REFERENCE TO RELATED APPLICATION

This application claims priority to U.S. Provisional Application No. 63/186,620 that was filed on May 10, 2021. The entire content of the applications referenced above is hereby incorporated by reference herein.

### STATEMENT REGARDING FEDERALLY SPONSORED RESEARCH

This invention was made with government support under DC002842, DC002849, and DC017955 awarded by National Institutes of Health. The government has certain rights in the invention.

### BACKGROUND

Hearing loss is the most common neurosensory deficit, affecting approximately 1 in 500 newborns. Genetic causes are implicated in ˜70% of cases in children, with congenital cytomegalovirus (cCMV) infection comprising an additional 15-20% of cases. Screening to identify hearing loss in newborns is currently accomplished via physiologic testing, such as automated auditory brainstem responses or distortion product otoacoustic emissions. Universal hearing screening has significantly improved the age at which children with severe-to-profound hearing loss are diagnosed. Nonetheless, physiologic screening alone is not informative regarding the etiology of hearing loss and may miss babies with mild or progressive forms of hearing loss. The addition of genetic and cCMV testing as an adjunct to physiologic screening is necessary to further reduce the time to diagnosis and treatment of hearing loss.

Comprehensive massively parallel sequencing (MPS) panels are the current standard of care for the diagnosis of bilateral sensorineural hearing loss in children and adults. These panels can be expensive in terms of library preparation/sequencing costs and interpretation costs, with a typical turnaround time of approximately 6-8 weeks. Since the goal of newborn hearing screening is to obtain a diagnosis inexpensively and quickly, current panels are not practical for universal screening. As an alternative, microarray assays are low in cost with a fast turnaround time, but they offer a sufficient diagnostic yield for the comprehensive evaluation of the thousands of variants that have, to date, been associated with genetic deafness.

### SUMMARY

In one aspect, provided herein is a method of detecting a biomarker in a patient's genome comprising:
(a) obtaining nucleic acid sequence data of the patient's genome,
(b) processing the sequence data to generate a patient dataset for the patient, and
(c) comparing the patient dataset to a biomarker panel, and
(d) generating an automated patient report based on biomarkers that are present in the patient dataset, wherein the patient report identifies one or more biomarkers present in the patient dataset and/or copy-number variants present in the patient dataset, and wherein the one or more biomarkers are genetic variants or genomic target regions associated with nonsyndromic genetic hearing loss, nonsyndromic genetic hearing loss mimics, selected genetic syndromes with hearing loss as a major feature, or infection-induced hearing loss.

In one aspect, provided herein is a method for evaluating hearing loss in a patient, the method comprising:
(a) obtaining nucleic acid sequence data from the patient's genome;
(b) processing the sequence data to generate a patient dataset for the patient; and
(c) comparing the patient dataset to hearing loss biomarkers to identify hearing loss biomarkers in the genomic dataset to create a patient report.

In one aspect, the method further comprises:
(c) processing the patient dataset to identify copy number variants in the genomic dataset.

In one aspect, provided herein is a method for evaluating hearing loss in a patient, the method comprising:
(a) obtaining nucleic acid sequence data from the patient's genome;
(b) processing the sequence data to generate a patient dataset for the patient; and
(c) processing the patient dataset to identify copy number variants in the patient dataset to create a patient report.

In one aspect, provided herein is a method of detecting a biomarker (a genetic variant or genomic target region) associated with nonsyndromic genetic hearing loss, nonsyndromic genetic hearing loss mimics, selected genetic syndromes with hearing loss as a major feature, or infection-induced hearing loss in a patient comprising
(a) analyzing a patient dataset,
(b) comparing the patient dataset to a biomarker panel, and
(c) generating an automated report based on biomarkers that are present in the patient dataset
wherein the comparison identifies biomarkers present in the patient dataset and/or copy-number variants present in the patient dataset, and
wherein the method is fully automated with an anticipated turnaround time after sample collection of two weeks or less.

### BRIEF DESCRIPTION OF DRAWINGS

FIG. 1: Flowchart depicting the population-based strategy for selecting autosomal recessive and dominant, X-linked and mitochondrial variants for inclusion in the newborn genetic hearing screening panel. LP/P=likely pathogenic/pathogenic. DVD=Deafness Variation Database.

FIG. 2: Flow chart of steps in the present method.

FIGS. 3A-3D: Example of automated report output from the bioinformatics pipeline identifying LP and/or P variants within the amplified regions of interest; FIG. 3A shows detection of autosomal recessive hearing loss caused by two single nucleotide variants, 3B shows detection of autosomal dominant hearing loss, 3C shows detection of autosomal recessive hearing loss with one single nucleotide variant and one copy number variant, and 3D shows detection of potentially infectious hearing loss. HGVS=Human Genome Variation Society, VEP=Variant Effect Predictor, QD=QualByDepth score, AF=allele frequency, AC/AN=allele count/allele number, Pop=population (AFR=African/African-American, AMR=Latino/Admixed American, ASJ=Ashkenazi Jewish, EAS=East Asian, FIN=European (Finnish), NFE=European (non-Finnish), SAS=South Asian, OTH=Other), Pred. Cons=predicted consequence, CADD=Combined Annotation Dependent Depletion score, SNV=single nucleotide variant, CNV=copy number variant, HCMV=human cytomegalovirus.

FIG. 4: Coordinates of the target regions within the GRCh37 genome assembly based on the present method of selecting variants associated with hearing loss. chr=chromosome.

FIG. 5: Target regions within the human cytomegalovirus (HCMV) genome (Merlin strain) based on the present method of conserved viral target selection. UL=untranslated long regions.

FIG. 6: Coordinates of the target regions within the GRCh37 genome assembly based on the present method of selecting ethnicity- and gender-defining SNPs for quality control and internal monitoring. SNP=single nucleotide polymorphisms.

FIG. 7: List of variants identified within the target regions by the described methods. These variants are ranked in order of highest to lowest minor allele frequency in the gnomAD database. VEP=Variant Effect Predictor. HGVS=Human Genome Variation Society. “c”=coding DNA reference sequence. “p”=protein reference sequence. MAF=minor allele frequency. \N=protein consequence not applicable (3^{rd} column) or allele not identified in gnomAD (4^{th} column).

### DETAILED DESCRIPTION

The impact of hearing loss on a child's development can be profound. It may affect not only language acquisition but also social development and quality of life. Early detection of congenital hearing loss with targeted intervention significantly reduces these negative impacts. In 1994, the Joint Committee on Infant Hearing (JCIH) published a position statement that endorsed the goal of universal detection of infants with hearing loss and encouraged continuing research and development to improve techniques for detection of and intervention for deafness as early as possible. Today, the crucial role of newborn hearing screening (NBHS) is emphasized by the fact that 43 states and territories of the United States have passed laws mandating NBHS, with the remainder of states having implemented universal NBHS without legislation. Currently, the JCIH recommends universal NBHS by 1 month of age, diagnosis by 3 months of age, and early intervention by 6 months of age to allow optimal outcome for children with hearing loss, if warranted and if desired by the family.

The most recent data show that 98.2% of newborns in the United States receive NBHS. Universal screening has led to a significant reduction in the median age at which newborns who are deaf or hard-of-hearing (DHH) are identified in this country. Although the current universal NBHS has been remarkably successful, as will be detailed below, knowledge gained from the universal NBHS, an improved understanding of the genetics of hearing loss, and an increased recognition of the contribution of congenital cytomegalovirus (cCMV) to childhood deafness have provided an opportunity to improve the current NBHS.

Over the past 20 years, our understanding of genetic deafness has greatly improved. Along with diagnostic audiologic evaluation, diagnostic genetic testing platforms now form a cornerstone for evaluation of DHH newborns and children. An etiological diagnosis is provided in ˜50% of DHH newborns who undergo genetic testing. Genetic testing can also identify mild deafness, later-onset childhood deafness, syndromic forms of deafness, risk factors for aminoglycoside-induced deafness, and auditory neuropathy that may not be detected by the current physiologic NBHS. A genetic test for deafness that could be translated into a universal genetic screening test would form a powerful complement to the current NBHS; however, to date no genetic screening has been incorporated as part of a NBHS program in the United States.

An additional important contributor to childhood deafness is congenital cytomegalovirus infection (cCMV), estimated to be a cause of ˜10% of congenital deafness and 15-20% of childhood deafness. Because cCMV often presents as mild, fluctuating, and progressive deafness, detection with a physiologic screen can be challenging. The incorporation of cCMV screening in a NBHS program would provide etiological information, improve ascertainment, and further complement the physiologic NBHS.

Creating a comprehensive newborn hearing screen that includes physiologic, genetic, and cytomegalovirus screening would have multiple benefits including (1) identifying newborns at risk for hearing loss and who could benefit from early intervention but are missed by the current physiologic screen, (2) providing etiologic information as part of the screen, (3) detecting nonsyndromic hearing loss mimics, (4) possibly decreasing the number of children who are lost to follow up, and (4) potentially saving costs by reducing additional unnecessary testing. This invention aims to provide a conceptual framework for a comprehensive NBHS program that incorporates the current physiologic screening as well as molecular screening that includes genetic screening and cCMV screening. Targeted cCMV screening for those newborns who fail the physiologic screen has been incorporated in several states and thus there is precedent for in inclusion of some molecular screening.

Relevant to modifying the current physiologic NBHS is achieving the goal of universal NBHS, which is to identify ALL newborns with or at risk for permanent hearing loss. Detecting and establishing an etiologic diagnosis for deafness in the newborn period is challenging because of the number of possible causes and the clinical variability in presentation (Table 1).

TABLE 1
Classification of permanent childhood deafness

| Classification | Category |
|---|---|
| Mechanism | Conductive |
| | Sensorineural |
| | Mixed (conductive and sensorineural components) |
| | Auditory neuropathy spectrum disorder |
| Degree | Slight (16-25 dB HL) |
| | Mild (26-40 dB HL) |
| | Moderate (41-55 dB HL) |
| | Moderately severe (56-70 dB HL) |
| | Severe (71-90 dB HL) |
| | Profound (>90 dB HL) |
| Symmetry | Bilateral-symmetric |
| | Bilateral-asymmetric |
| | Unilateral |
| Classification | Category |
| Progression | Stable |
| | Progressive |
| | Fluctuating |
| Onset | Congenital |
| | Prelingual |
| | Postlingual |
| | Childhood |
| Frequency | High frequency (downsloping) |
| pattern of | Low frequency (upsloping) |
| deafness | Mid-frequency (cookie bite) |
| | All frequencies (flat) |
| Associated | Nonsyndromic (no other clinical findings) |
| clinical | Syndromic (other clinical findings) |
| findings | Nonsyndromic mimics (syndromic HL diagnosed |
| | when other clinical findings are not yet apparent) |

HL = hearing loss

Hearing loss is described as slight (16-25 decibel hearing loss, dB HL), mild (26-40 dB HL), moderate (41-55 dB HL), moderately severe (56-70 dB HL), severe (71-90 dB HL), or profound (>90 dB HL); can be unilateral or bilateral; and is either asymmetric or symmetric (Table 1). Select frequencies can be affected that can give an audiogram a specific shape or profile associated (for example, down-sloping if high frequencies are impacted more than low frequencies, or up-sloping if the reverse is true; Table 1). Deafness can also be defined by the site of impairment in the auditory system. A conductive hearing loss (CHL, see a list of definitions in Table 2) implies that transmission of sound through the external ear canal or middle ear is impaired, as in the case of a middle ear effusion, stenosis of the ear canal, or fixation of the ossicular chain. A sensorineural hearing loss (SNHL), in comparison, reflects compromised transmission of the neural signal along the auditory pathway, be it in the cochlea, the auditory nerve, or more proximally in the brainstem and cortex. In the United States and other developed countries, most permanent congenital hearing loss is SNHL and the majority of SNHL (˜70%) is due to a genetic cause. Environmental causes of SNHL, such as infections, hypoxia, and trauma, are a significant but smaller contributor to congenital hearing loss compared with genetic causes.

TABLE 2
Definitions

| | |
|---|---|
| Auditory | An electrical potential measured in response to auditory |
| brainstem | stimulus using electrodes placed on the scalp |
| response | |
| (ABR) | |
| Biomarker | A genetic variant or genetic target region that, when |
| | sequenced, allows for the detection of genetic hearing |
| | loss or infectious hearing loss |
| Amplicon | A fragment of DNA within the genome that is the |
| | product of an amplification process, such as polymerase |
| | chain reaction or targeted amplicon-based massively |
| | parallel sequencing |
| Conductive | Hearing loss due to inability or inefficiency of sound |
| hearing loss | transmission through the external ear canal, across the |
| (CHL) | tympanic membrane and through the ossicles to the inner |
| | ear |
| Massively | High throughput DNA sequencing technology that allows |
| parallel | simultaneous (parallel) sequencing of millions of |
| sequencing | fragments of DNA in short times (also known as next- |
| | generation sequencing) |
| Nonsyndromic | Syndromic forms of deafness that present at birth as |
| deafness | nonsyndromic deafness (no other associated |
| mimics | abnormalities) and therefore may initially clinically |
| | appear to be isolated hearing loss; examples include |
| | but are not limited to Usher syndrome and Jervell |
| | and Lange-Nielsen syndrome |
| Nonsyndromic | Hearing loss that is isolated and not associated with |
| hearing loss | other clinical features |
| (NSHL) | |
| Otoacoustic | A measurement of the response of outer hair cells to |
| emission | sound |
| (OAE) | |
| Pathogenic | A genetic variant with confirmed evidence relating it to a |
| variant | human genetic disease or disorder |
| Selected | Syndromes with hearing loss as a major feature (e.g., |
| syndromes | Waardenburg syndrome, branchiootorenal syndrome) |
| associated with | |
| hearing loss | |
| Sensorineural | Hearing loss due to damage or malfunctioning of the |
| hearing loss | sensory portion of the inner ear, organ of Corti, or |
| (SNHL) | auditory nerve (e.g., DFNB1) |
| Target region | A specific region within a genome that contains a known |
| | pathogenic or likely pathogenic variant, or a well- |
| | conserved region that allows for the detection of |
| | infectious hearing loss |
| Variant | A difference in the human genome from the reference |
| | sequence that may be inherited and may cause disease, |
| | put an individual at risk for disease, or may have no |
| | effect on health. A variant could be a single nucleotide |
| | variant or a copy number variant. |
| Variant of | Agenetic variant with an unclear relation to a human |
| uncertain | genetic disease or disorder |
| significance | |
| (VUS) | |

The ideal NBHS must be inexpensive, fast, easy to learn and administer by screeners, acceptable to the screened individuals, and accurate (high specificity and high sensitivity). Current NBHS programs in the United States use a physiologic evaluation of auditory function in response to sound. Methods used include an otoacoustic emission (OAE) screening test, which measures responses from the outer hair cells of the cochlea; an automated auditory brainstem response (AABR) screening test, which records response to sound based on the neural transmission of a signal from the cochlea to the brainstem; or a combination of both. These screening tests are low cost and can be administered within minutes by a trained screener. As highly validated testing methods, they have been widely adopted. For each ear, the NBHS results in a “pass” (screened negative) or a “refer/did not pass” (i.e., fail, screened positive), which indicates possible hearing loss and the need for further evaluation. For newborns who do not pass the NBHS, diagnostic/confirmatory audiological testing is required to determine the type, degree, and configuration of hearing loss. Further clinical evaluation focuses on determining the degree and cause of hearing loss and is performed in concert between the primary care provider and otolaryngologist. This evaluation varies based on type of hearing loss but by current guidelines include clinical examination and genetic testing in selected clinical scenarios, followed, if necessary, by imaging, laboratory tests, and referrals to specialists. Referrals and follow ups are guided by findings determined by audiometric and physical examination, and frequently include referral to a geneticist and other relevant medical specialists. Typically, the finding of hearing loss is also reported to the state EHDI program. A treatment or habilitation plan is then developed.

Although the NBHS has been remarkably successful, there are several ways in which it could be improved:

### 1) The Current Physiologic NBHS has a Low Positive Predictive Value

Any screening test must weigh false negatives versus false positives to best serve the population screened. “False positive” may refer to the actual screening device used but here we refer to the overall result given to parents—“pass” or “refer”—versus the subsequent diagnostic confirmatory physiologic testing, which serves as the “gold standard.” The positive predictive value (PPV) is the probability that subjects who screen positive truly have the disease. An ideal screening test would have a high PPV and a high sensitivity. While negative predictive value and specificity are also considered in design of screening tests, a confirmatory or diagnostic test should aim for a high negative predictive value and a high specificity as false negatives are reduced.

Data show that of the 3,681,776 newborns documented as undergoing NBHS in 2018, 60,258 (1.6%) did not pass. Of these children, 38,634 went on to have confirmatory audiologic testing. Of these, only 6,432 (16.6%) were found to have deafness on confirmatory diagnostic audiometric testing. Therefore, 83.4% of children who screened positive did not have hearing loss, equating to a PPV of 16.6% for the NBHS. The true sensitivity of this screening program is not known, as there are no large studies that have performed diagnostic testing on all newborns to identify the number of false negative instances (i.e., newborns who passed screening but were found to have hearing loss). From a practical sense, this means that the vast majority of newborns who refer on the physiologic NBHS do not have permanent hearing loss.

While the false positive rate is a concern for the physiologic NBHS, other newborn screening tests have similar performance. For example, a study in California showed that of 755,673 newborns screened for a panel of inborn errors of metabolism using tandem mass spectrometry, 461 newborns screened positive (0.13%). Of the 386 children who underwent confirmatory testing, 335 (86.7%) were found to be false positives and have no inborn errors of metabolism. For this panel, the overall PPV is 13.2%. Other newborn screenings for inborn errors of metabolism have similar false positives rates and positive predictive values.

As a universal screening test performed on a vulnerable population, reducing the false positive rate for the NBHS is important. False positives have the immediate effect of not only unnecessarily worsening parental anxiety and increasing health-care costs, but also lead to a decrease in follow up (discussed in a separate section below). Increasing the PPV should be a goal to improve the current physiologic NBHS but is not an inherent benefit of adding genetic screening.

2) Types of Deafness that are not Identified by the Current NBHS

The effect of mild-to-moderate hearing loss on language development has been heavily debated, but new data and review of previous studies indicate that it is a significant risk factor for communication difficulties. Data from the CDC show that the majority (56.1%) of bilateral hearing loss in newborns is mild-to-moderate, however an even larger percentage of newborns may be affected, as mild deafness can go undetected by NBHS (depending on the thresholds and testing methods that are used). Although implementing physiologic screening at lower hearing levels would detect more mild-to-moderate hearing loss, it would also increase false positive rates and burden confirmatory diagnostic audiology evaluation. The precise sound intensity used in the NBHS is difficult to define precisely because screening equipment manufacturers choose stimulus levels and characteristics for their own equipment that cannot be adjusted by the operator. The screening level of AABR is typically programmed into the screening equipment and is based on research indicating the optimal screening level for AABR. There are no calibration standards for transient stimuli or for determining the actual stimulus levels in ear canals of newborns. Stimulus levels may be greater than specified by the manufacturer because of the small size of a newborn ear canal, thereby leading to false negative results. Identification of infants with mild hearing loss that is permanent and/or progressive is integral to improving the current physiologic NBHS.

Children who develop hearing loss after the newborn period also will not be detected by the current NBHS. This limitation is important as the number of children with significant hearing loss increases throughout childhood from a congenital prevalence of 1.33 per 1,000 births to an estimated prevalence in school-aged children of 2.83 per 1,000. Studies of the prevalence of childhood deafness are primarily based on surveys and although further research in this area is needed, particularly regarding prevalence in preschool-aged children, it is clear that the prevalence of deafness increases with age during childhood. It is important to note that a significant number of children who develop prelingual hearing loss following the newborn period are not detected by the currently implemented NBHS. It is unclear based on current studies whether these children had congenital hearing loss or hearing loss that began soon after the newborn period and further study is needed to clarify this point. However, because these children are not identified by the current physiologic NBHS, the detection of their hearing loss occurs in a haphazard fashion through a patchwork of school-age hearing screening programs that vary significantly by state. Establishing a universal NBHS that is able to identify children with hearing loss that occurs outside the newborn period would prevent a delay in diagnosis and treatment.

Auditory neuropathy spectrum disorder (ANSD) is characterized by absent or severely abnormal inner hair cell (IHC), synaptic, and/or spiral ganglion function as measured by ABR testing. Outer hair cell (OHC) function is preserved as measured by OAE testing. Therefore, a newborn with ANSD may go undetected by OAE screening when OAE screening is performed alone. Further compounding the challenge presented by ANSD is the fact that it is highly variable, with some infants and children with ANSD having asymmetric or unilateral deafness. At older ages, ANSD is accompanied by poor speech discrimination and poor word understanding, especially in the presence of noise. The prevalence of ANSD is reported to be 2.7% of DHH newborns identified by NBHS programs based on data from the CDC. Other data indicate the prevalence of ANSD to be 1.2%, 5.1%, or 8.4% depending on the population. Forty percent (40%) of ANSD is estimated to have a genetic basis, with the remainder due to acquired causes like hypoxia, prematurity, and jaundice, which explains the increased rate of ANSD in neonatal intensive care units. The list of causative genes for ANSD includes DIAPH3, OTOF, PJVK, and mitochondrial DNA (mtDNA) variants (m.1095T>C) for nonsyndromic ANSD, and AIFM1, DDDP, MPZ, OPA1, PMP22, and TMEM126A for syndromic ANSD, although based on prevalence data there are likely other genes involved. The gene most frequently implicated in nonsyndromic ANSD is OTOF, which is estimated to be responsible for 0.5-3.5% of prelingual deafness across multiethnic cohorts. NBHS that relies only on OAE will not identify these babies, but fortunately the majority of neonatal intensive care units (where the rate of ANSD is highest) perform screening with AABR, as recommended by JCIH, and half of all NBHS is now performed with AABR. The actual number of newborns missed by the current NBHS due to ANSD is not known but ensuring these babies are screened is key to improving screening outcomes.

The current NBHS does not screen for a relatively common risk factor that can cause hearing loss in the newborn period. Several mitochondrial DNA variants lead to exquisite sensitivity to aminoglycoside-induced deafness. Aminoglycosides, in particular gentamicin, are commonly used in the neonatal period due to low cost and effectiveness against Gram-negative bacteria. Newborns who carry certain genetic variants in the mitochondrial gene MT-RNR1 can experience significant deafness with a single dose of these commonly used antibiotics. In addition, animal studies show that there is a synergistic effect of these genetic variants, aminoglycosides, and noise, which further predisposes these newborns to deafness. Prevalence of these mitochondrial variants was found to be 0.2% in one study of 703 children from a neonatal intensive care unit in the United States and 0.19% in a study of 58,397 Chinese children. Identifying these newborns with screening could have immediate treatment implications. In addition, diagnosis could prevent aminoglycoside-induced later-onset hearing loss in vulnerable individuals and lead to targeted cost-effective evaluation in maternal relatives after positive screening results from one individual.

### 3) Limited Etiological Information is Included in the Current NBHS

The current NBHS provides a simple result: “pass” or “refer.” Subsequent audiologic and diagnostic evaluation provides information on degree of hearing loss and etiology. An important goal of diagnostic testing for deafness in children is to identify etiologies that require further diagnostic and treatment implications. Examples include Usher syndrome (deafness-blindness), Pendred syndrome (deafness including inner ear malformations and thyroid abnormalities), and Jervell and Lange-Nielsen syndrome (deafness and cardiac arrhythmias). These syndromic forms of deafness are referred to as “nonsyndromic hearing loss (NSHL) mimics” because they present at birth as nonsyndromic deafness (with no other associated abnormalities readily appreciable on physical examination). Recent data show that these syndromes are more common than previously reported. In a cohort of 2460 individuals of all ages with deafness, the diagnoses of NSHL mimics totaled 25% of all diagnoses and most commonly included Usher syndrome (10%), Pendred syndrome (5%), and deafness-infertility syndrome (4%). Other syndromes with deafness as a major feature include branchiootorenal syndrome (BOR, branchial cleft anomalies, deafness, and renal abnormalities), Waardenburg syndrome (deafness, pigmentation abnormalities, with or without eye abnormalities), and Alport syndrome (deafness, renal disease, and eye abnormalities). Early genetic diagnosis of syndromic forms of deafness would significantly reduce other testing and provide opportunities for early intervention. Identification of a genetic etiology of hearing loss informs patients of the pathogenesis, sequelae (if any), and expected course of their condition and also refines estimates of recurrence risk for family members. The information may influence family reproductive and financial planning.

### 4) The Current NBHS Lacks Modularity and Prognostic Ability

The classification of hearing loss continually evolves as anomalies in the auditory pathway are studied in closer detail. Not only is the location within the pathway important (presynaptic, synaptic, and postsynaptic), each partition has associated structures that may be individually affected. Within the organ of Corti, where sound waves are converted into neural impulses, specific genes have been identified that are uniquely characteristic of the inner hair cells, outer hair cells, and supporting cells, respectively. Several genes have been implicated in maintenance of the unique properties of the tectorial membrane, which is required for fluid coupling and sound frequency selectivity within the inner ear. Still more genes have been implicated in the function of the auditory synapse and the spiral ganglion cells themselves each of these examples informs an ever-expanding body of evidence regarding predicted outcomes of cochlear implantation and other forms of habilitation. A specific genetic diagnosis allows patients to benefit from this knowledge, past, present, and future. Furthermore, as new genes and variants within those genes are identified as causative for hearing loss, a genetic panel is easily modifiable; physiologic screening, in contrast, would require entirely new technologies to improve diagnostic granularity.

### 5) A Significant Number of Newborns are Lost to Follow Up in the Current NBHS

A significant number of newborns are lost to diagnostic follow up in the current NBHS protocol. The data from 2015 showed that 27.9% of newborns who referred on NBHS were lost to follow up prior to diagnostic audiology. Of these newborns, 54.7% were lost to follow up for unknown reasons; 14.7% were cases in which there was an inability to contact the newborn's guardian; and in the remaining 30.6% of cases, the parents or family were contacted but were not responsive to follow-up requests. While there are several reasons that children are lost to follow up including social, economic, and geographic factors, data show that altering testing methodologies, including a screen-rescreen policy as well as increasing time from birth can improve positive predictive value. It is not unreasonable to assume that by providing more etiologic information to parents during the screening process and improving the positive predictive value of screening, fewer children would be lost to follow up. Providing an etiologic diagnosis sooner may motivate parents to seek early intervention services.

6) Screening for cCMV-Related Deafness

The leading nongenetic cause of congenital hearing loss is congenital cytomegalovirus infection (cCMV), which is estimated to underlie 15-20% of all childhood deafness. Of newborns who refer on physiologic NBHS, ˜6.0% will test positive for cCMV. cCMV infection occurs in 0.64% (˜1 in 200) of all live births in the United States and can lead to permanent disability, including cognitive impairment, cerebral palsy, developmental delay, and hearing and vision loss. Risk for cCMV infection is 32% following a primary maternal infection during pregnancy, however in CMV-positive mothers, the risk of maternal-to-fetal transmission is much lower and is estimated at only ˜1.4%. About 10% of cCMV-infected babies are obviously symptomatic at birth with signs and symptoms of infection that include intrauterine growth restriction, microcephaly, and jaundice (symptomatic infection); SNHL is present in approximately 30% of this cohort. The remainder of babies born with cCMV display no obvious outward signs of infection and are classified as having asymptomatic CMV. Approximately 14% of these babies develop SNHL by 5 years old, with 25% developing SNHL by age 18; however, the risk of developing SNHL beyond 5 years old is not statistically different between cCMV-positive and cCMV-negative groups.

The defining characteristic of hearing loss due to cCMV is its variability. In children born with asymptomatic cCMV, it is most frequently mild, unilateral, fluctuating, and progressive. In children born with symptomatic cCMV, hearing loss is more likely to be bilateral and moderate to severe/profound in degree but is still frequently progressive and fluctuating. Presumably, it is for these reasons that in a recent study of 99,945 infants screened for cCMV, 43% of infants with cCMV and deafness at birth were not identified by NBHS. As a step toward better cCMV detection, selected hospitals with birthing centers, as well as several states, now implement targeted cCMV screening programs for infants who refer on NBHS. Although these programs have been successful, they are not universal and only identify newborns who refer on NBHS and are then tested for cCMV. One recent study of 10,964 newborns found that in a targeted saliva-based cCMV screen of the 171 newborns who referred on the physiologic NBHS, only 3 screened positive for cCMV. Another recent study performed universal cCMV screening in 1716 newborns using quantitative real-time PCR on dried blood spots and detected 3 positive cases who all passed physiologic NBHS. While saliva-based tests are more sensitive that blood spots for cCMV detection, additional sample collection and processing is required.

Detection of neonates with cCMV-positive hearing loss is important because antiviral therapy can improve or halt SNHL in symptomatic cCVM-positive babies. A recent review also suggests that antiviral treatment in newborns with asymptomatic cCMV and hearing loss can prevent progression of deafness, however this study was retrospective and lacked long-term follow up. More definitive prospective studies on asymptomatic cCMV are ongoing.

cCMV detection can be incorporated into the NBHS by providing either universal screening or targeted screening after a failed NBHS. While targeted screening for cCMV is less costly, as noted earlier a significant number of cCMV-positive children will not be identified. Universal screening for cCMV would identify these newborns and be a valuable addition to a comprehensive NBHS. In particular, the prospect of identifying and treating hearing loss in children with asymptomatic cCMV makes screening for cCMV of added importance.

### APPLICATION BEYOND NEONATES

The prevalence of hearing loss increases with age, more than doubling by the time a newborn reaches primary school. While some portion of this increase is explained by cases of acquired hearing loss, there is a cumulative addition by way of progressive genetic hearing loss or hearing loss that is not severe enough to be picked up by newborn screening. This hearing loss may manifest in infancy, adolescence, young adulthood, or older. By the age of 85, most people will have hearing loss that affects their ability to communicate with others. Adults and children alike with hearing loss are at risk of societal discrimination, depression, and difficulty with educational attainment. Individuals older than 65 with hearing loss are also more likely to develop dementia, with a more rapid decline in cognition compared to their normal hearing peers. Studies of monozygotic versus dizygotic adult twins have confirmed that genetic factors are a significant contributor to adult-onset hearing loss.

While the diagnostic yield of comprehensive genetic testing for hearing loss is lower for adult-onset hearing loss than congenital hearing loss, these panels remain the next best step in the evaluation of an adult with bilateral SNHL after physical examination and audiometry. As with genetic testing in children, receiving a diagnosis in adults provides information regarding etiology, sequelae, and predicted clinical course. Among deaf adults, an inexpensive, rapid test would improve access to definitive genetic diagnosis, and would inform the natural history of their hearing loss, their treatment options, and future reproductive planning. A panel that allows access to low cost, high throughput analysis is thus of equal importance in adults as in children.

### Methods of Screening

In certain aspects, the present invention provides the generation of ethnically inclusive targeted amplicon-based MPS panels. The method balances sequencing costs with diagnostic yield. This panel also enables the detection of cCMV from the same sample in a newborn. This methodology can be applied as an initial low-cost step in the diagnosis of all patients with suspected genetic hearing loss. Through careful design of the panel and its bioinformatics pipeline, the present method can provide a turnaround time of under two weeks from the time of sample receipt.

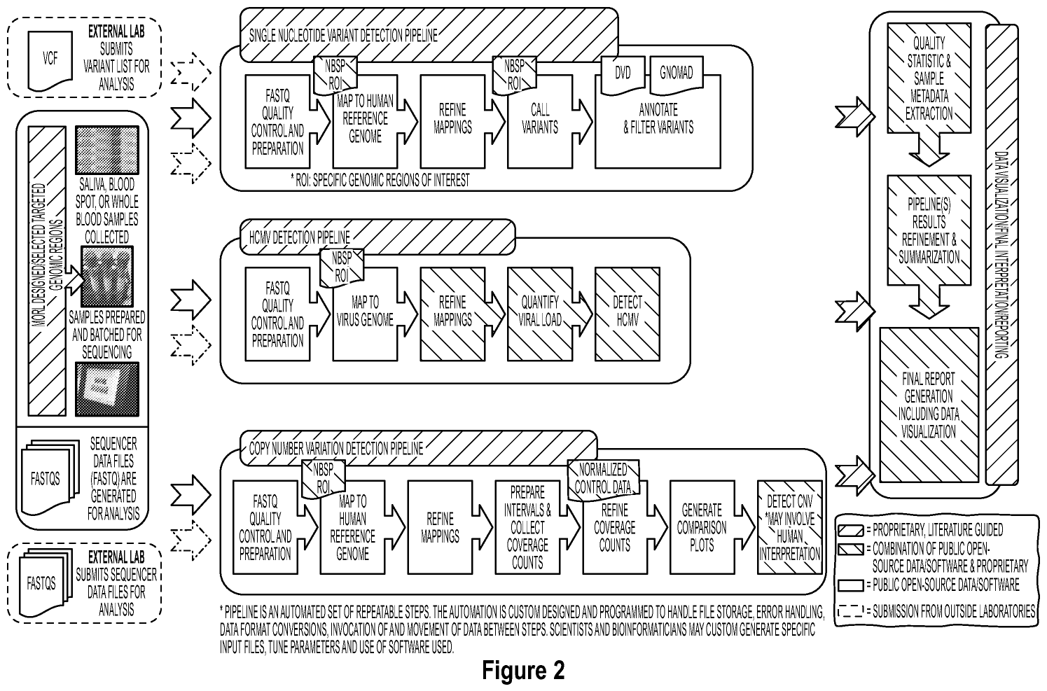

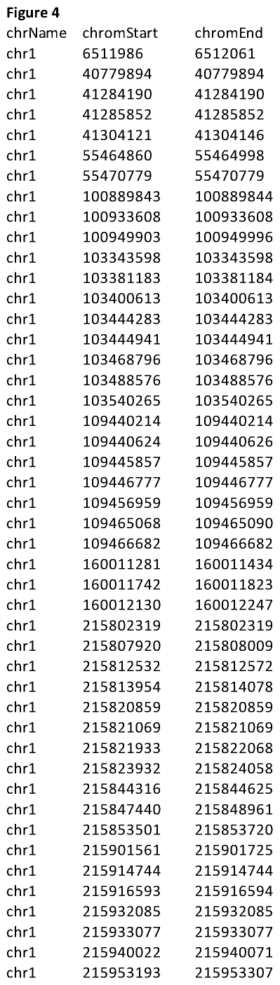

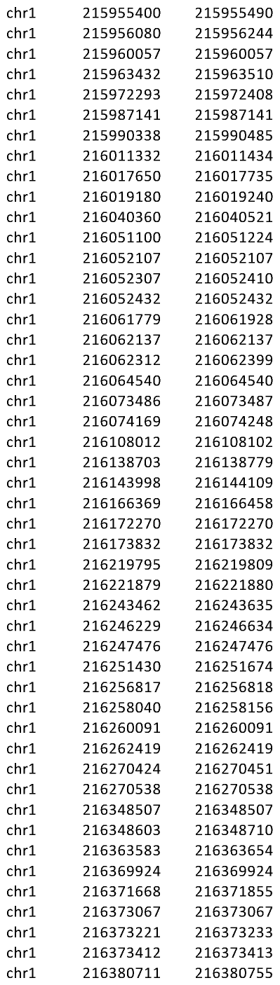

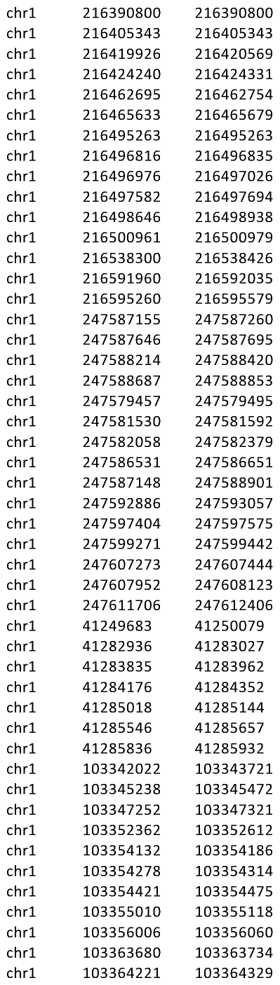

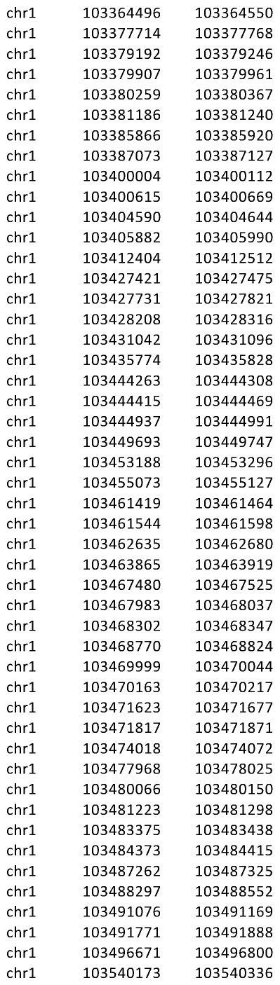

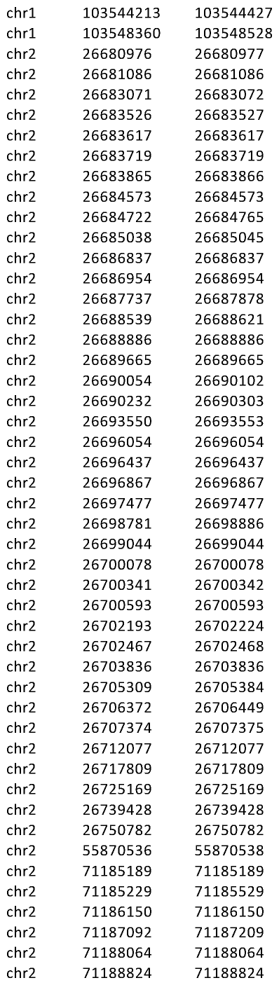

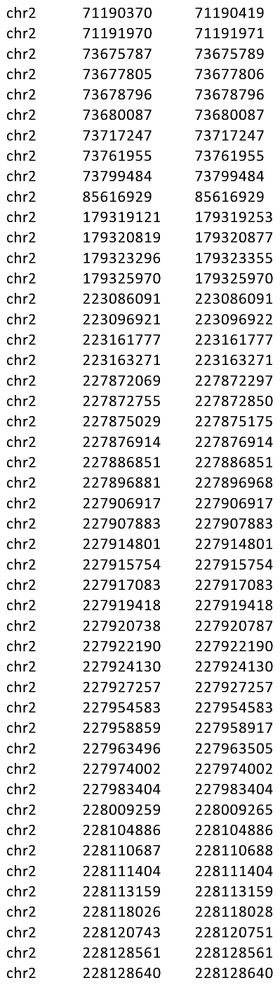

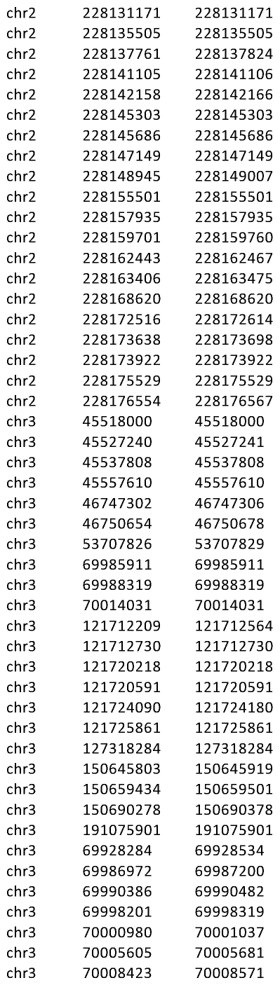

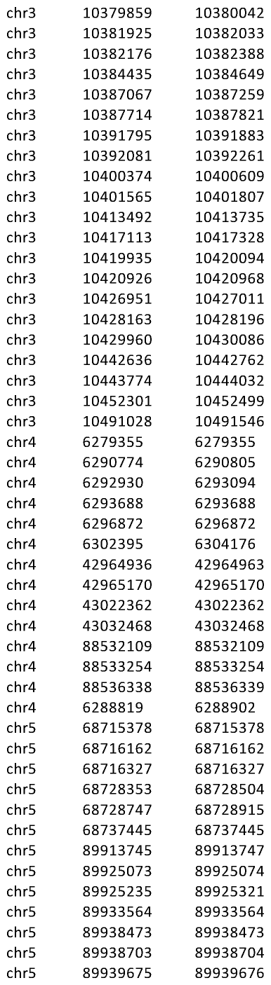

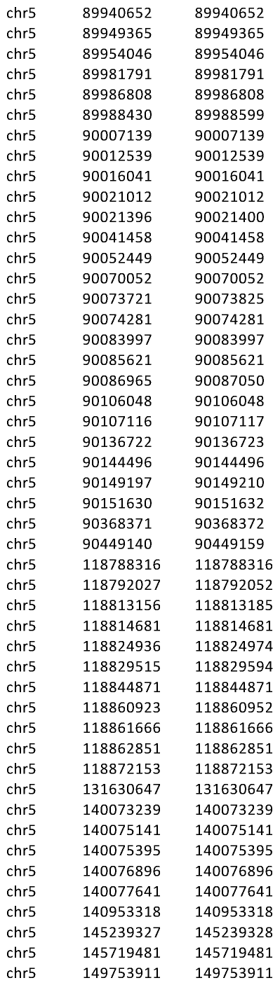

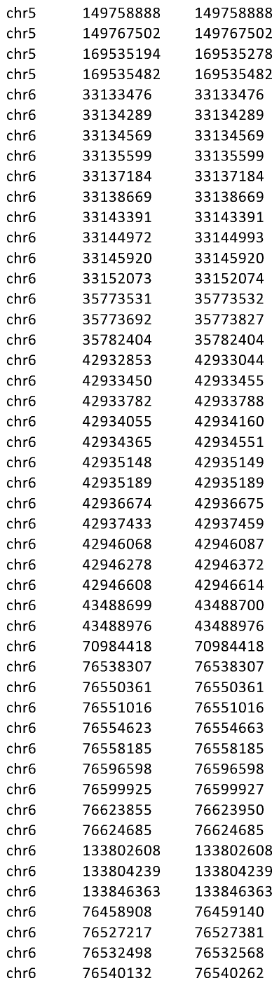

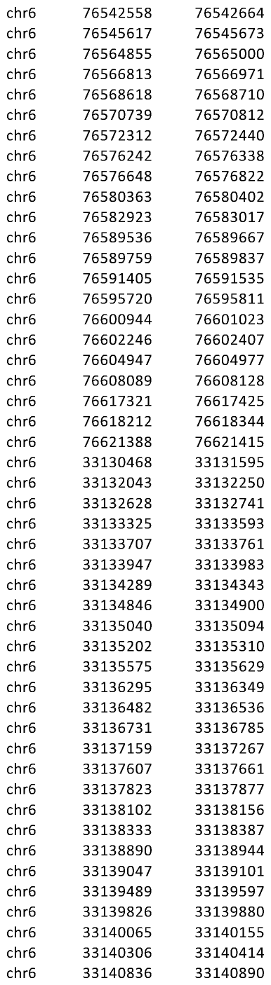

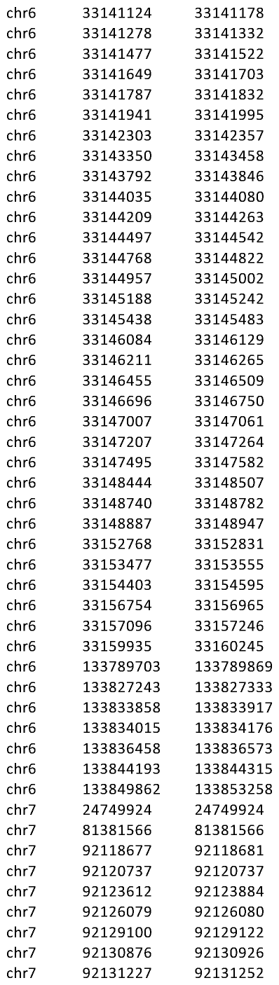

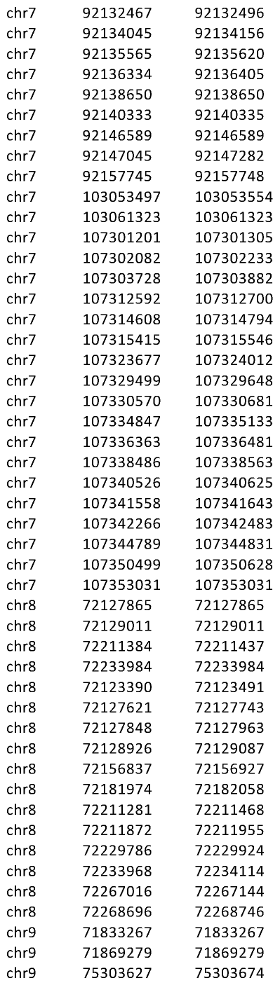

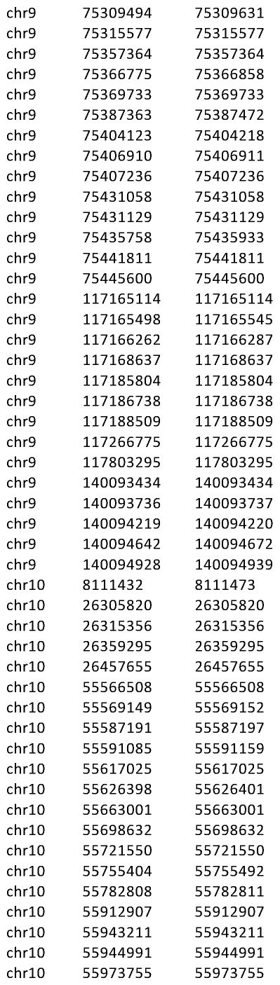

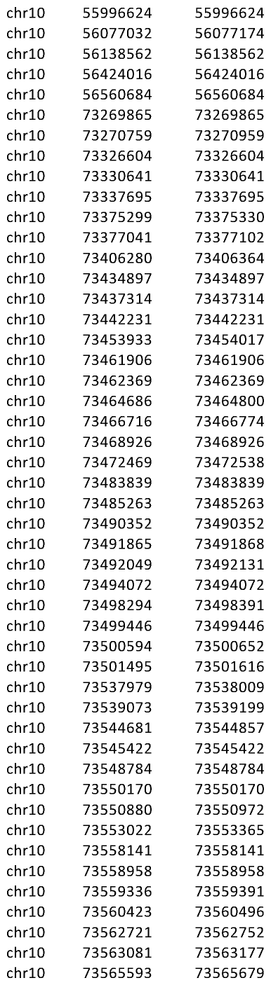

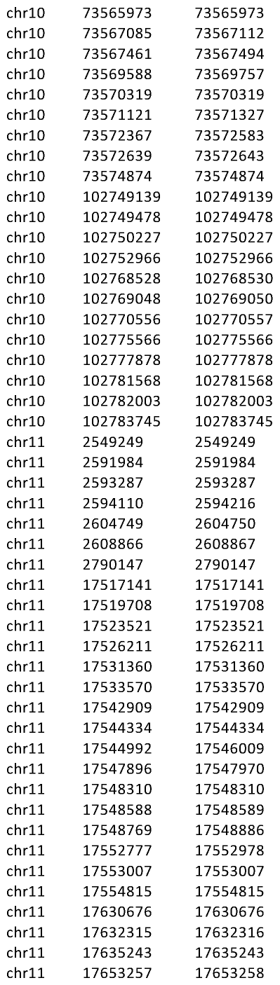

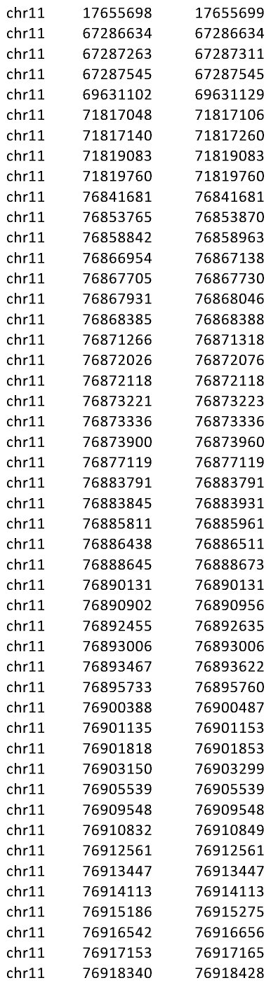

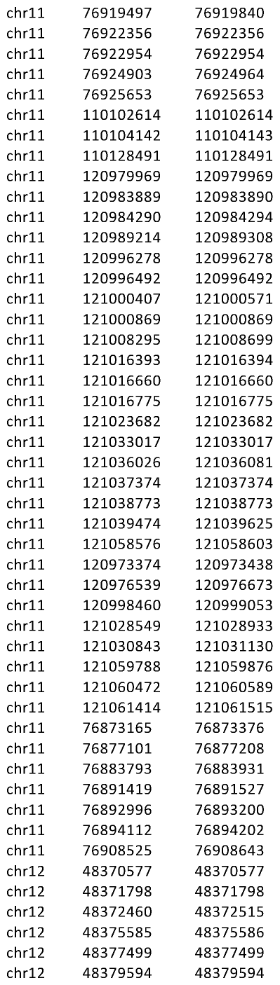

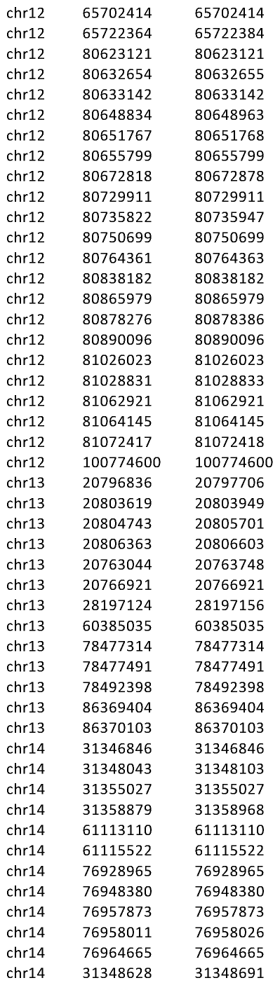

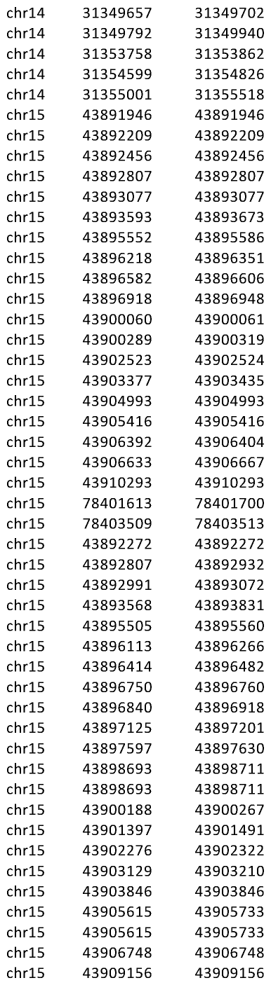

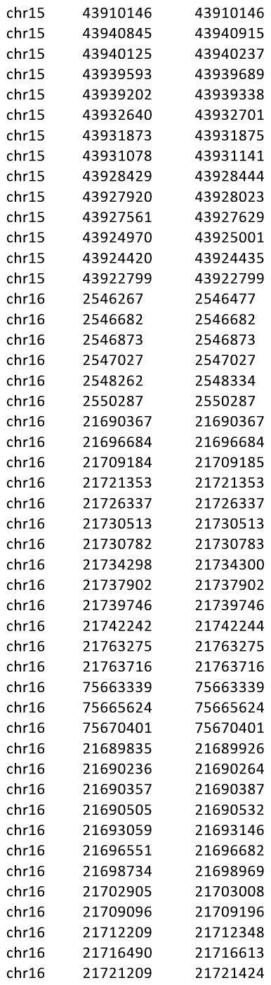

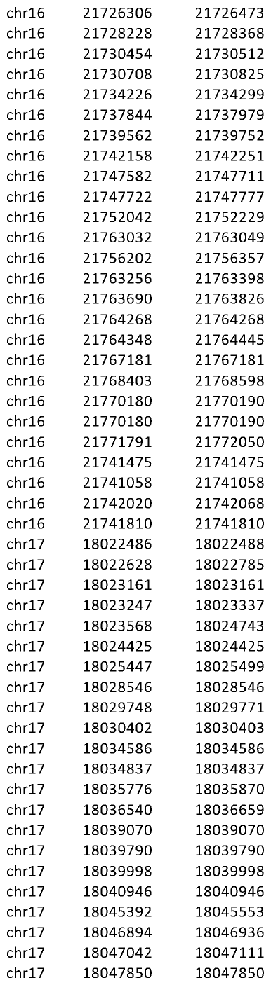

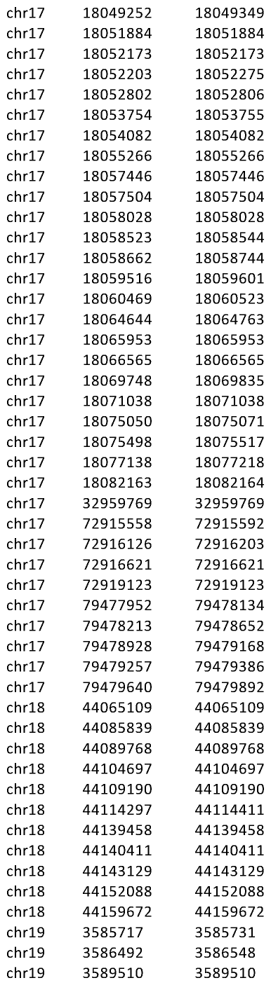

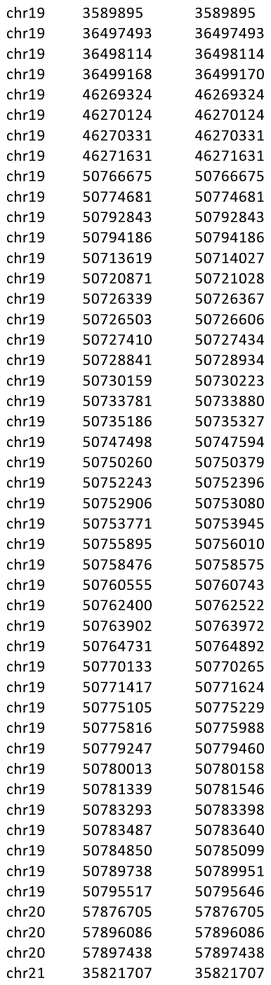

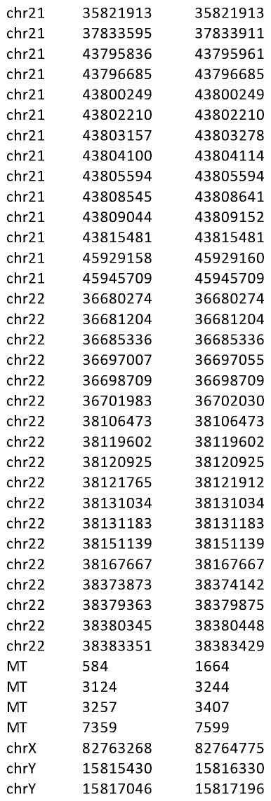

In certain aspects, the method of the present invention involves the following steps (FIG. 2). First, a sample is obtained from a patient. Exemplary samples include buccal swabs, saliva, dried blood spots, and whole blood samples. After DNA extraction and purification, sequencing of targeted regions of the genome is performed. Sequencing methodology includes genome sequencing, exome sequencing, targeted amplicon-based library preparation, or deafness-specific targeted gene panels (such as OtoSCOPE™). From this sequencing data, FASTQ files are generated, which represent the “patient genetic dataset” or “patient dataset.” The patient dataset is then compared to the set of manually curated genetic variants, wherein the comparison identifies single nucleotide variants and/or copy number variants present in the patient dataset (FIG. 4). In certain aspects, the analysis portion of this method is performed in the timespan of hours. In certain aspects, the method is fully automated. In certain aspects, the comparison is performed by a computer.

In one aspect, provided herein is a method of detecting a biomarker in a patient's genome comprising:
(a) obtaining nucleic acid sequence data of the patient's genome,
(b) processing the sequence data to generate a patient dataset for the patient, and
(c) comparing the patient dataset to a biomarker panel, and
(d) generating an automated patient report based on biomarkers that are present in the patient dataset, wherein the patient report identifies one or more biomarkers present in the patient dataset and/or copy-number variants present in the patient dataset, and wherein the one or more biomarkers are genetic variants or genomic target regions associated with nonsyndromic genetic hearing loss, nonsyndromic genetic hearing loss mimics, selected genetic syndromes with hearing loss as a major feature, or infection-induced hearing loss.

In one aspect, provided herein is a method for evaluating hearing loss in a patient, the method comprising:
(a) obtaining nucleic acid sequence data from the patient's genome;
(b) processing the sequence data to generate a patient dataset for the patient; and
(c) comparing the patient dataset to hearing loss biomarkers to identify hearing loss biomarkers in the genomic dataset to create a patient report.

In one aspect, the method further comprises:
(c) processing the patient dataset to identify copy number variants in the genomic dataset.

In one aspect, provided herein is a method for evaluating hearing loss in a patient, the method comprising:
(a) obtaining nucleic acid sequence data from the patient's genome;
(b) processing the sequence data to generate a patient dataset for the patient; and
(c) processing the patient dataset to identify copy number variants in the patient dataset to create a patient report.

In one aspect, provided herein is a method of detecting a biomarker (a genetic variant or genomic target region) associated with nonsyndromic genetic hearing loss, nonsyndromic genetic hearing loss mimics, selected genetic syndromes with hearing loss as a major feature, or infection-induced hearing loss in a patient comprising:
(a) analyzing a patient dataset,
(b) comparing the patient dataset to a biomarker panel, and
(c) generating an automated report based on biomarkers that are present in the patient dataset wherein the comparison identifies biomarkers present in the patient dataset and/or copy-number variants present in the patient dataset, and wherein the method is fully automated and the analysis portion is performed in the timespan of hours.

In certain aspects, the patient genetic dataset is a Massively Parallel Sequence (MPS) FASTQ file.

In certain aspects, the MPS FASTQ file is generated from a genome sequence, an exome sequence, a targeted amplicon-based library preparation, or a deafness-specific targeted gene panel (such as OtoSCOPE™).

In certain aspects, the patient datasets are sequencing data generated from a panel of amplicons.

In certain aspects, the comparison identifies copy number variants present in the patient dataset.

In certain aspects, the biomarker panel comprises at least 96 biomarkers associated with nonsyndromic hearing loss, nonsyndromic hearing loss mimics, and selected syndromes with hearing loss as a major feature. Because a genetic dataset could contain any number of variations within the amplified regions, the biomarker panel comprises a theoretically infinite number of biomarkers. The current panel tests for over 4,000 known LP/P variants.

In certain aspects, the biomarkers associated with nonsyndromic hearing loss, nonsyndromic hearing loss mimics, and selected syndromes with deafness as a major feature are identified from a group of over 200 genes associated with hearing loss.

In certain aspects, the biomarker panel comprises regions within or surrounding GJB2, STRC, STRCP1, CATSPER2, OTOA, and OTOAP1, which allows for the detection of copy number variants associated with non-syndromic deafness and deafness-infertility syndrome.

In certain aspects, the biomarker panel comprises biomarkers designed to detect copy number variants associated with nonsyndromic hearing loss and deafness infertility syndrome.

In certain aspects, copy number variants associated with hearing loss are selected from the group consisting of at least 170 target regions comprising 150-200 base pairs each (FIG. 4).

In certain aspects, the biomarker panel comprises biomarkers associated with infection-induced hearing loss.

In certain aspects, the infection-induced hearing loss is cytomegalovirus-induced hearing loss.

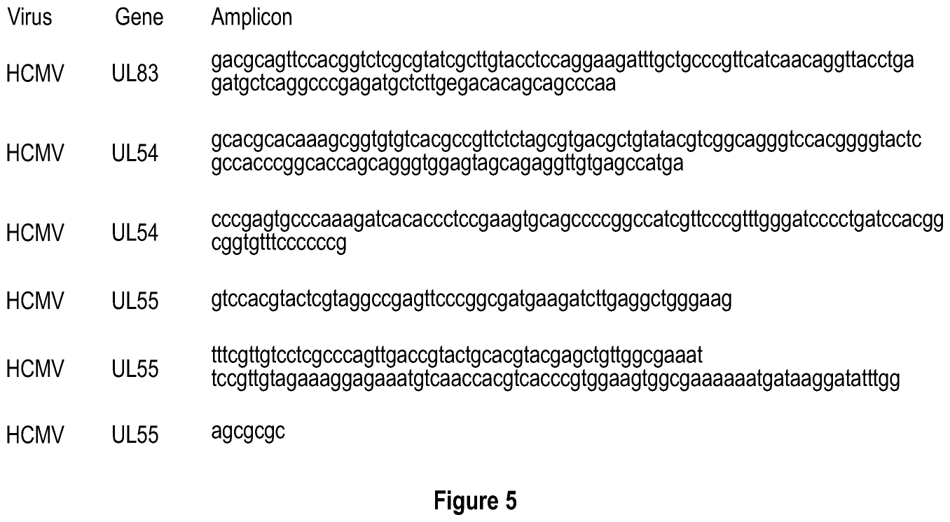

In certain aspects, the biomarkers associated with infection-induced hearing loss are selected from the group consisting of at least six amplicons within highly conserved regions of viral genomes (FIG. 5).

In certain aspects, the analysis method is performed in about the timespan of hours.

The invention will now be illustrated by the following non-limiting Example.

### Example 1

### Amplicon-Based Massively Parallel Sequencing for Newborn Hearing Screening

Hearing loss is common in children, with approximately 1 in 500 newborns being affected. In developed countries, ˜70% of cases are linked to genetic causes. Congenital cytomegalovirus (cCMV) is the second most common cause of hearing loss in newborns, comprising an additional 15-20% of cases. Current screening methods for hearing loss in newborns depend on physiologic measures of hearing; despite the success of this strategy, many newborns who are born with hearing loss remain undiagnosed until childhood or later. Children born with mild-to-moderate hearing are diagnosed later than those with severe-to-profound hearing loss. Furthermore, some forms of genetic hearing loss are progressive and because they may begin as mild or even normal hearing, these children will be under diagnosed or missed completely by the current screening methods.

To improve the yield of testing in newborns, several methods have been proposed. Universal newborn genetic hearing screening using microarrays is currently utilized in China and tests for the most common variants associated with deafness. Though microarrays analysis in inexpensive to perform and straightforward to interpret, the diagnostic yield is poor when compared to comprehensive genetic testing via massively parallel sequencing (MPS). A suitable genetic screening technology for an ethnically diverse population as in the United States has yet to be incorporated on a large scale, and no method has combined genetic testing with cCMV testing. With over 8,000 deafness-causing genetic variations classified as likely pathogenic or pathogenic, an optimal screening platform must be capable of testing for a large number of these variants. Furthermore, copy number variants (CNVs)—large insertions, deletions or conversions in the genome—are implicated in about ˜20% of diagnoses, making them a common contributor to inherited hearing loss and mandating their detection on screening platforms. While microarrays are capable of detecting CNVs via comparative genomic hybridization or single nucleotide polymorphism arrays, the diagnostic yield is low and the false-negative and false-positive rates are high. MPS by comparison is a high-yield diagnostic method for the detection of CNVs.

For cCMV, a sequential diagnostic protocol utilizing polymerase chain reaction (PCR) to test for cCMV for children with abnormal hearing screenings was developed. Though PCR provides excellent sensitivity and specificity for cCMV, MPS has also been shown to be a promising diagnostic modality for not only human cytomegalovirus (HCMV) detection, but also viral load quantification. Therefore, our goal was to combine genetic and HCMV testing into a single MPS platform to create a comprehensive, cost-effective, and high throughput adjunct to physiologic hearing screening in the newborn requiring only a single sample from each baby.

### Amplicon Design

A custom amplicon panel was prepared for multiplexed pooled sample library preparation. The panel was designed based on the human genome reference build GRCh37 (hg19) from the Genome Reference Consortium. Amplicons were designed based on submission of the panel's target regions using the D3™ Assay Design website. For the detection of cCMV, six amplicons from the HCMV genome were dedicated to amplifying highly conserved regions that are sensitive for diagnosis within the UL54, UL55, and UL83 regions of the HCMV genome (FIG. 5).

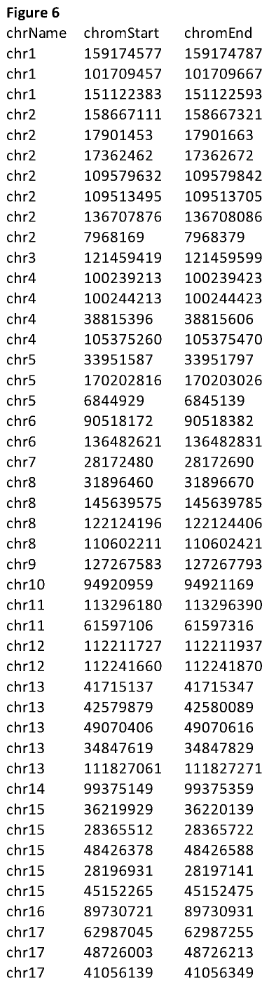

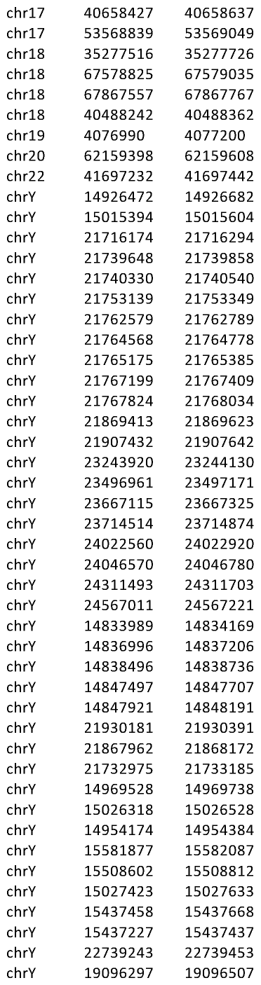

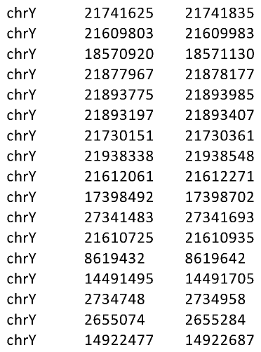

85 target regions were dedicated to ancestry-informative single nucleotide polymorphisms (AISNPs), based on 55 AISNPs (FIG. 6) described by Kidd and colleagues (Kidd K K, Speed W C, Pakstis A J, et al. Progress toward an efficient panel of SNPs for ancestry inference. Forensic Sci Int Genet. 2014; 10:23-32. doi: 10.1016/j.fsigen.2014.01.002) and 30 commonly described AISNPs (FIG. 6) within the Y chromosome (Butler J M. Recent Developments in Y-Short Tandem Repeat and Y-Single Nucleotide Polymorphism Analysis. Forensic Sci Rev. 2003; 15(2):91-111).

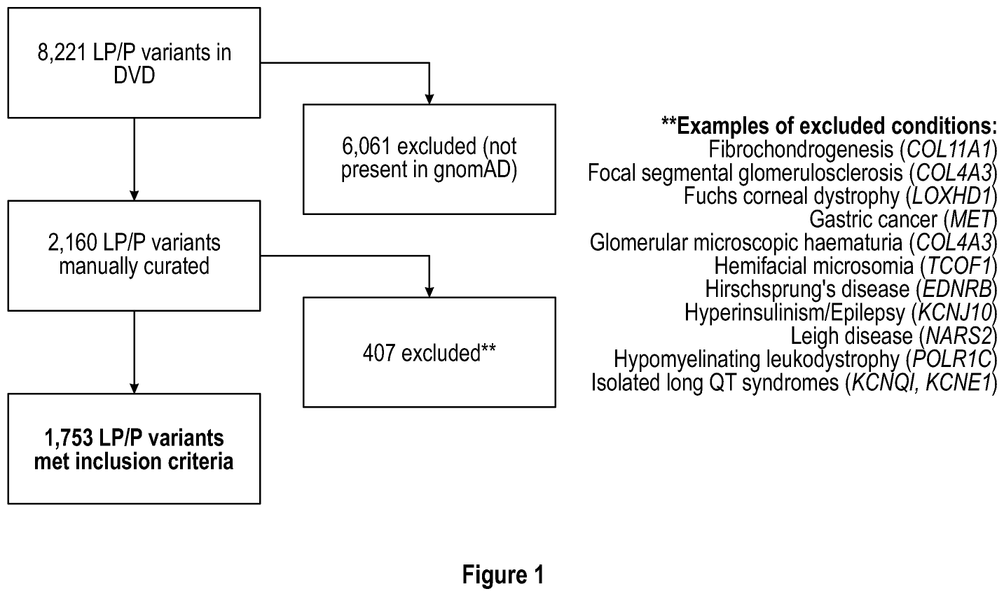

To select single nucleotide variations (SNVs) and small insertion or deletions for autosomal recessive variants within the panel, a population-based approach was used (FIG. 1). The Deafness Variation Database v8.2.1 (Deafness Variation Database. Molecular Otolaryngology & Renal Research Lab at the University of Iowa. deafnessvariationdatabase.org, Published 2020), a curated database of all known variants within genes associated with hearing loss, was queried for all known likely pathogenic and pathogenic variants, yielding 8,221 variants. From this list, only variants that appeared at least once in gnomAD (Karczewski K J, Francioli L C, Tiao G, et al. The mutational constraint spectrum quantified from variation in 141,456 humans. Nature. 2020; 581(7809):434-443. doi:10.1038/s41586-020-2308-7), a collection of exomes or genomes from 141,456 individuals, were included. This list was manually curated by experts in the field of genetic hearing loss to limit the panel to variants associated with nonsyndromic hearing loss, nonsyndromic hearing loss mimics, and selected syndromes associated with deafness. Deafness-causing autosomal dominant mutations are most frequently novel missense mutations. Thus, a strategy involving covering entire exons in these genes was pursued. The exons were selected based on gene-specific structural analysis. Untranslated exons were excluded. A full list of included exons is shown in Table 3.

TABLE 3
Table 3: Genes associated with autosomal dominant hearing loss included
on our panel. The exons covered in each gene are listed.

| Gene | NM transcript | Exons Covered |
|---|---|---|
| NLRP3 | NM_183395.2 | 1-11 |
| TECTA | NM_005422.2 | 3-23 |
| WFS1 | NM_006005.3 | 2-8 |
| MYO6 | NM_004999.3 | 1-35 |
| KCNQ4 | NM_004700.3 | 1-7 |
| MITF | NM_198159.2 | 1-10 |
| MYO7A | NM_000260.3 | 2-48 |
| COL11A1 | NM_080629.2 | 1-66 |
| COL11A2 | NM_080680.2 | 1-66 |
| EYA1 | NM_000503.5 | 2-17 |
| EYA4 | NM_004100.4 | 11-20 |
| ACTG1 | NM_001199954.1 | 2-6 |
| ATP2B2 | NM_001001331.2 | 2-22 |
| COCH | NM_001135058.1 | 4-12 |
| MYH14 | NM_001145809.1 | 2-37 |
| SOX10 | NM_006941.3 | 3-5 |
| PAX3 | NM_181459.3 | 1-9 |
| DFNA5* | NM_004403.2 | 6-7 |

*= introns 7 and 8 were also covered in DFNA5.

For the detection of CNVs, the panel focuses on regions within and surrounding GJB2, STRC, STRCP1, CATSPER2, OTOA, and OTOAP1, which comprise the majority of all CNVs associated with non-syndromic deafness and deafness-infertility syndrome. A list of amplicons covered within these regions is included in FIG. 4.

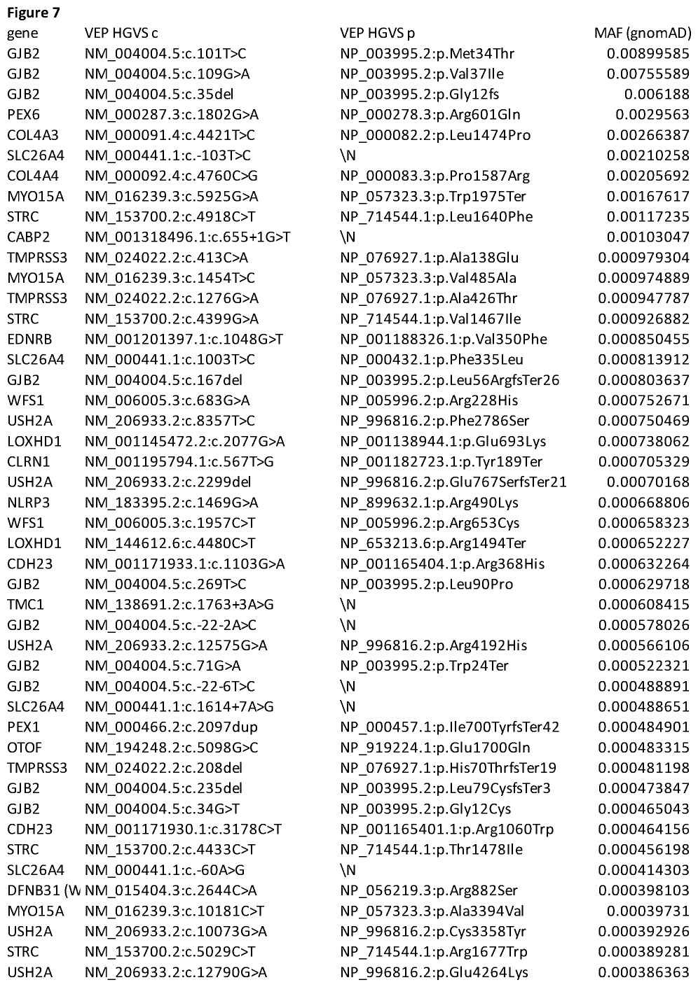

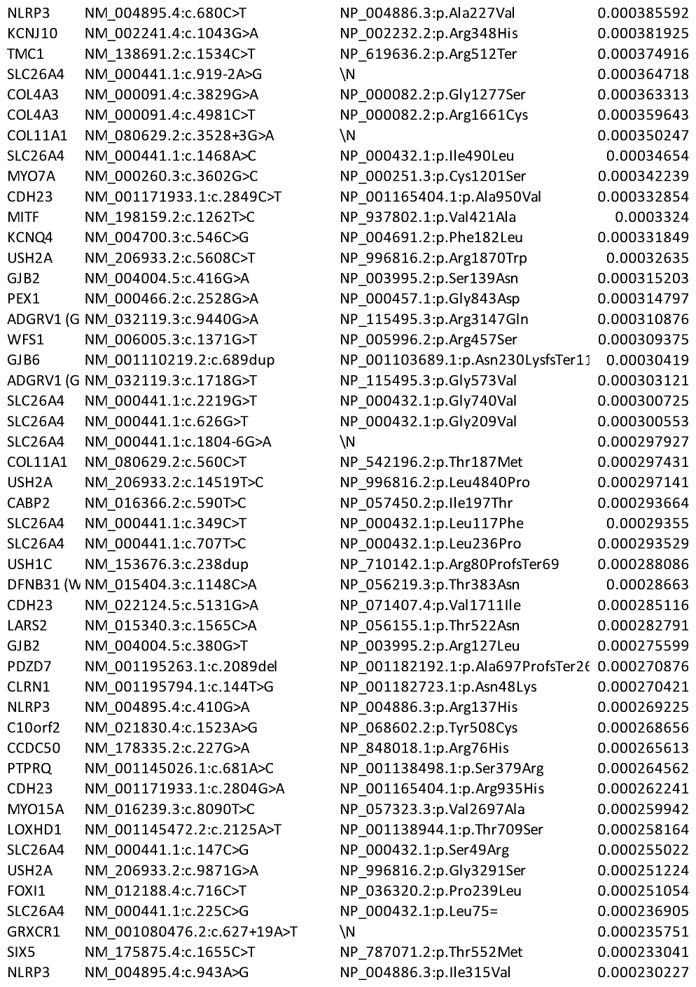

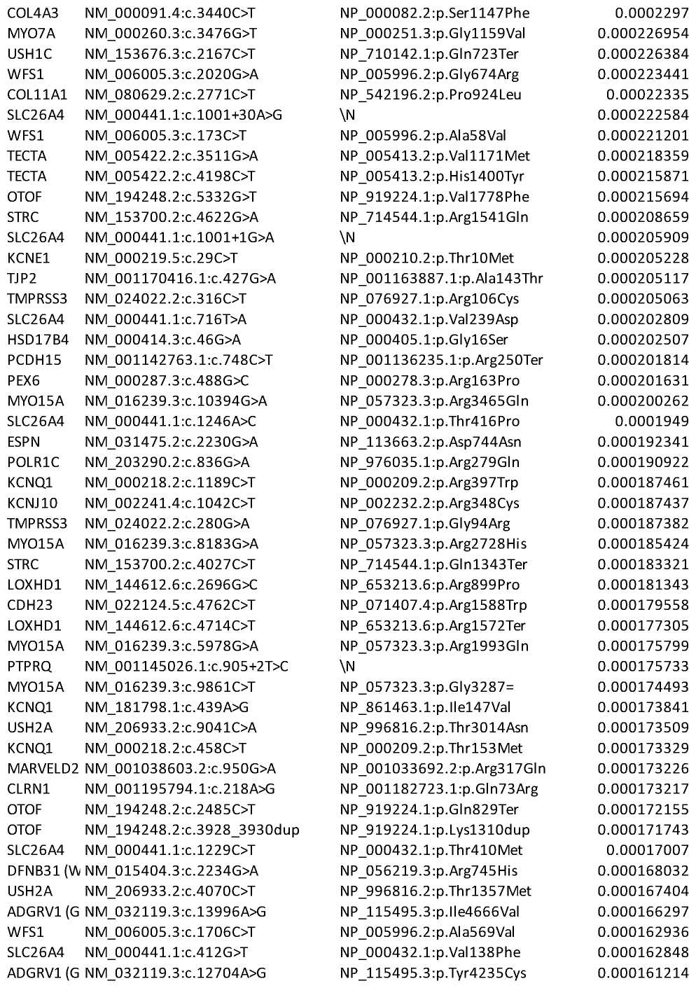

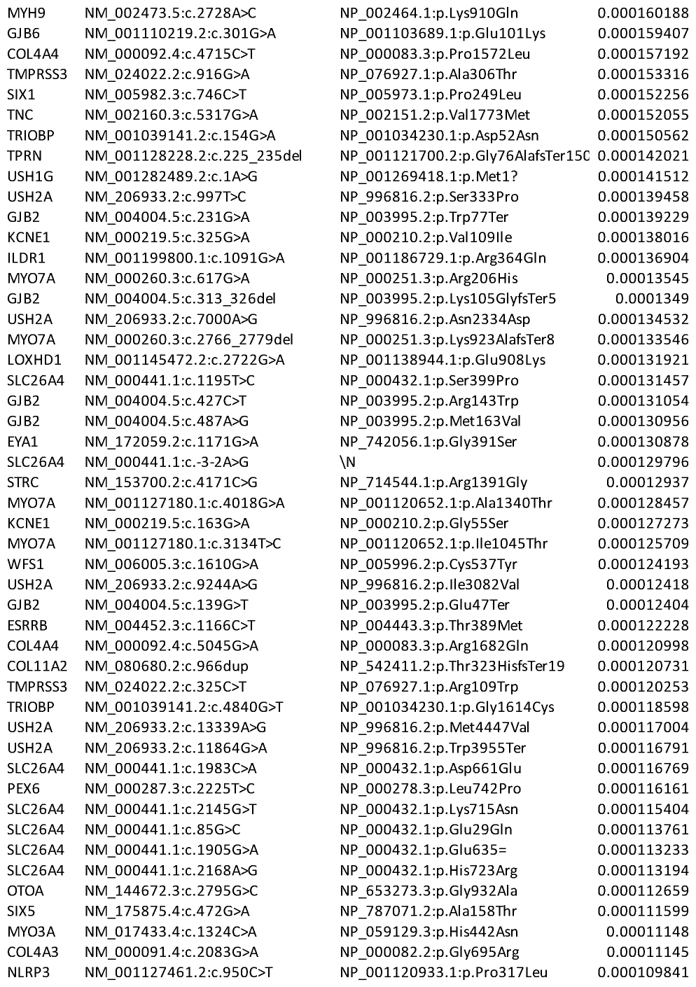

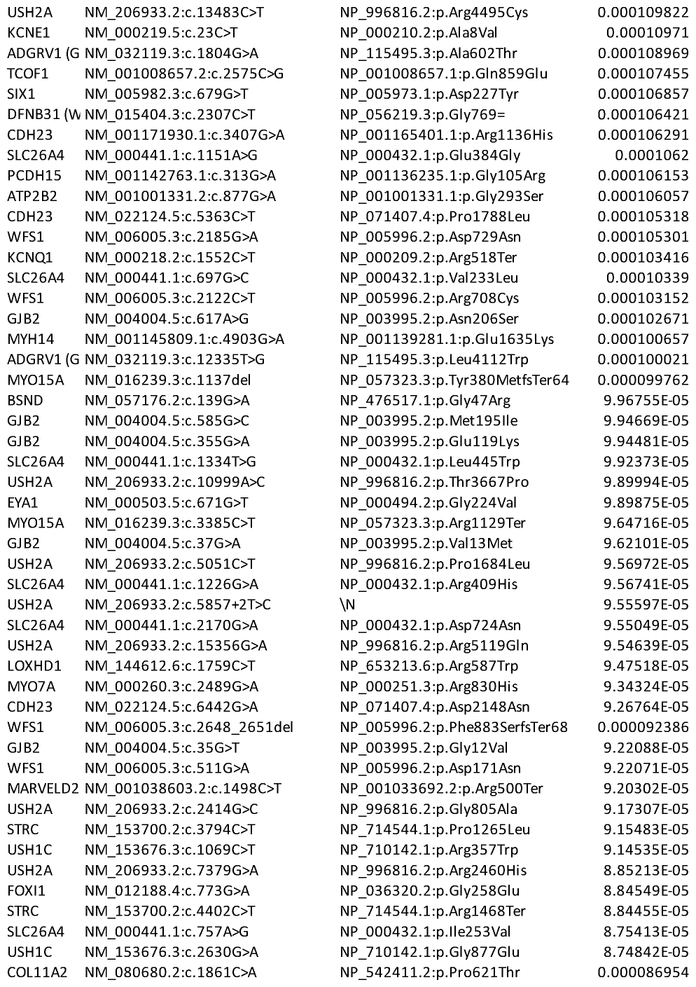

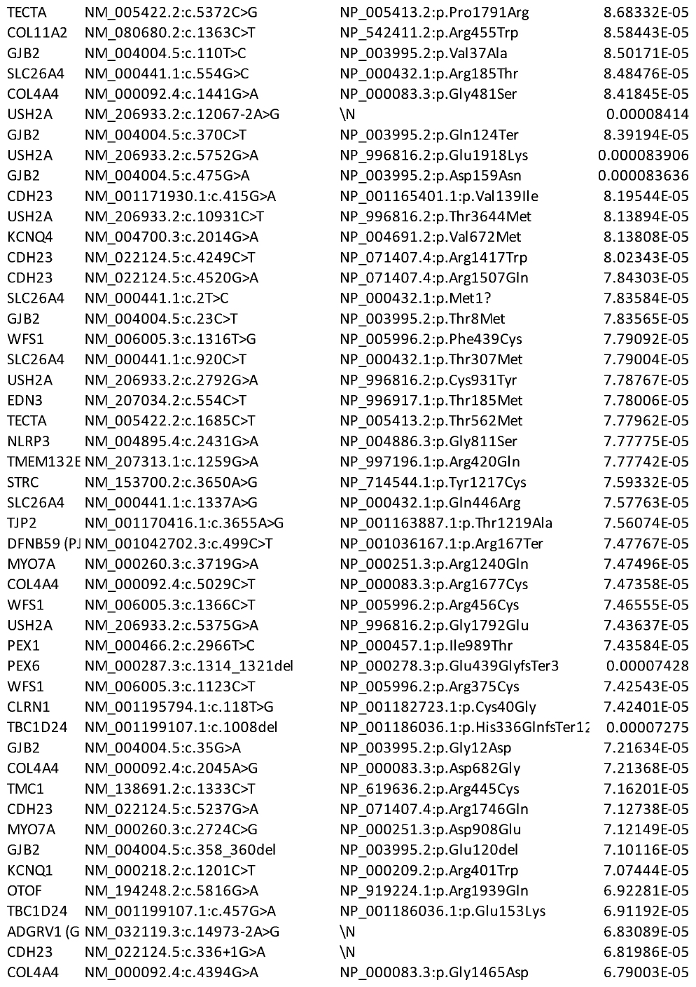

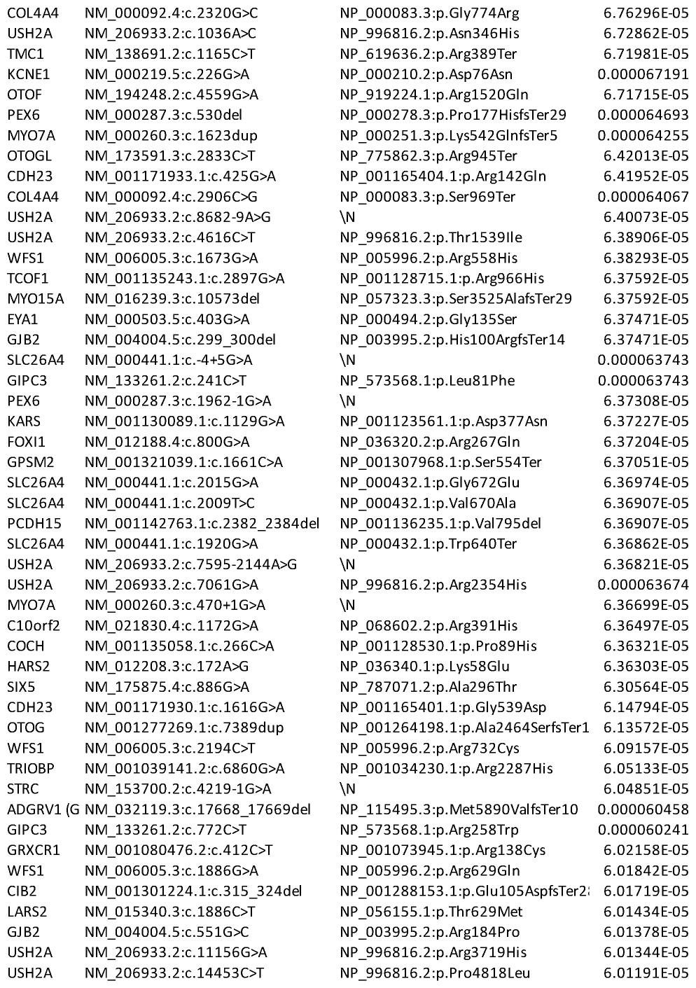

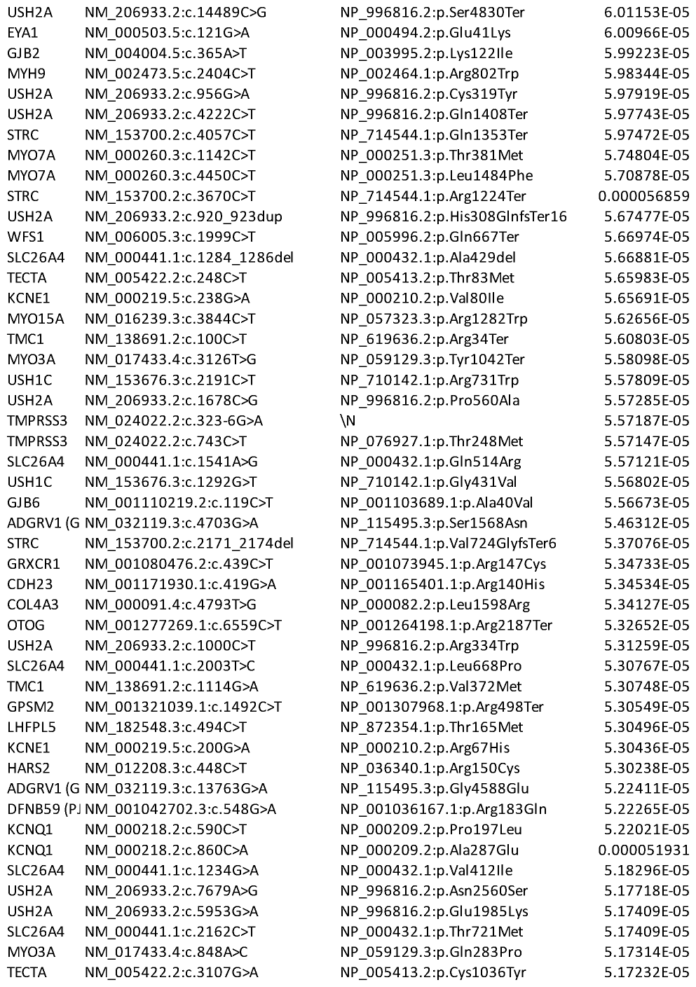

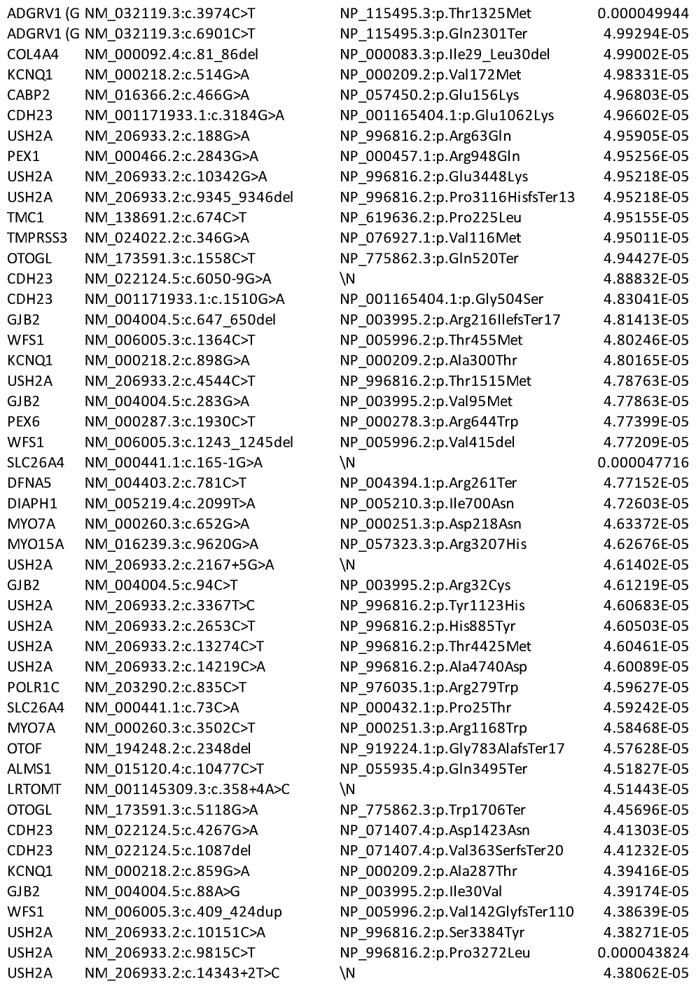

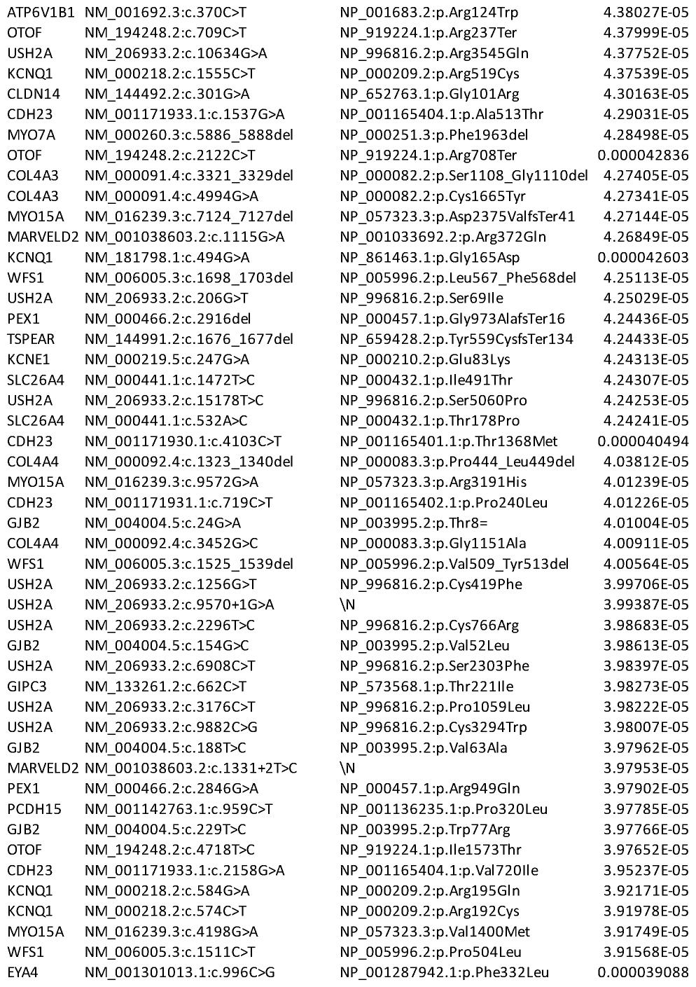

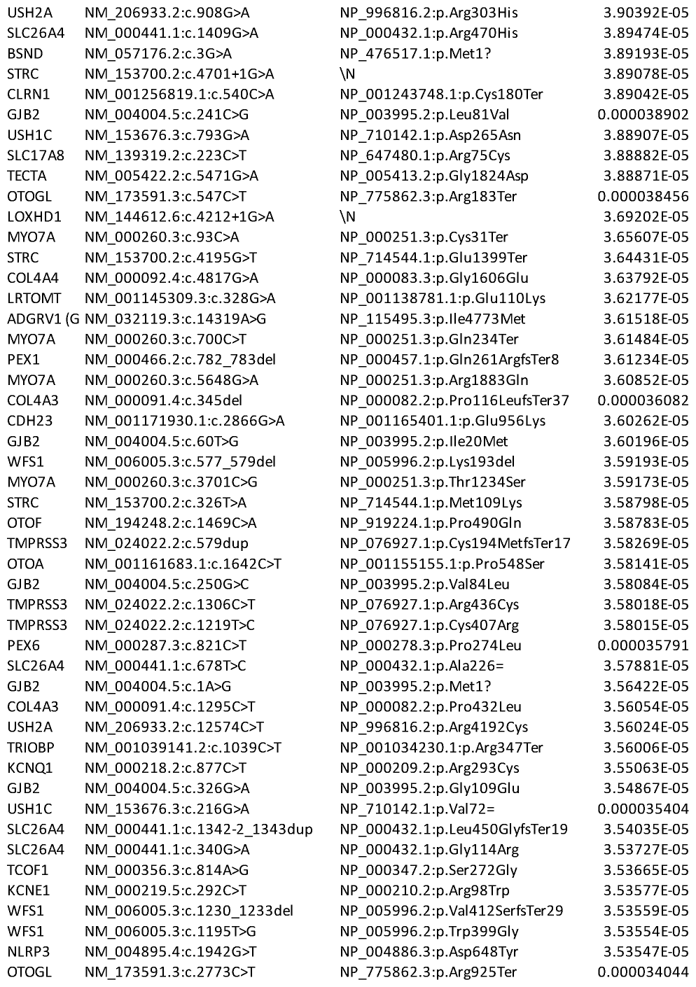

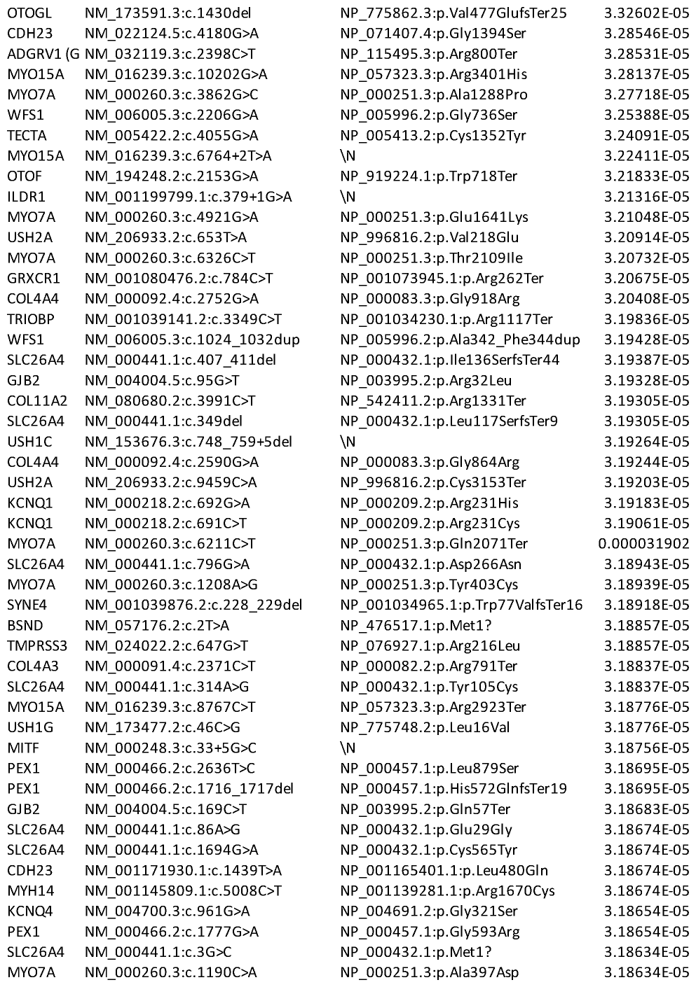

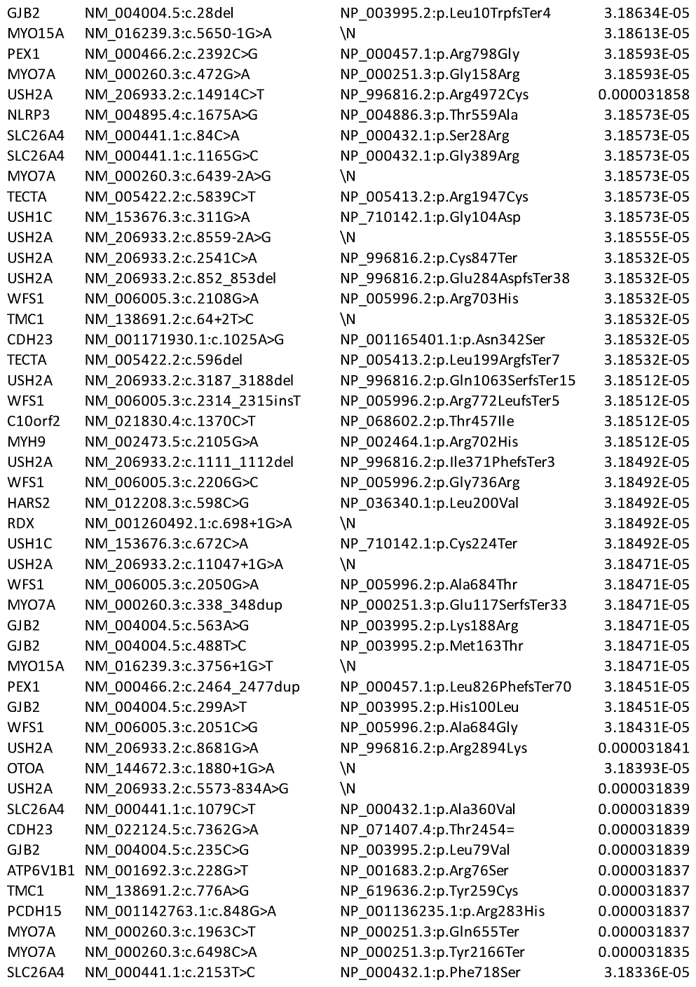

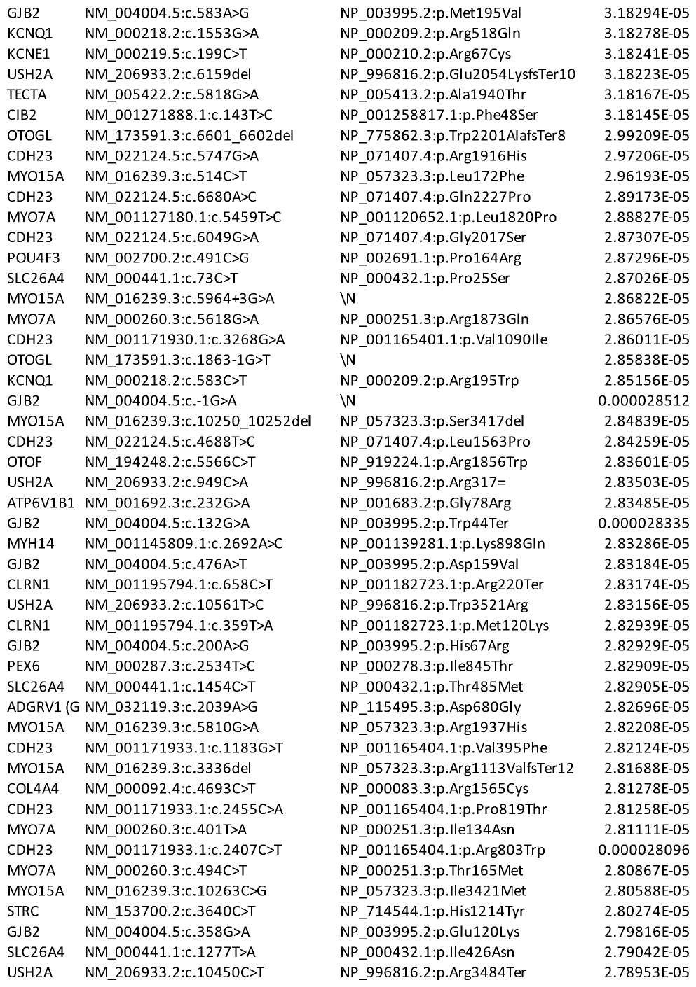

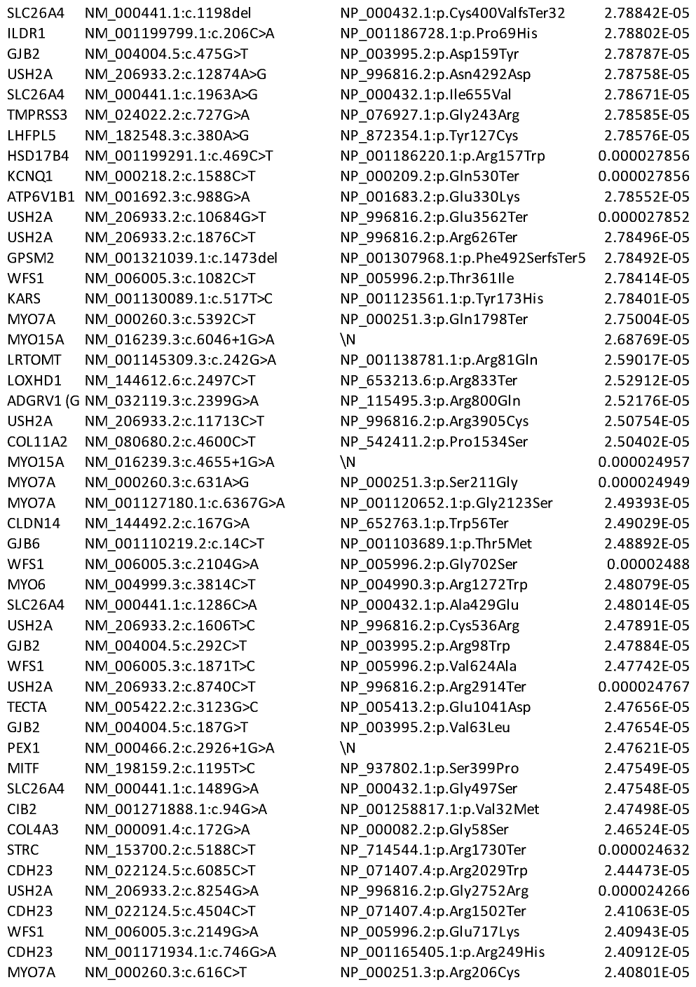

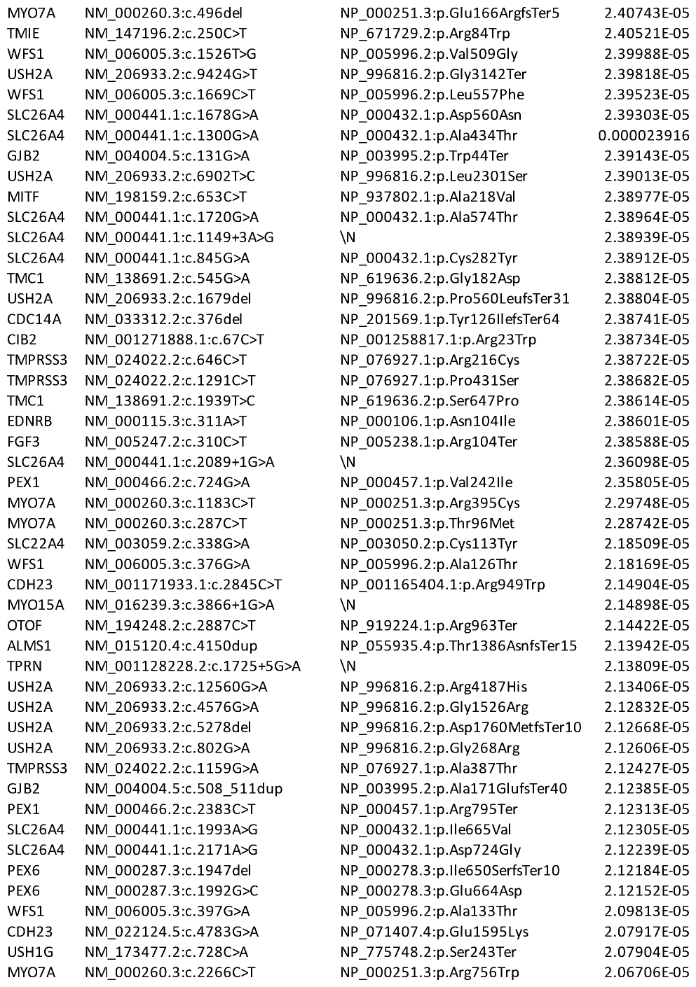

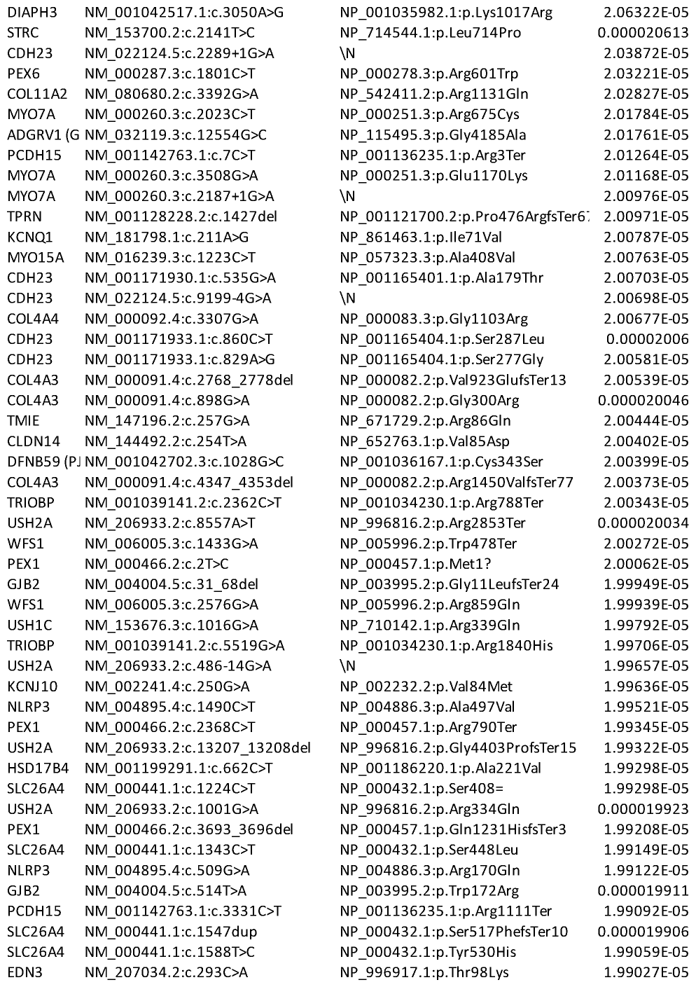

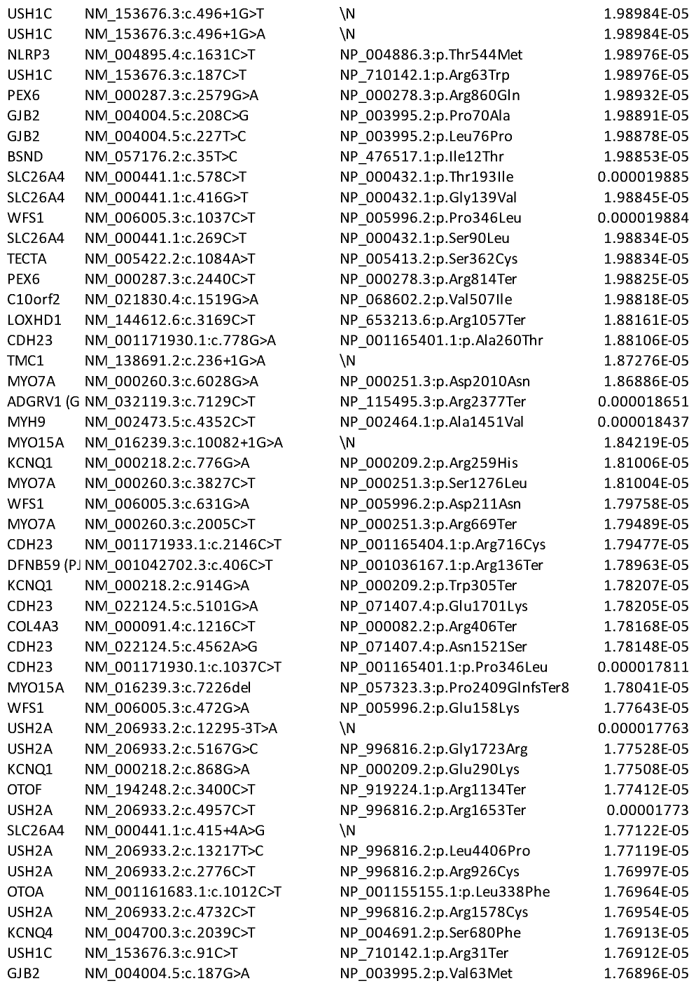

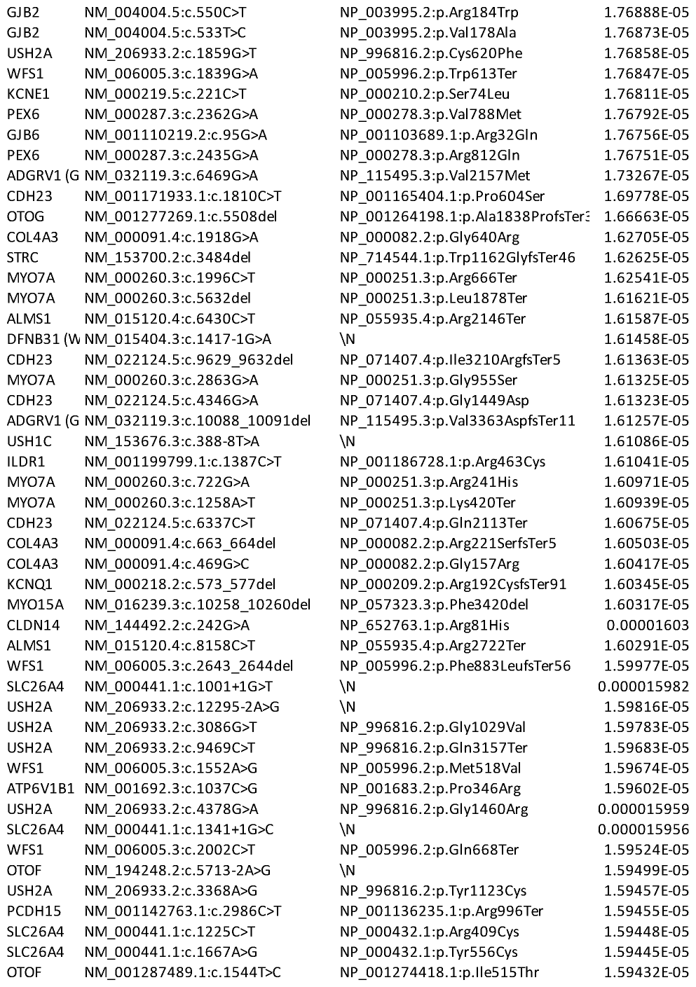

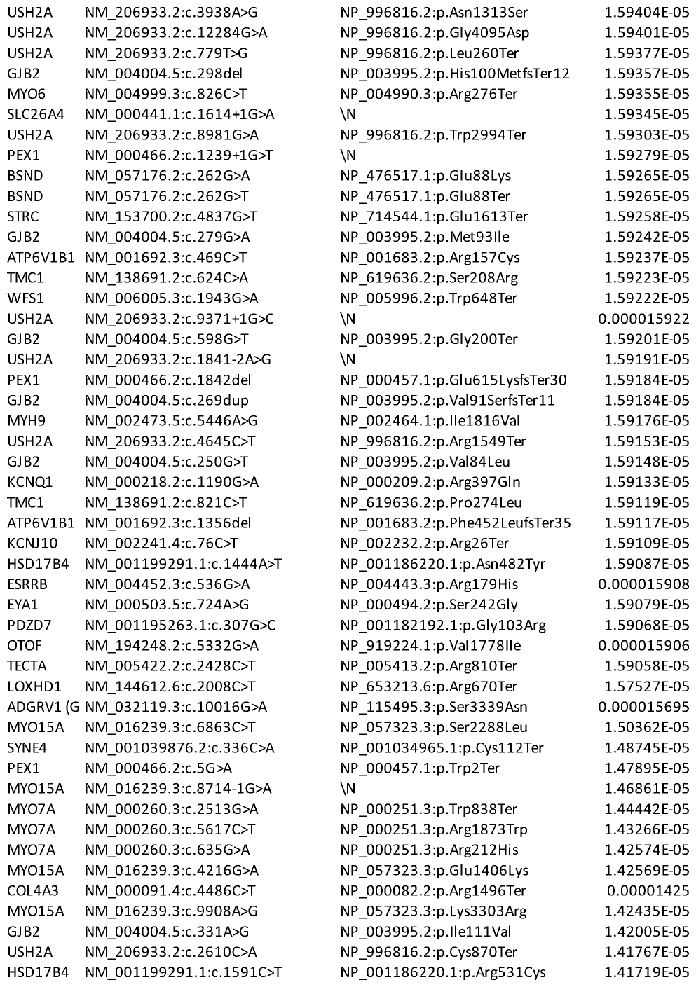

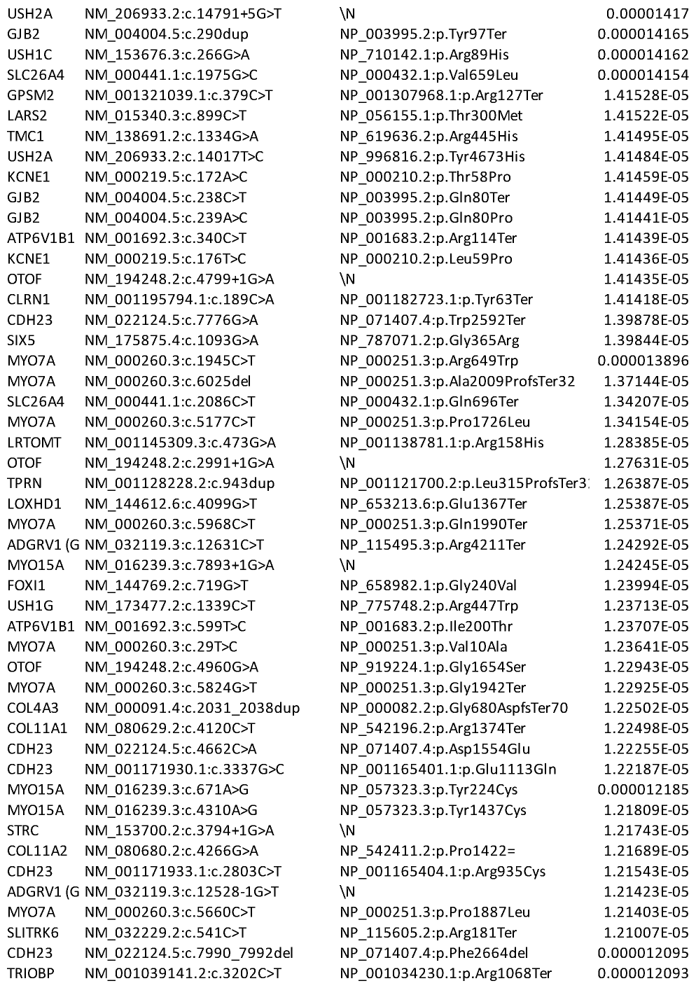

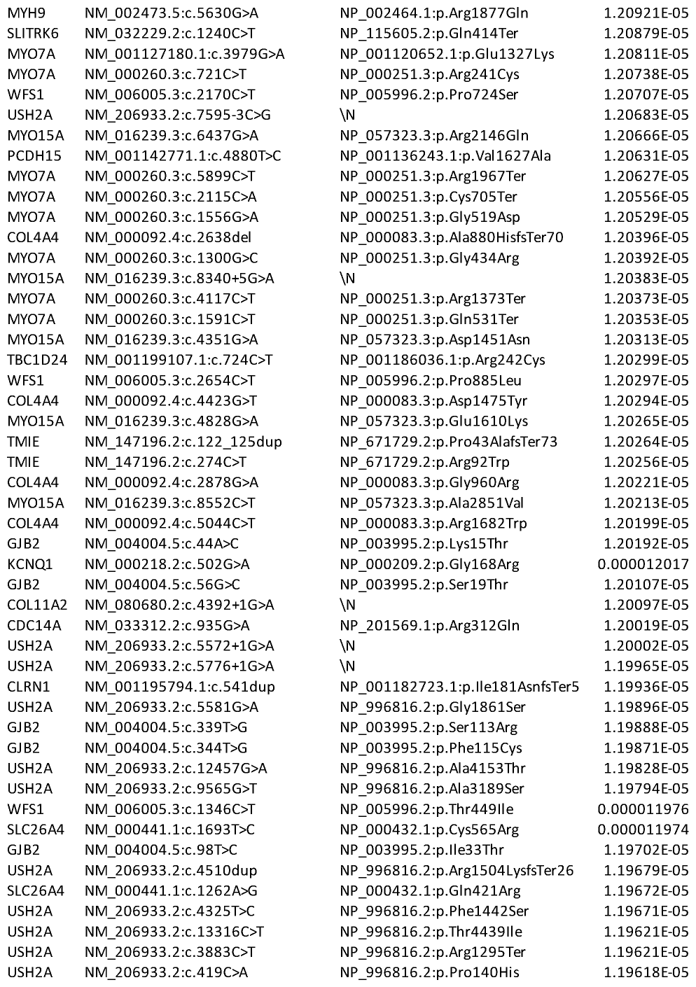

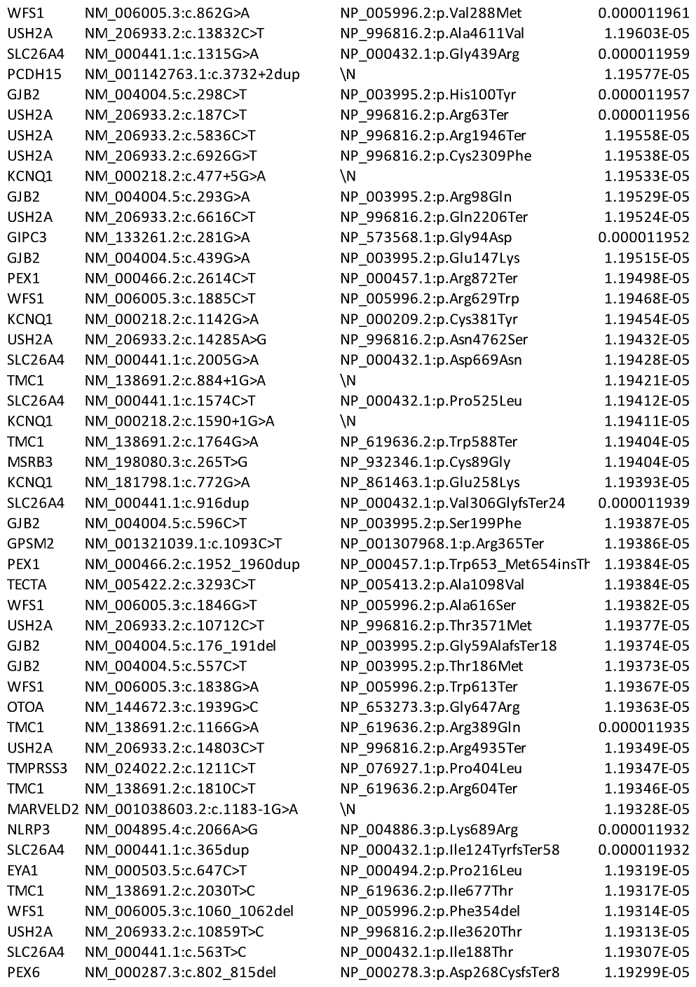

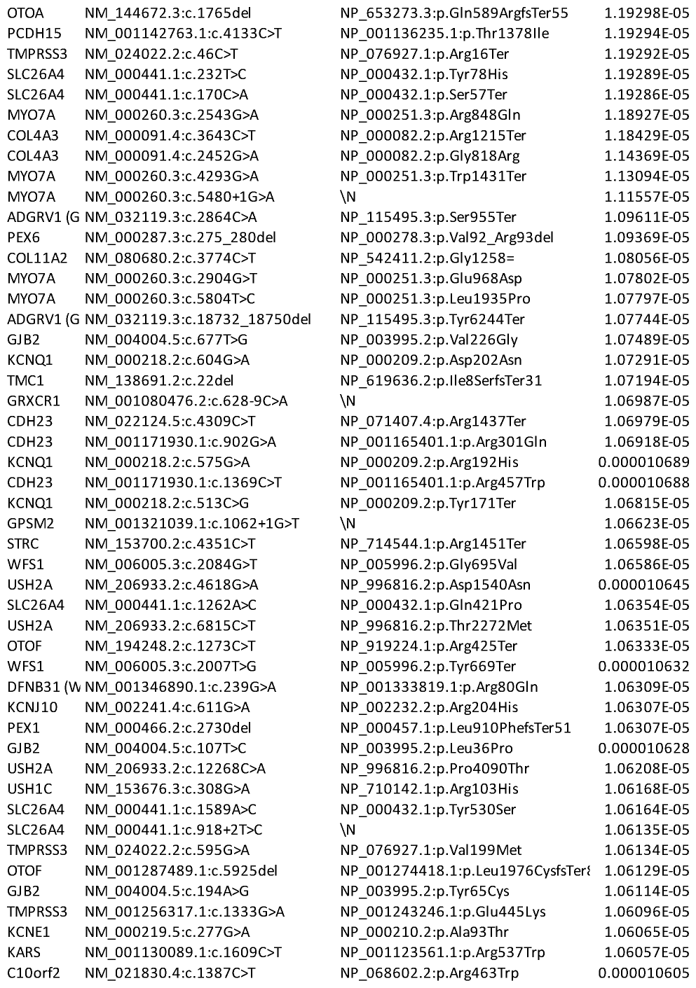

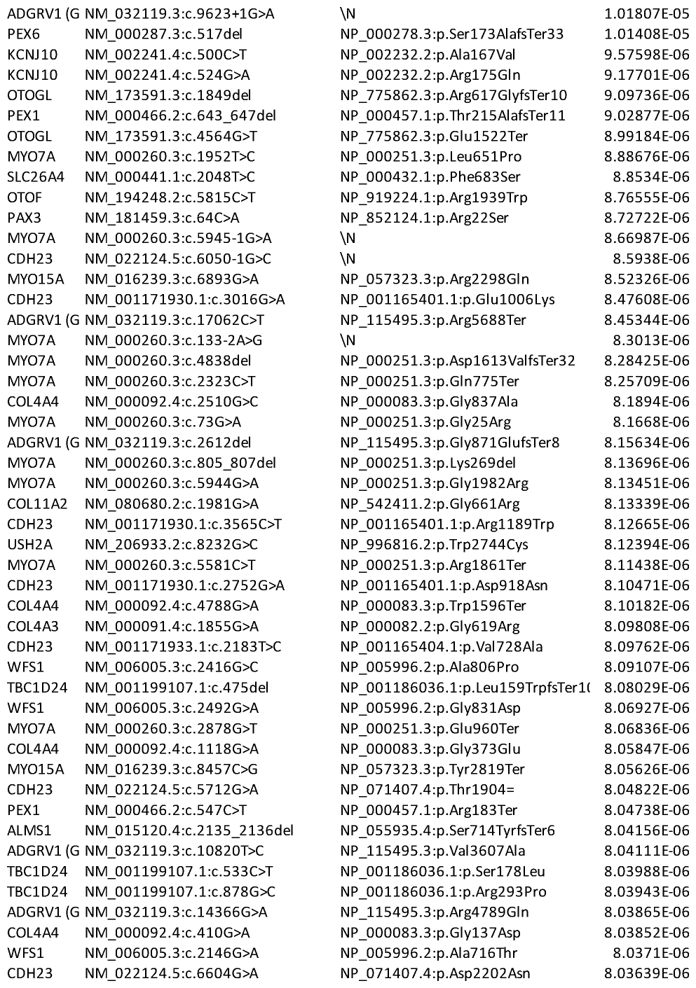

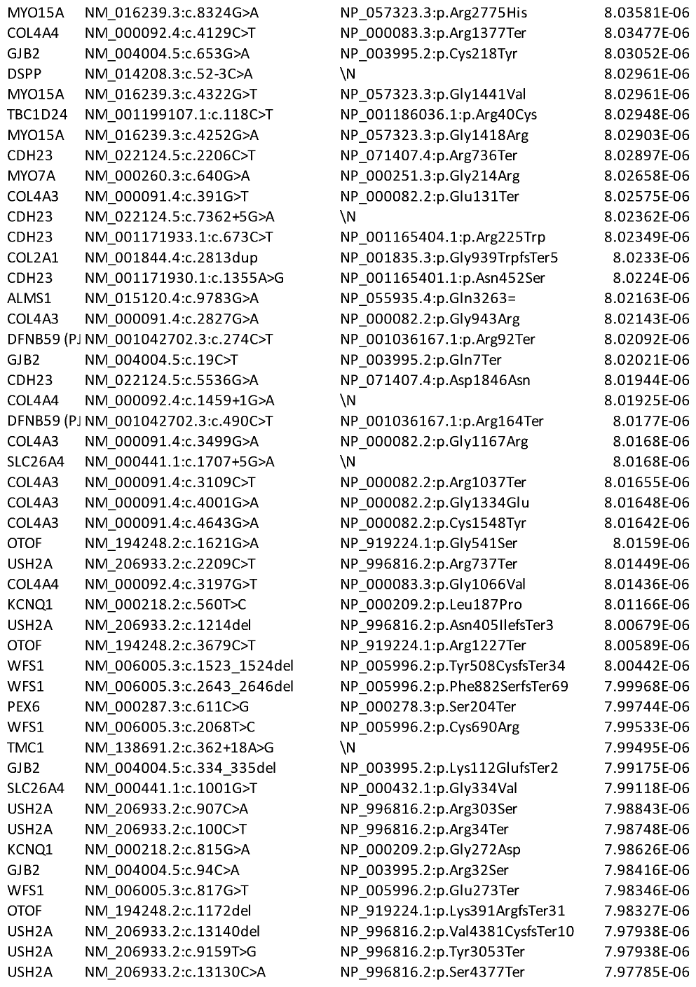

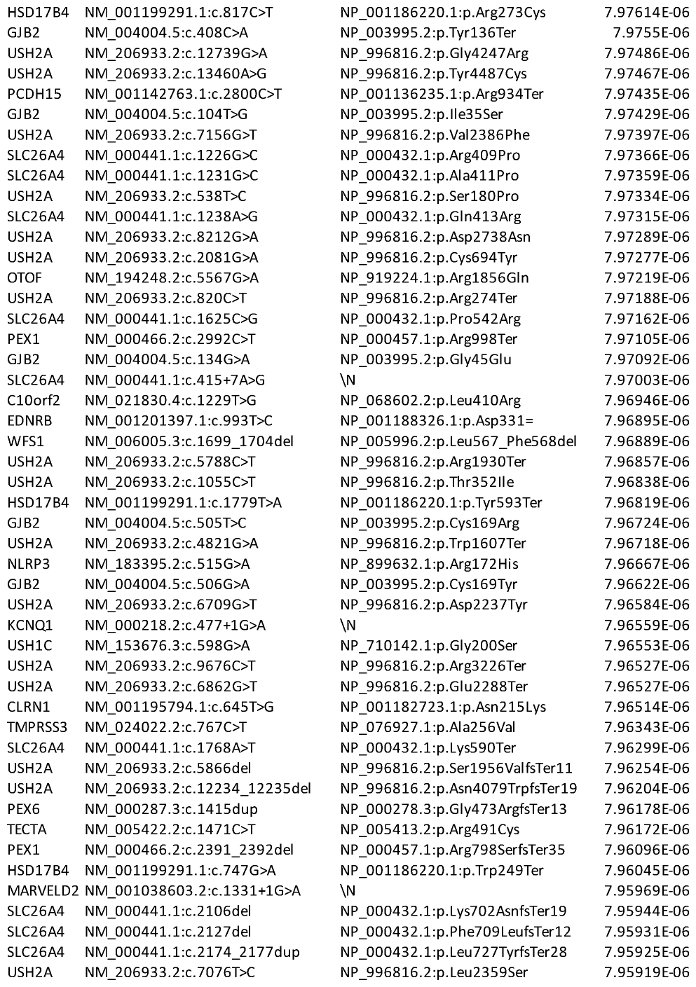

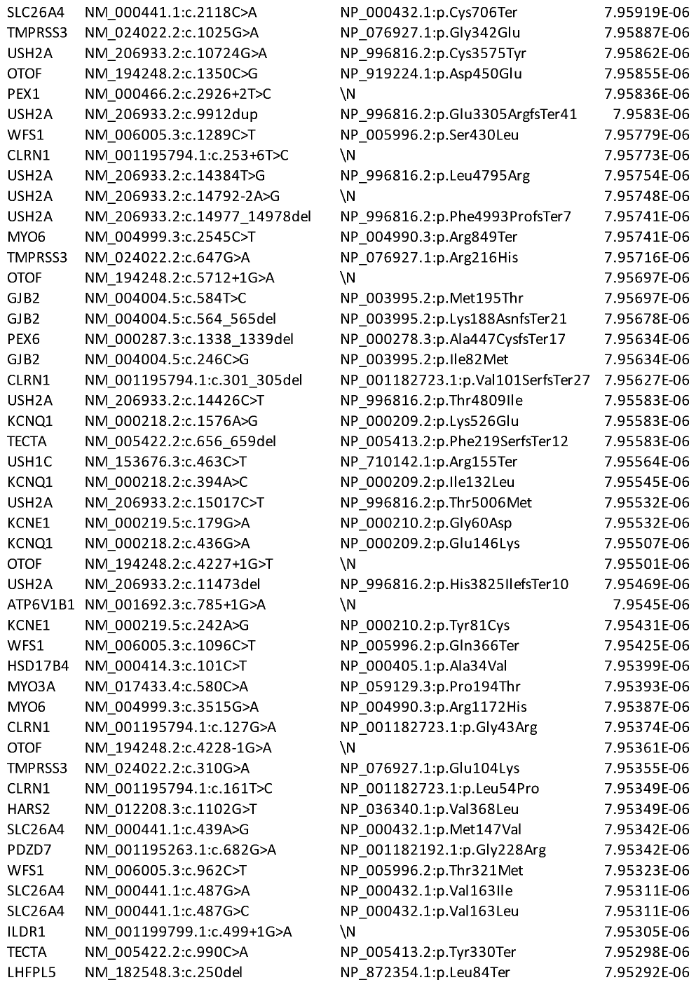

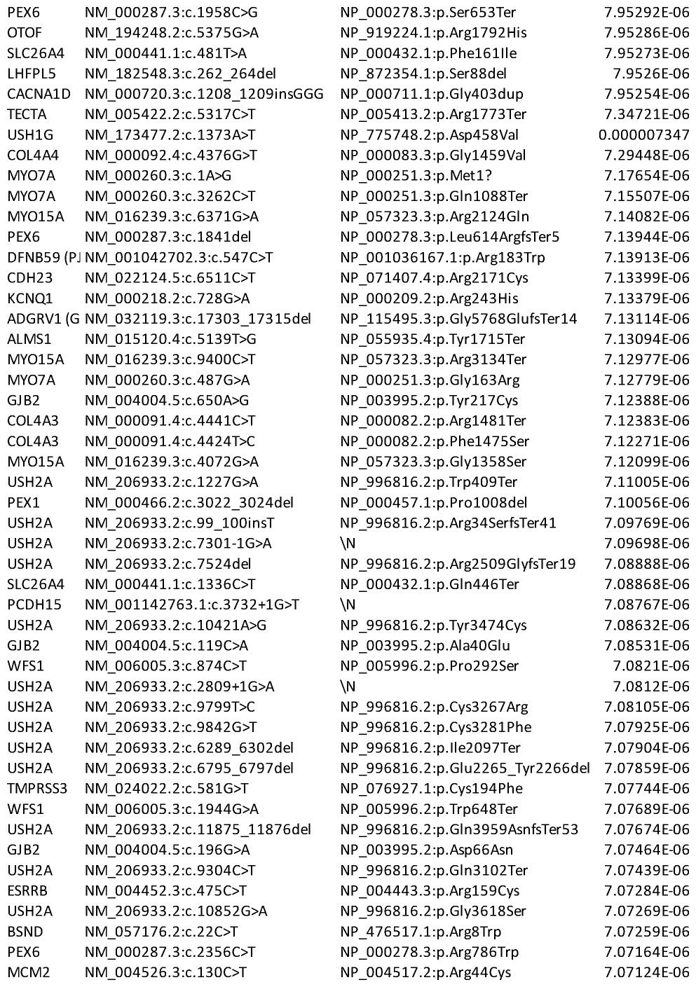

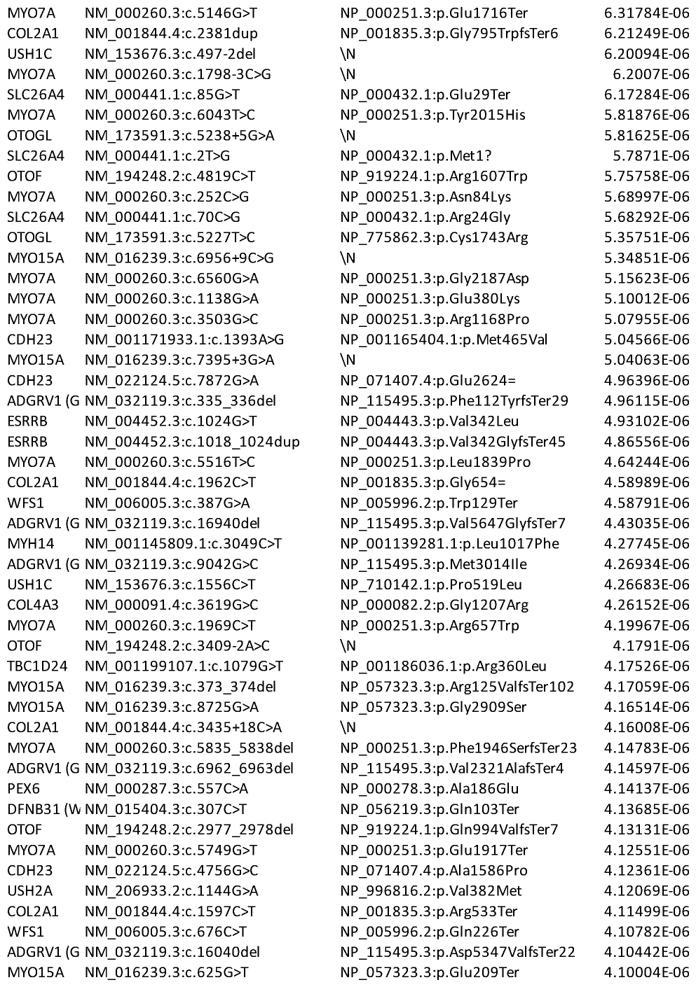

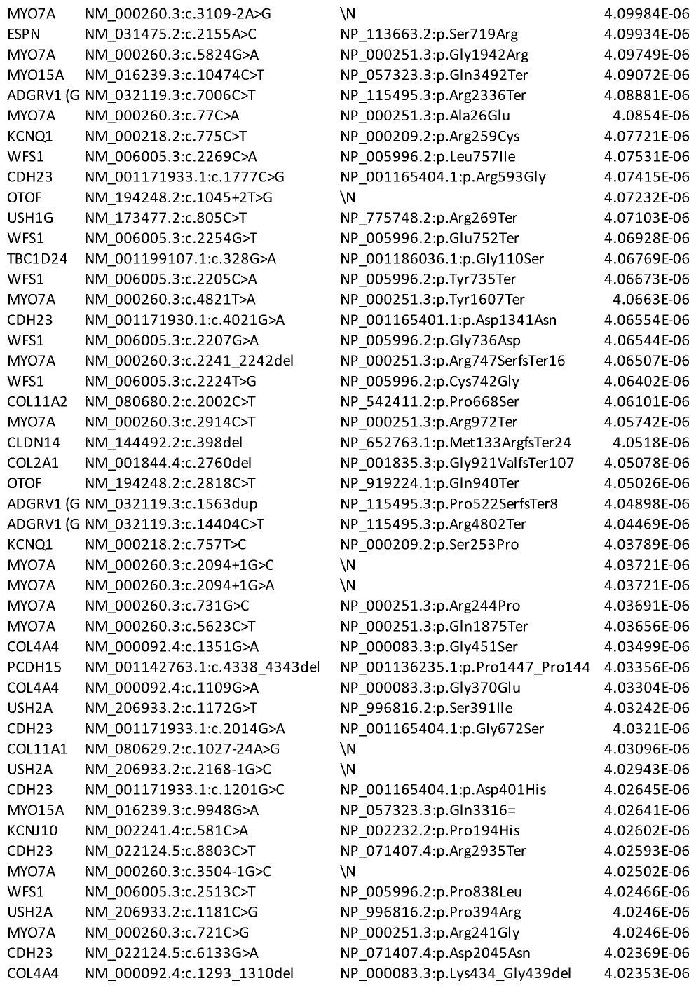

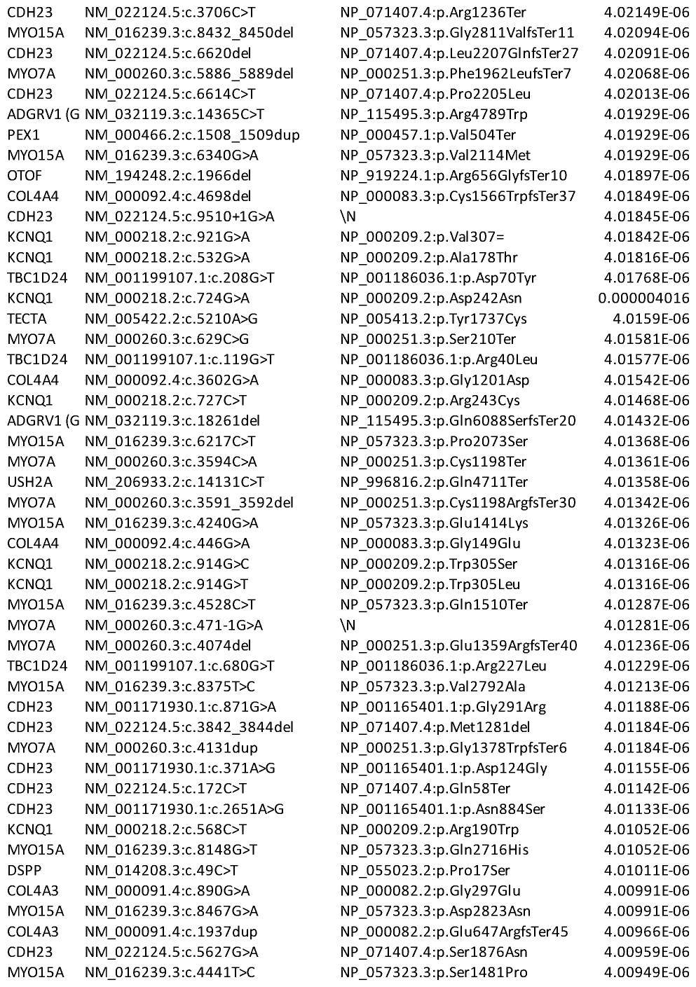

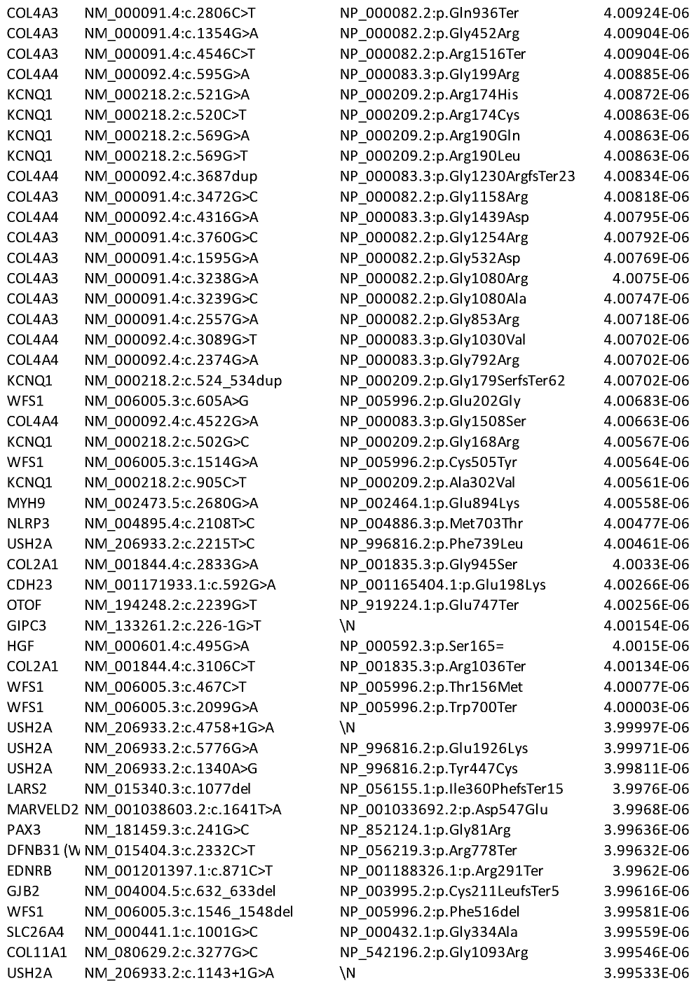

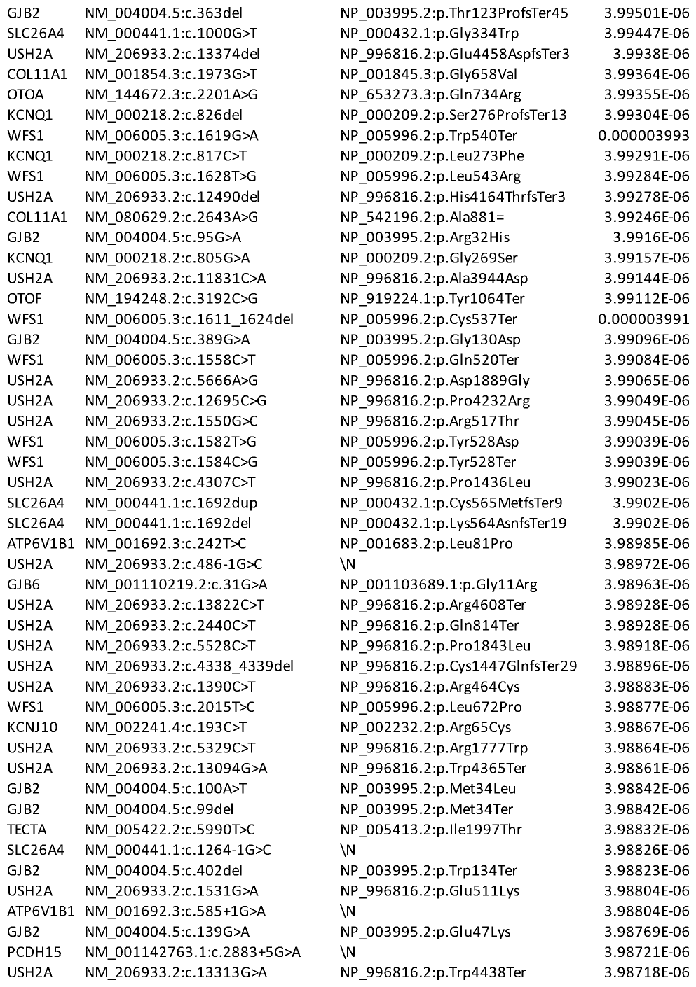

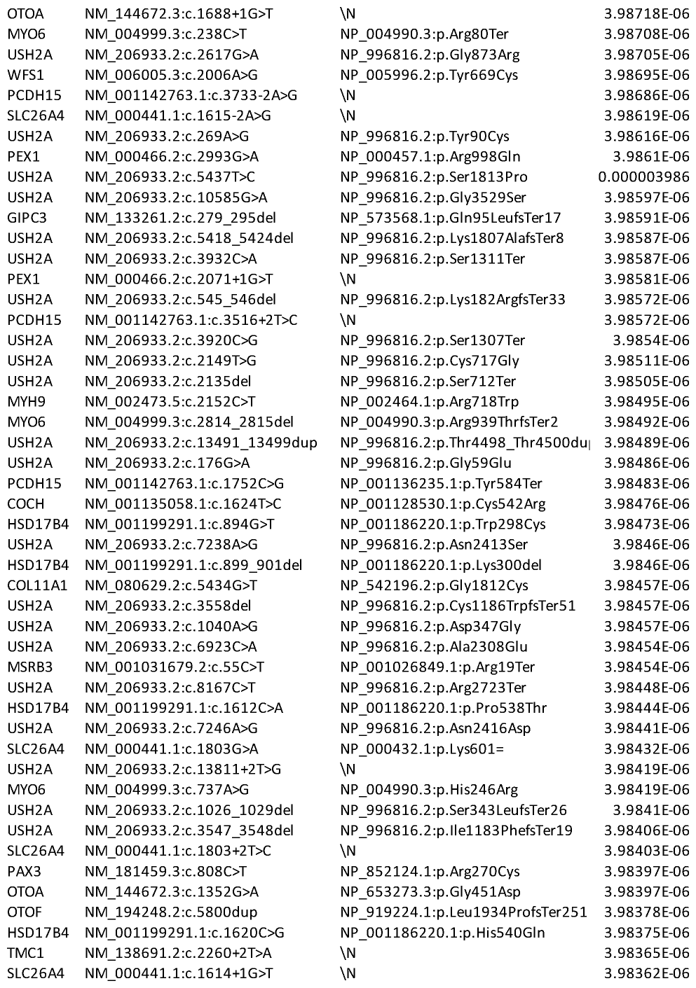

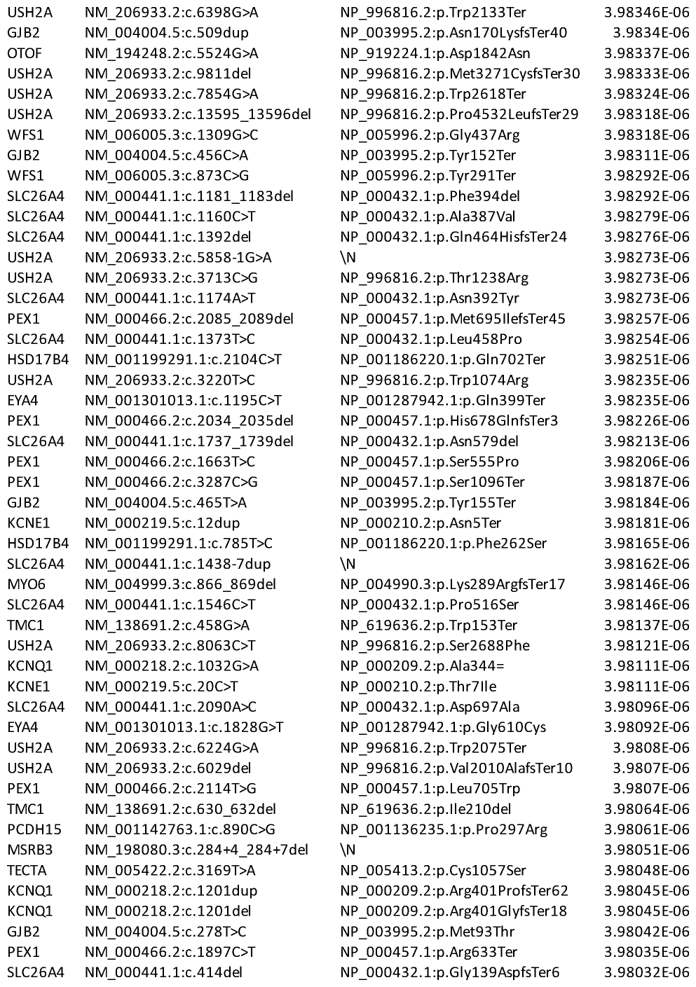

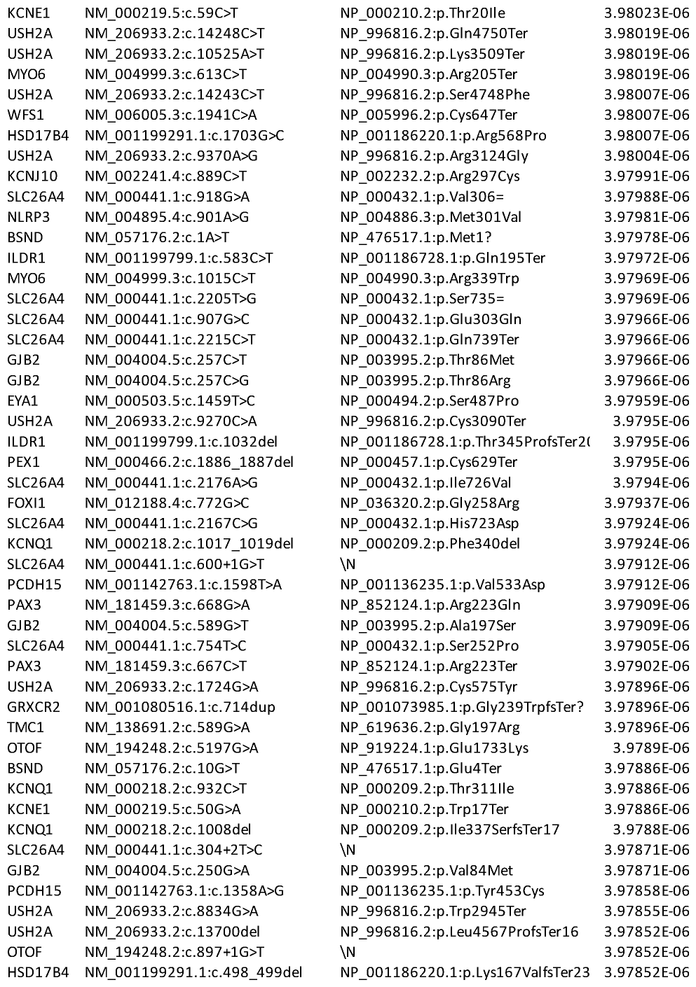

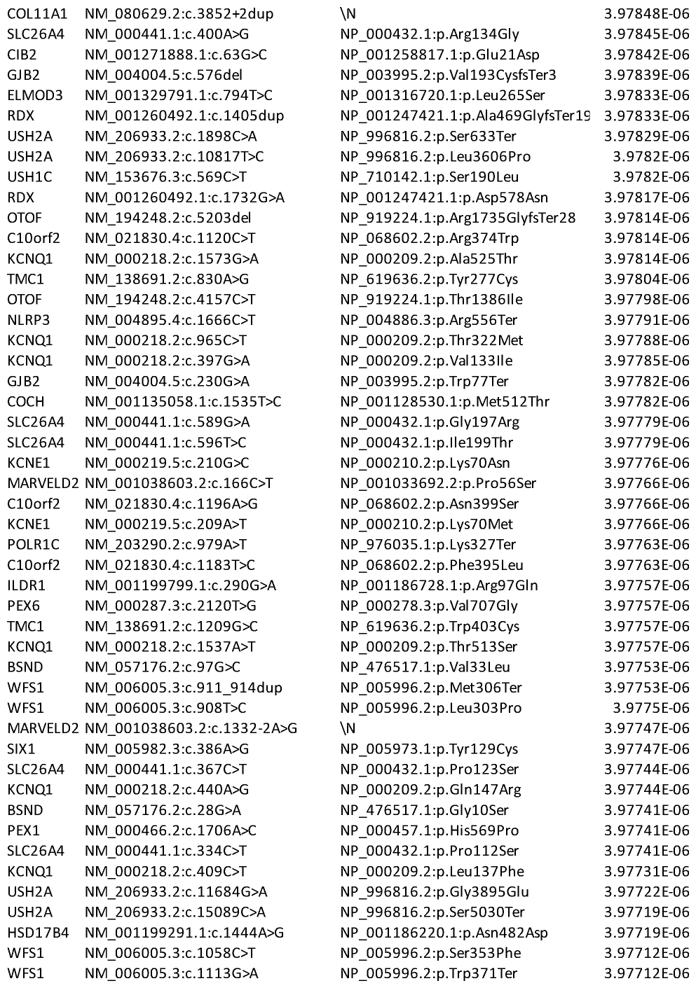

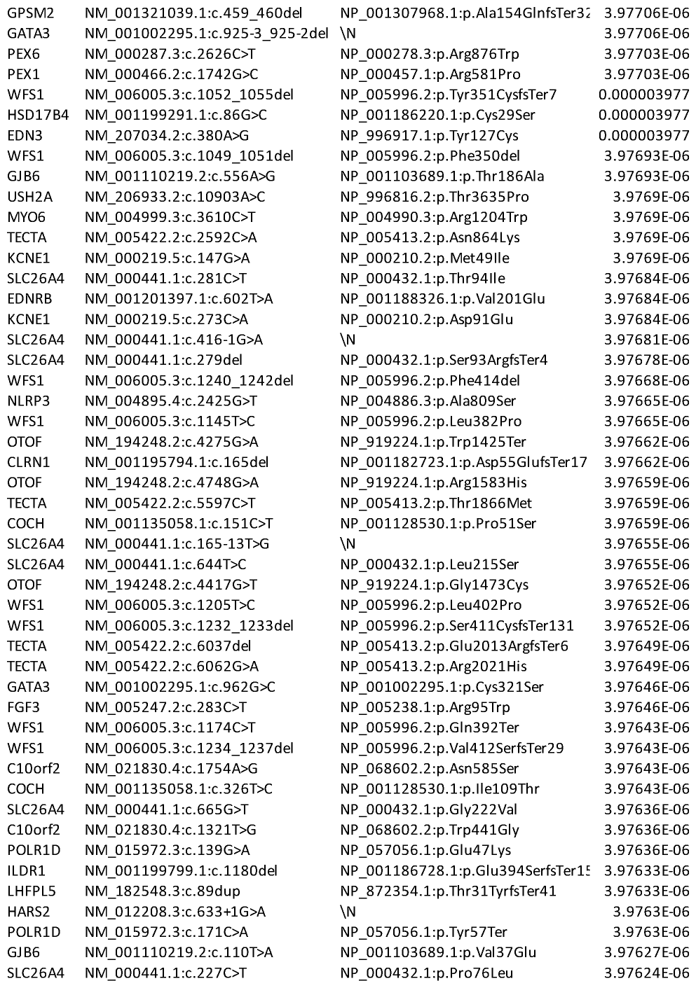

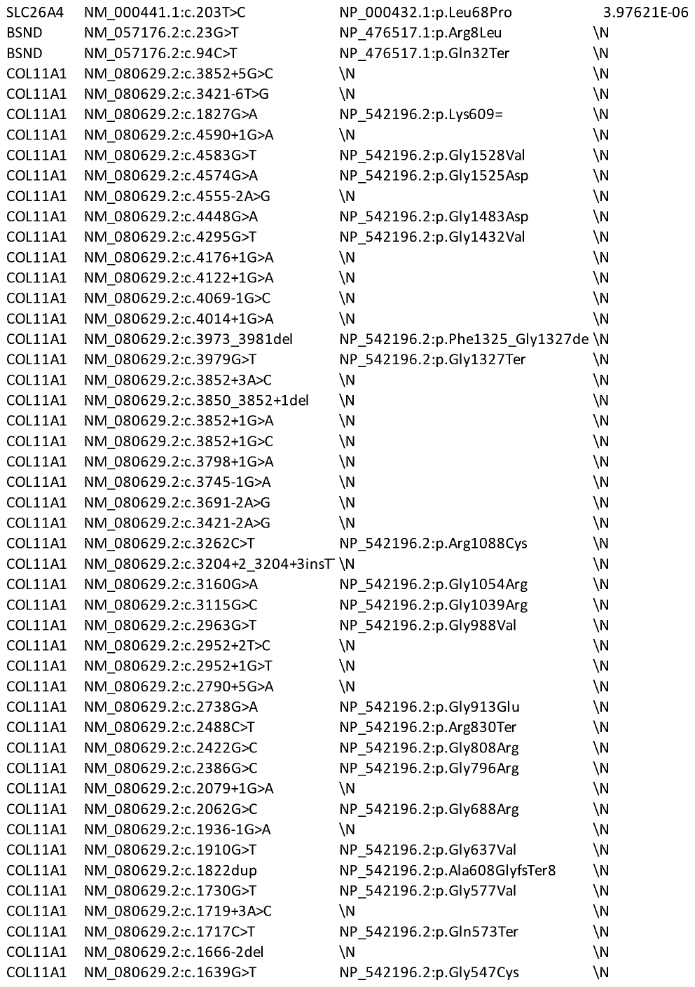

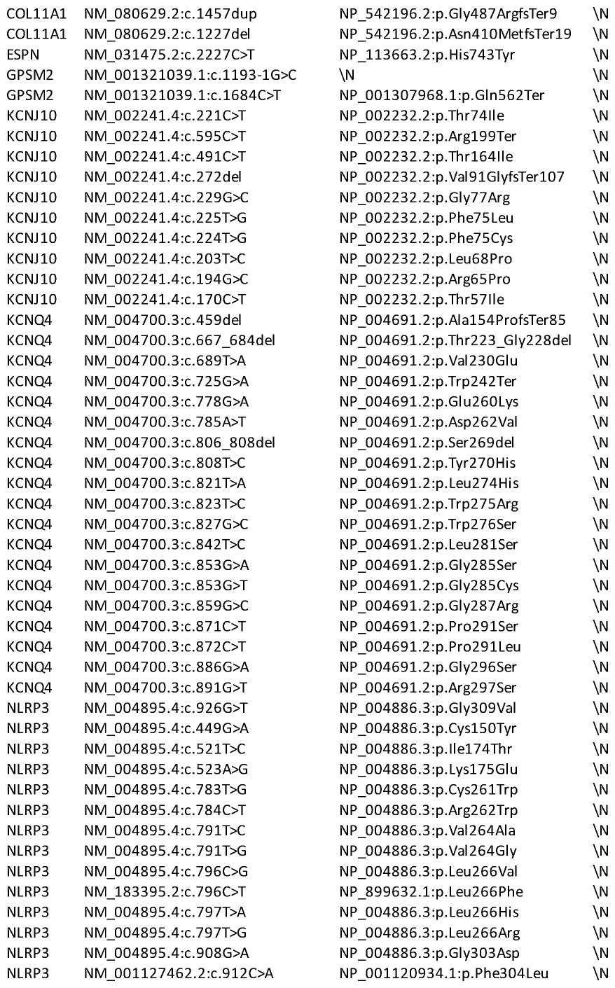

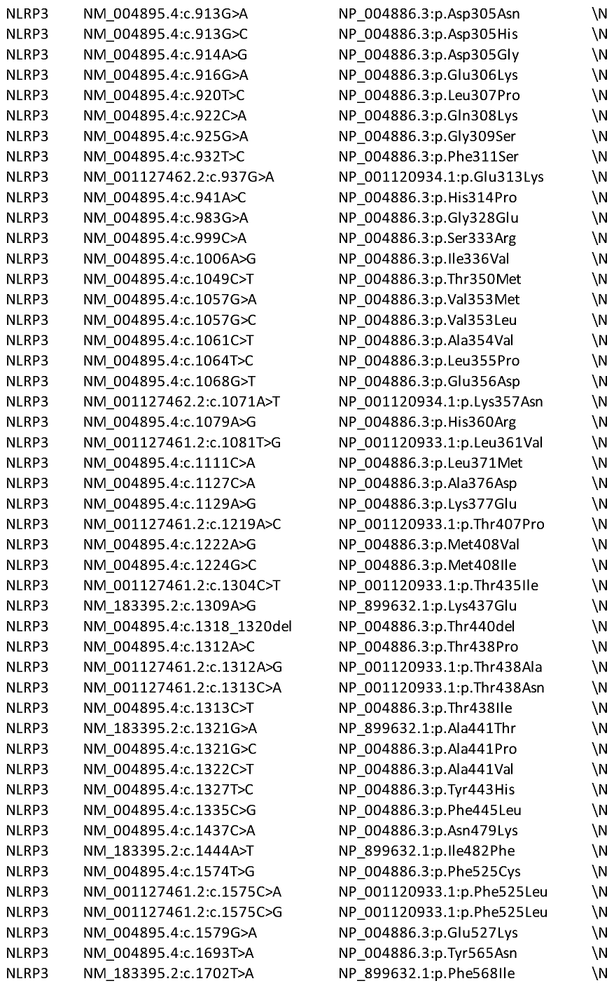

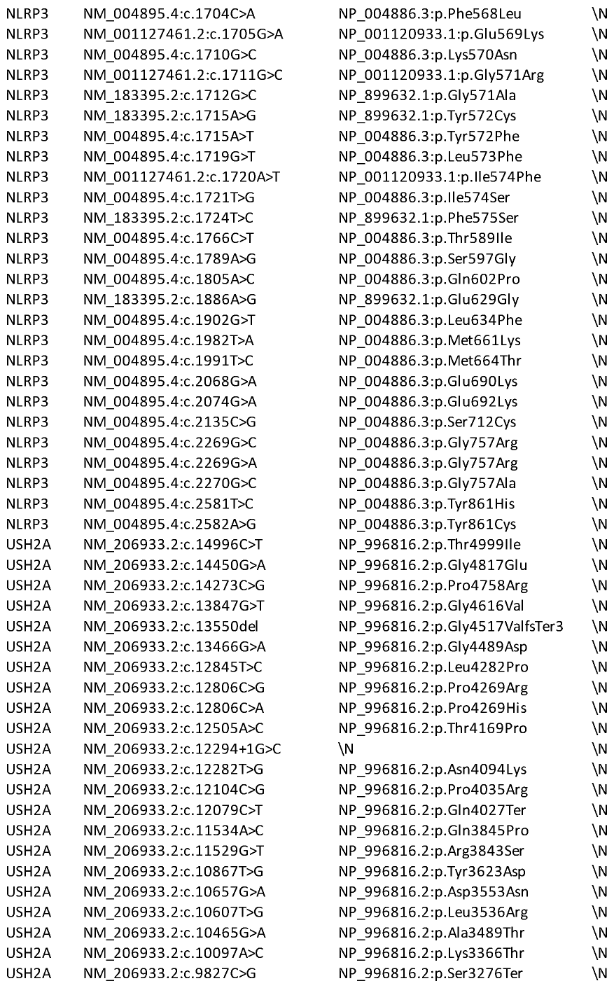

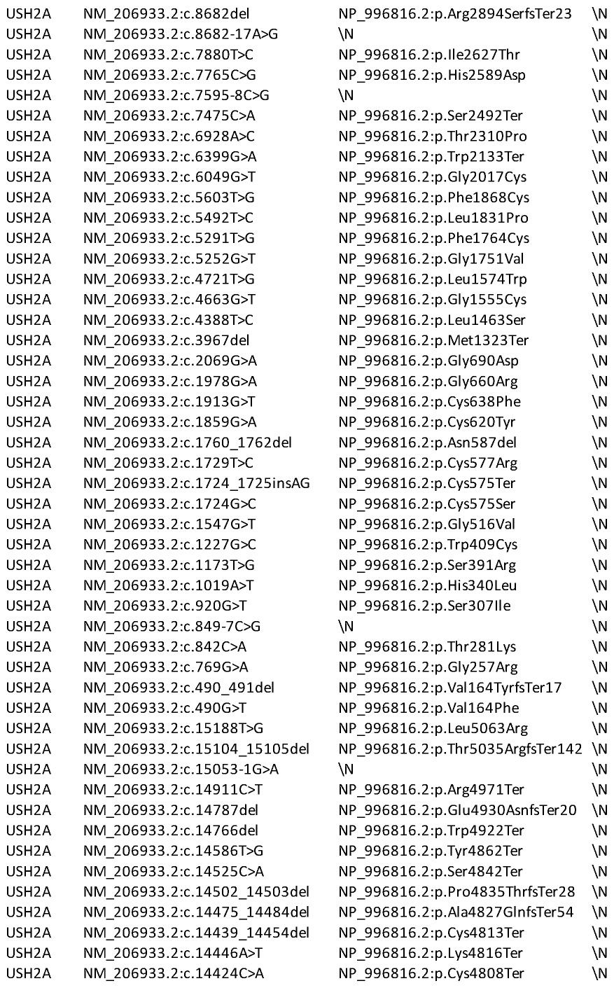

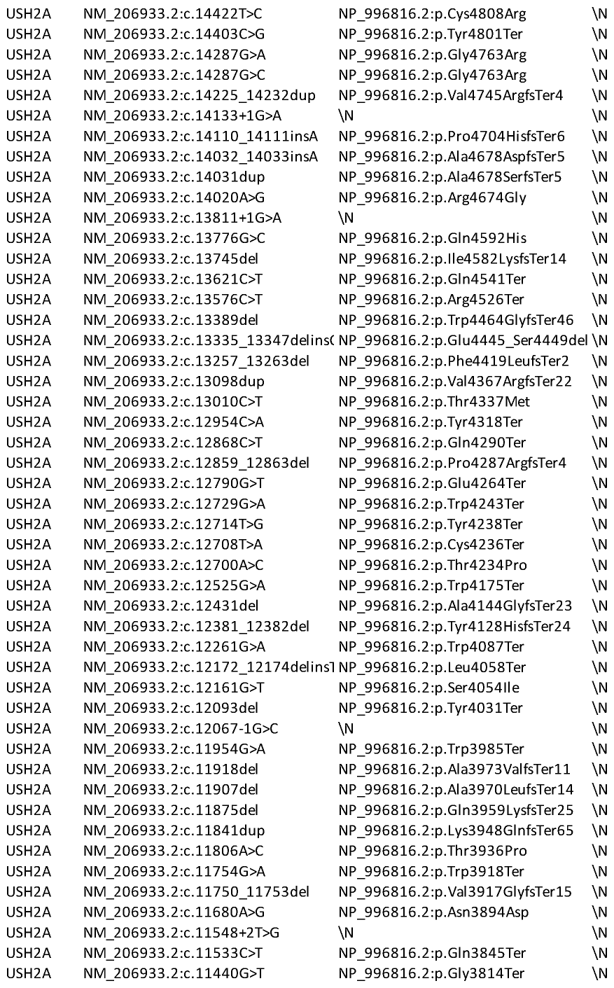

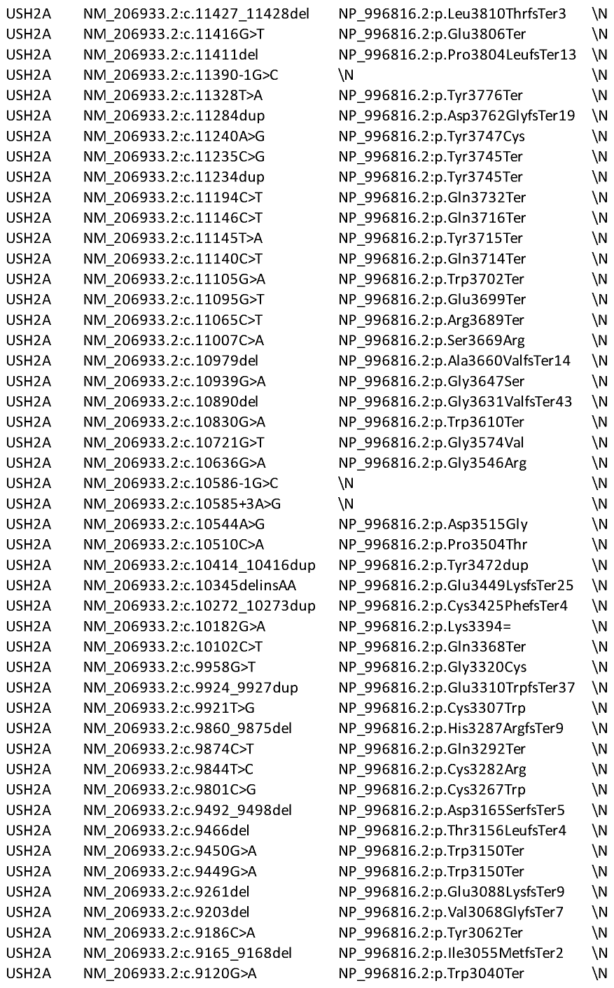

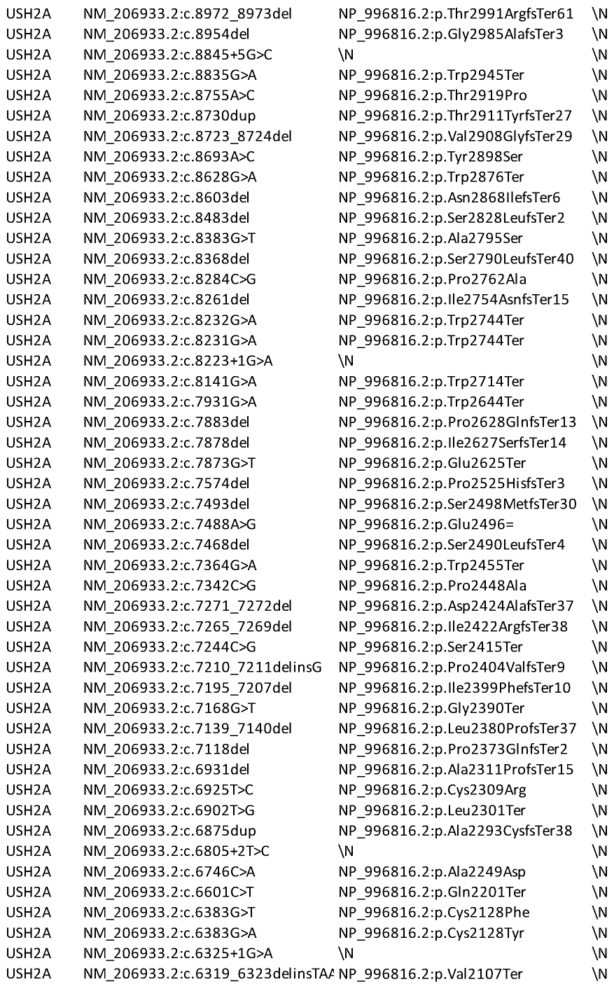

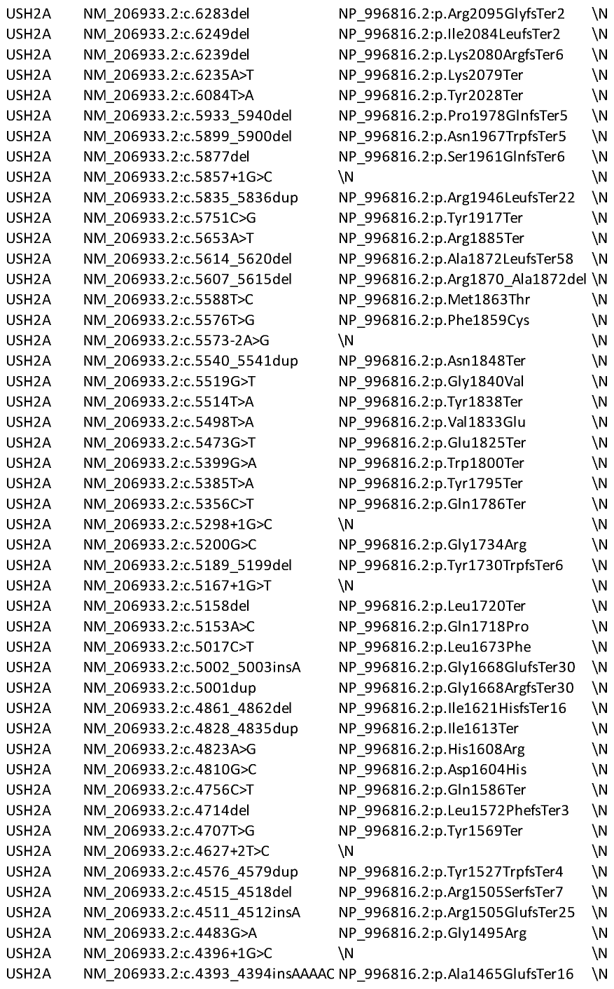

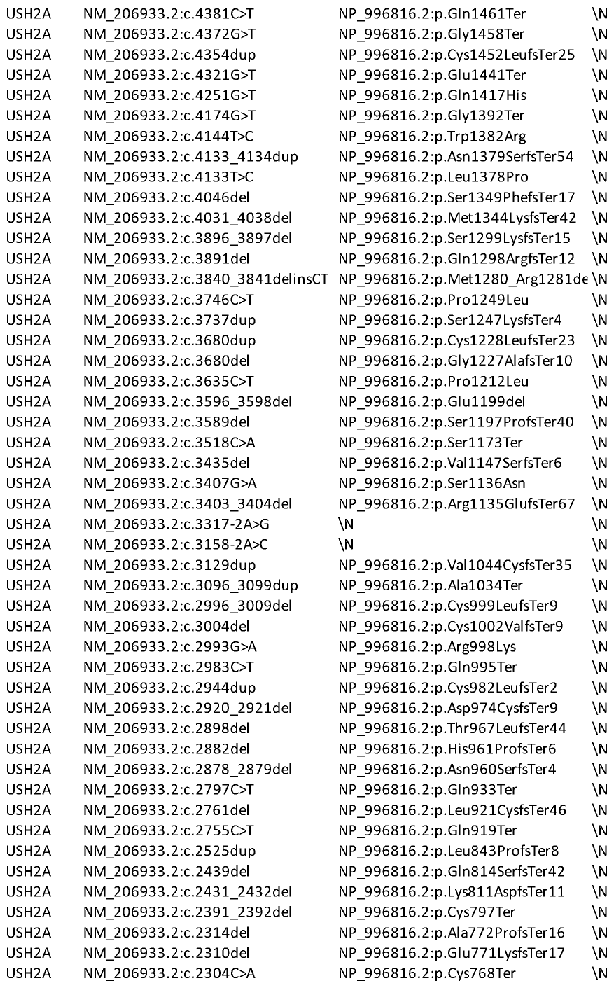

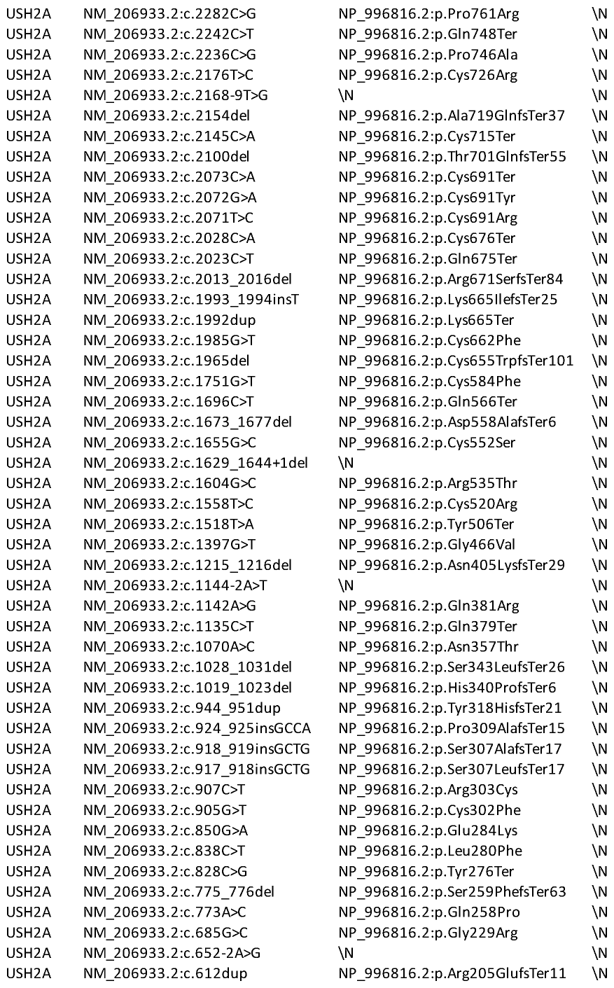

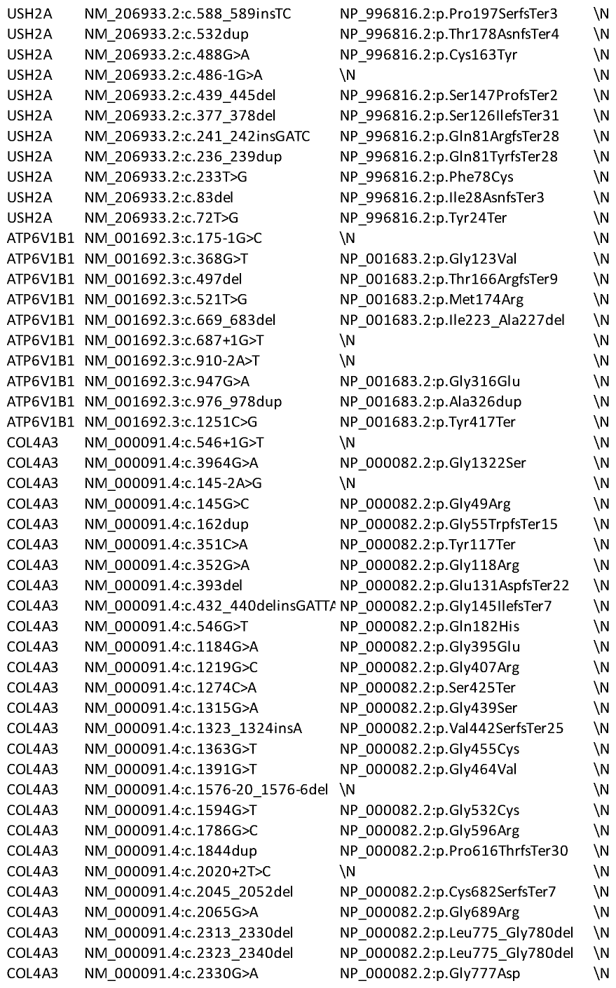

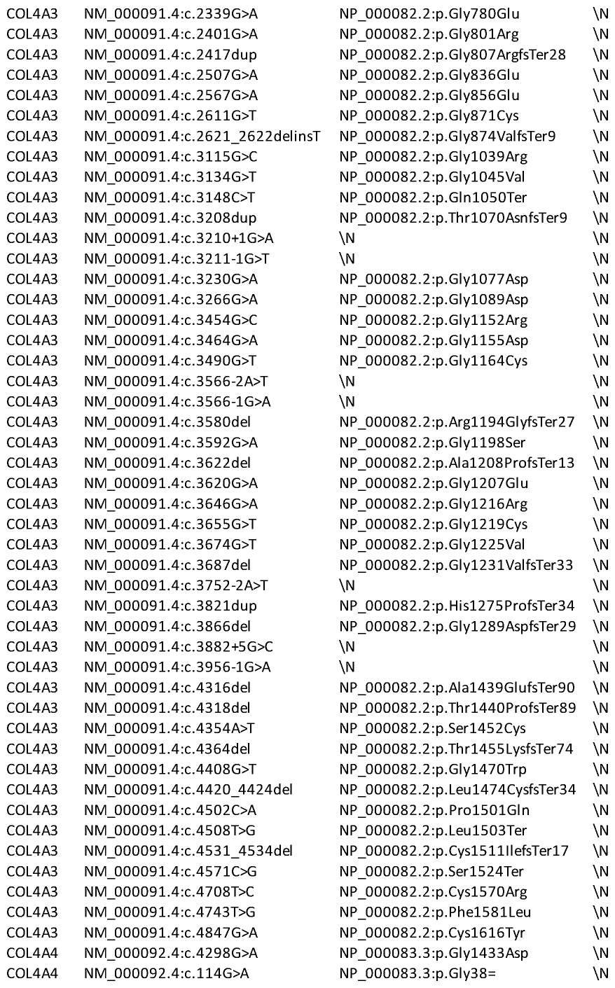

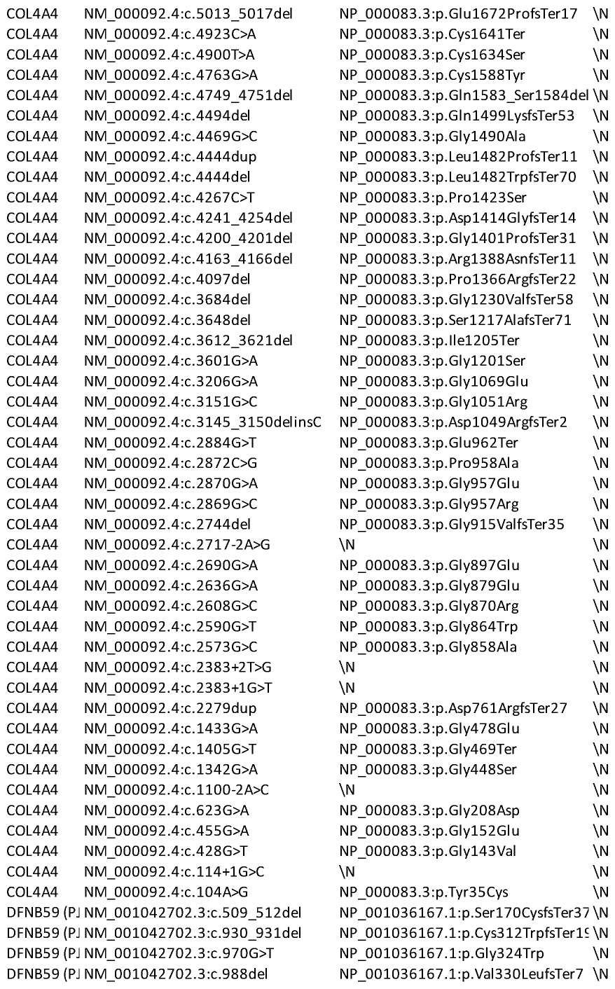

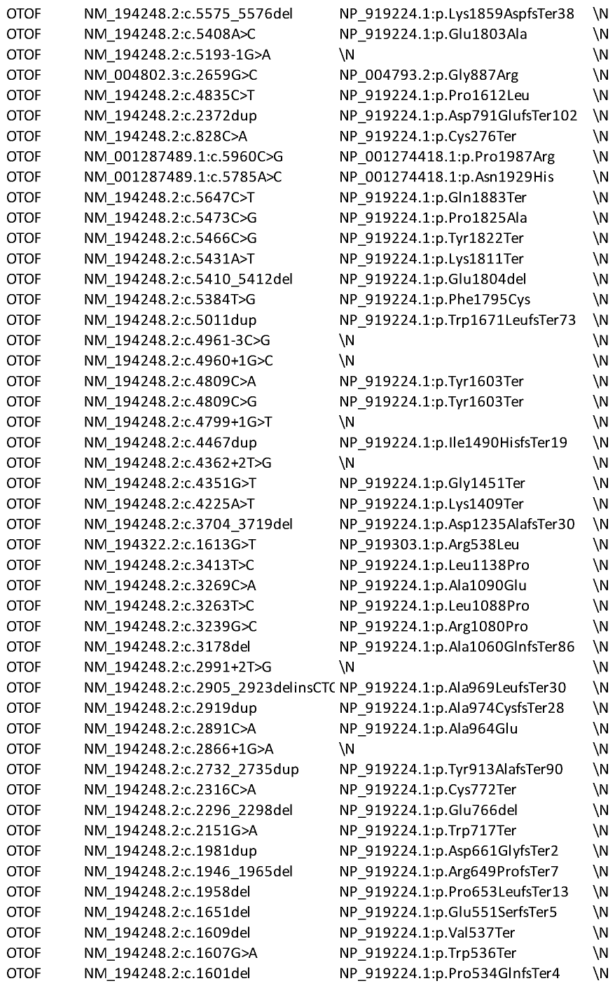

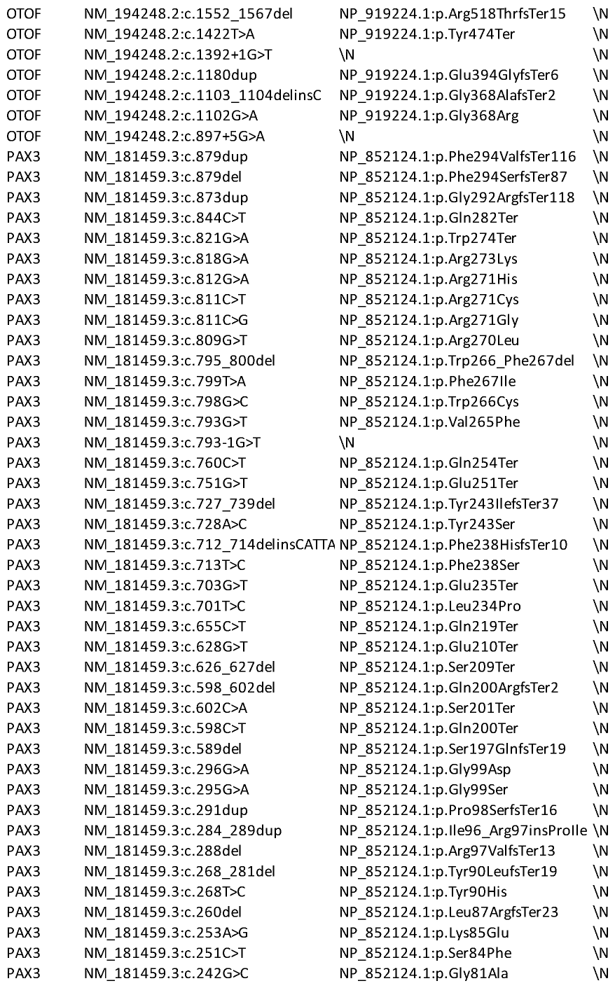

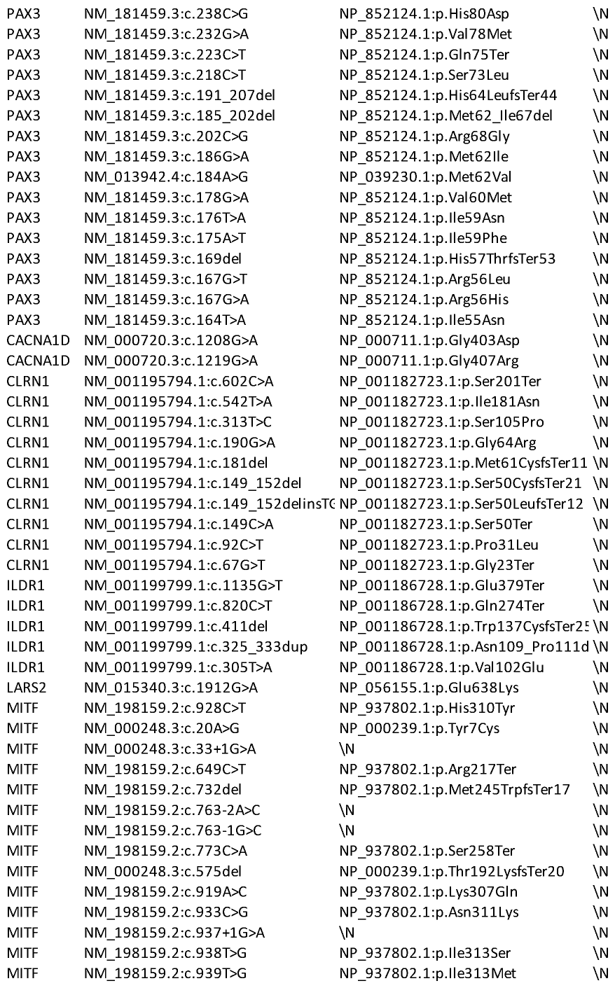

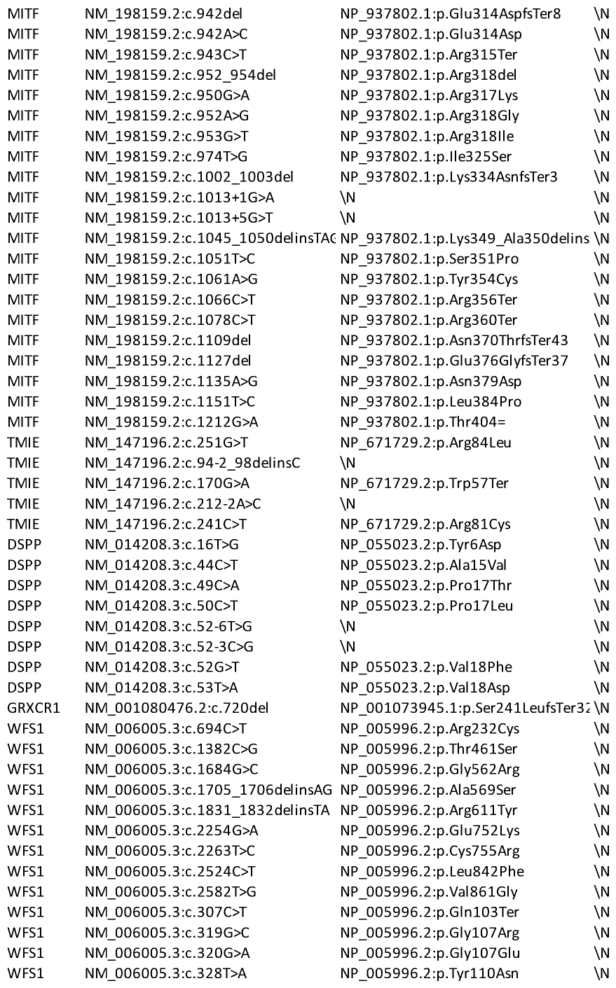

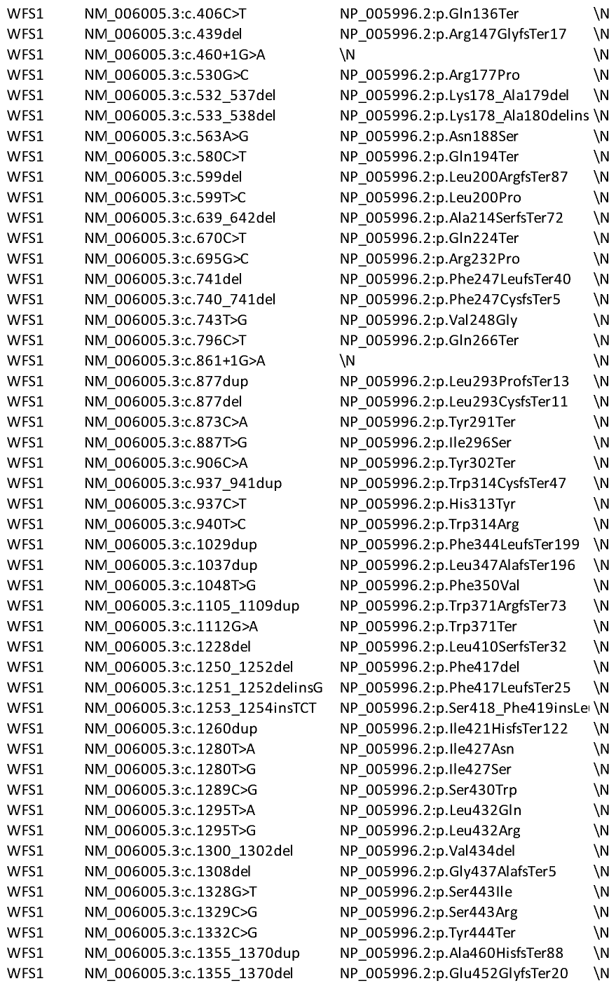

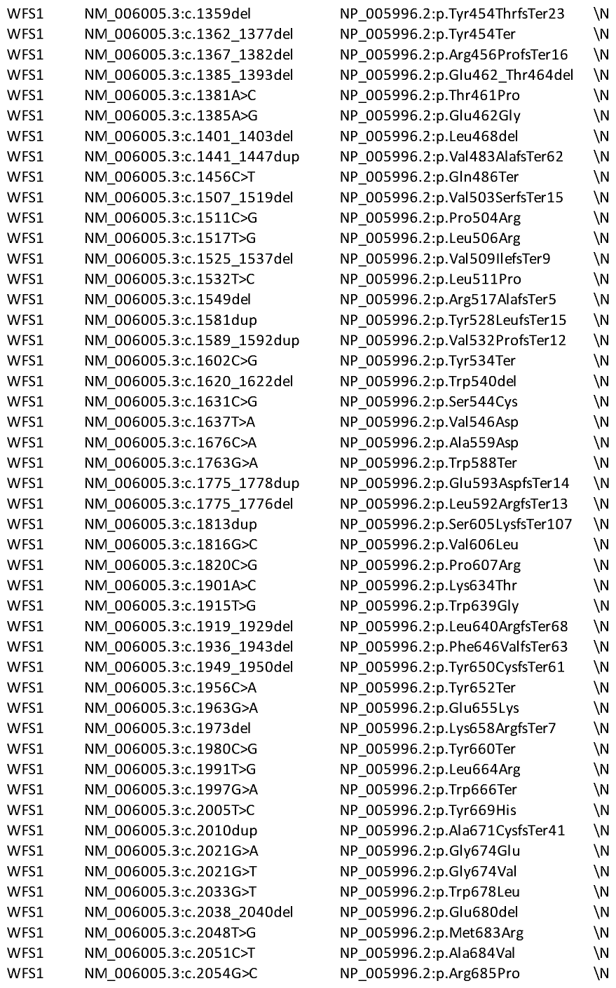

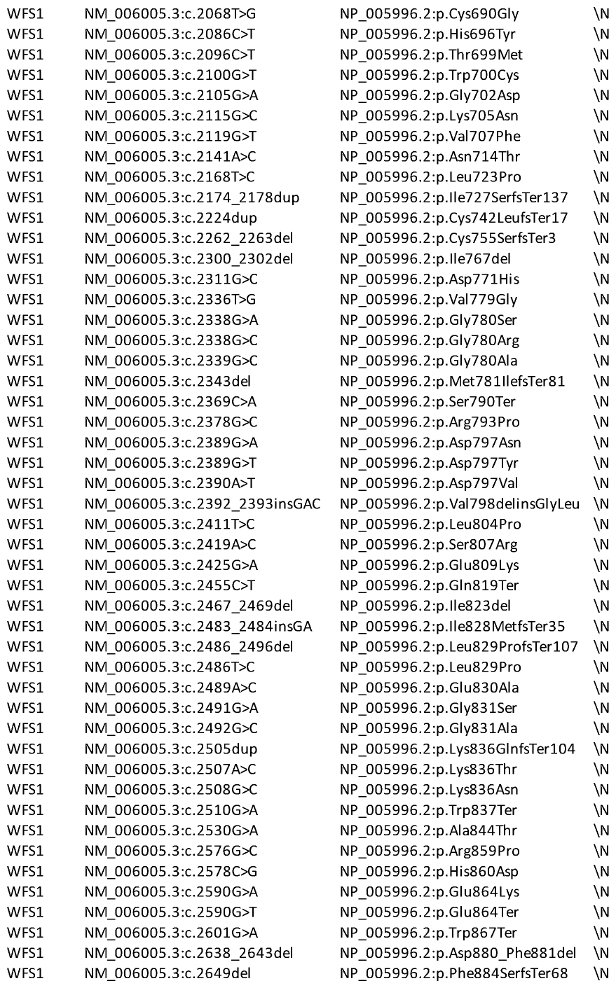

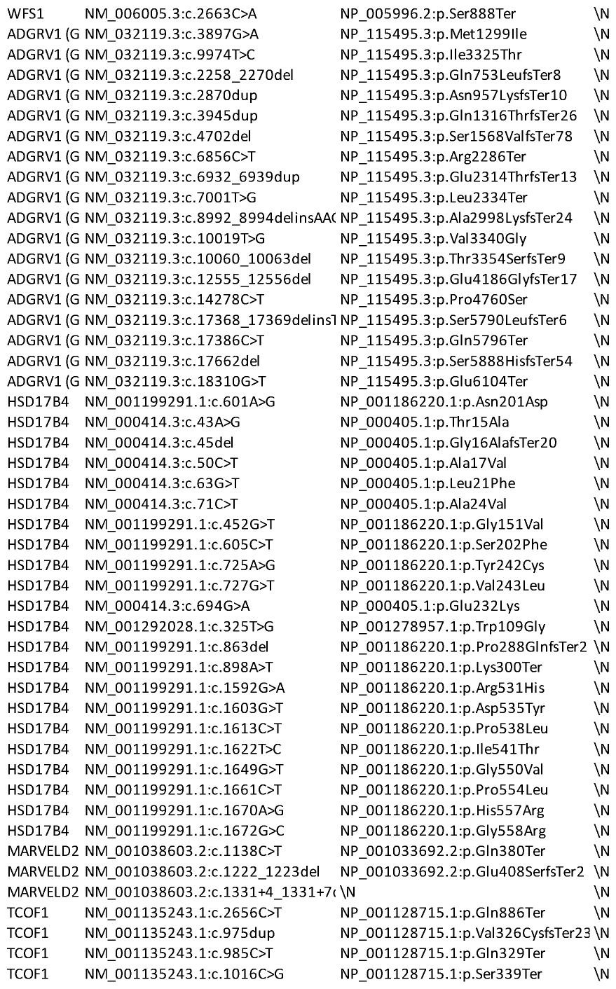

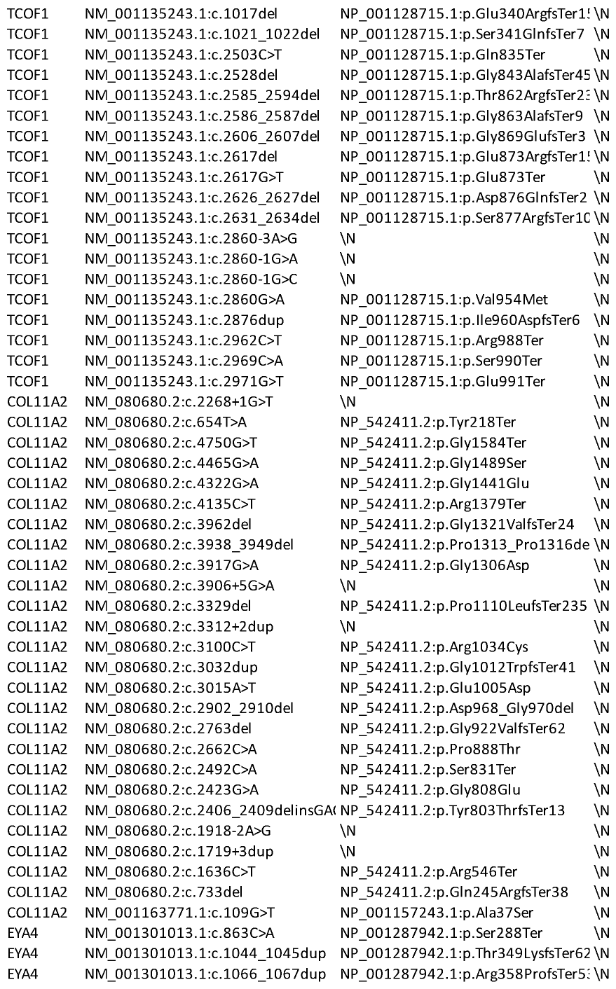

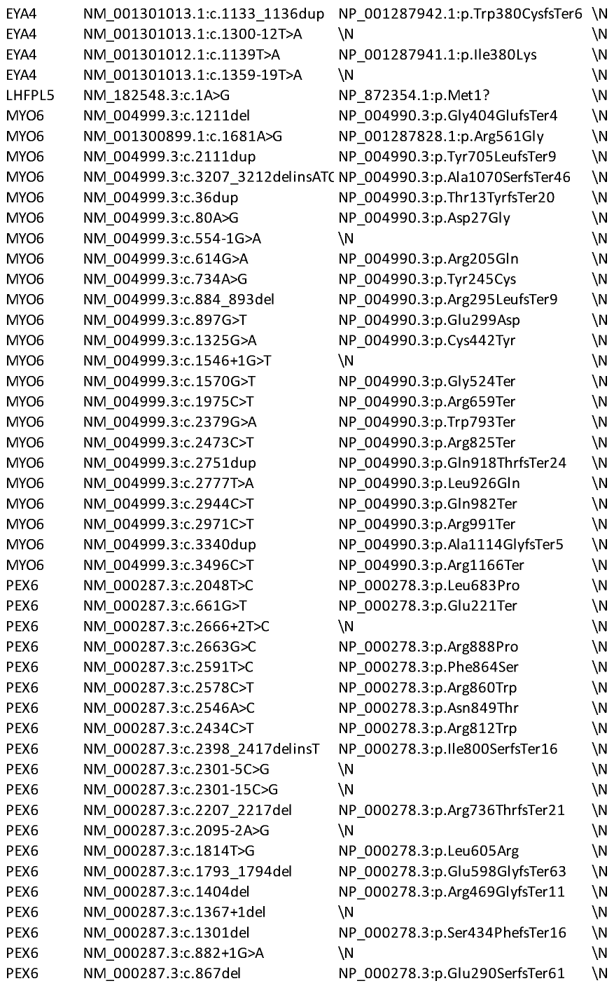

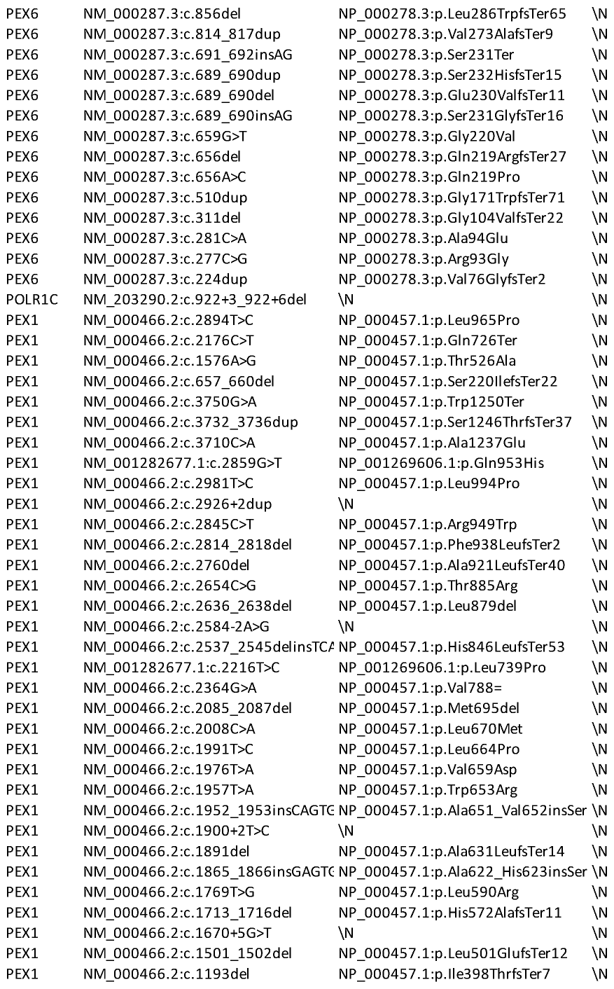

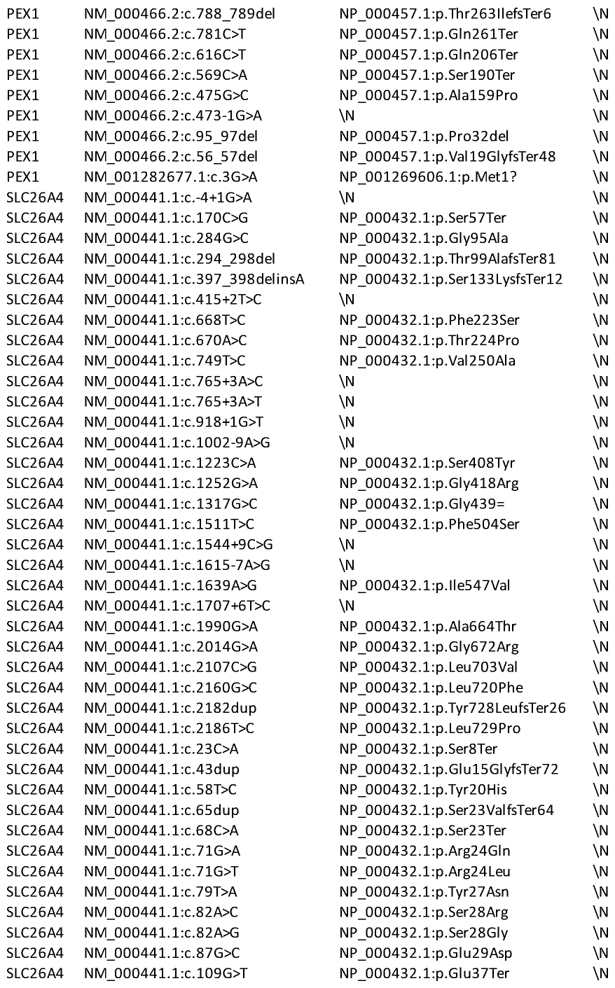

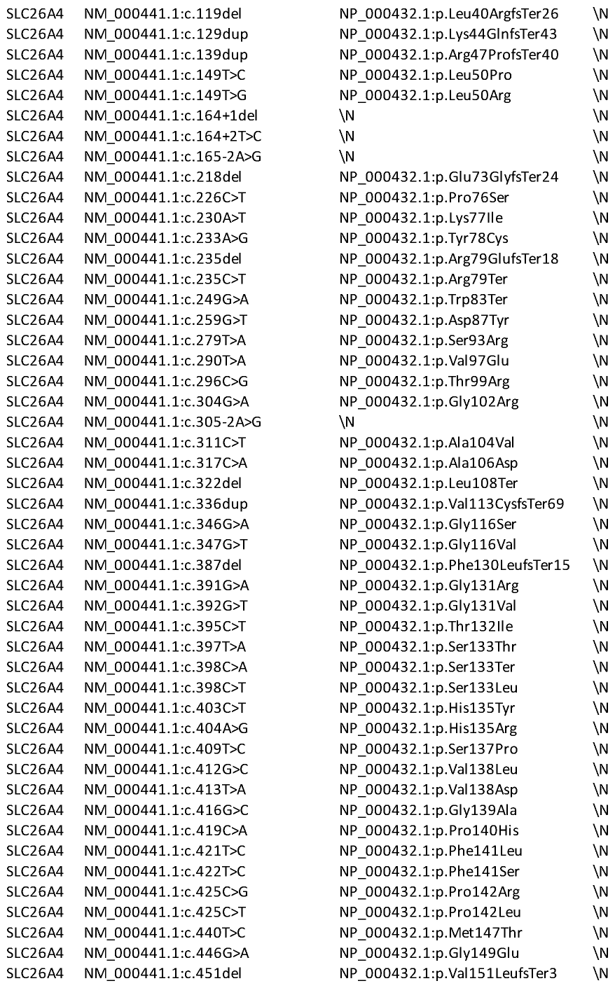

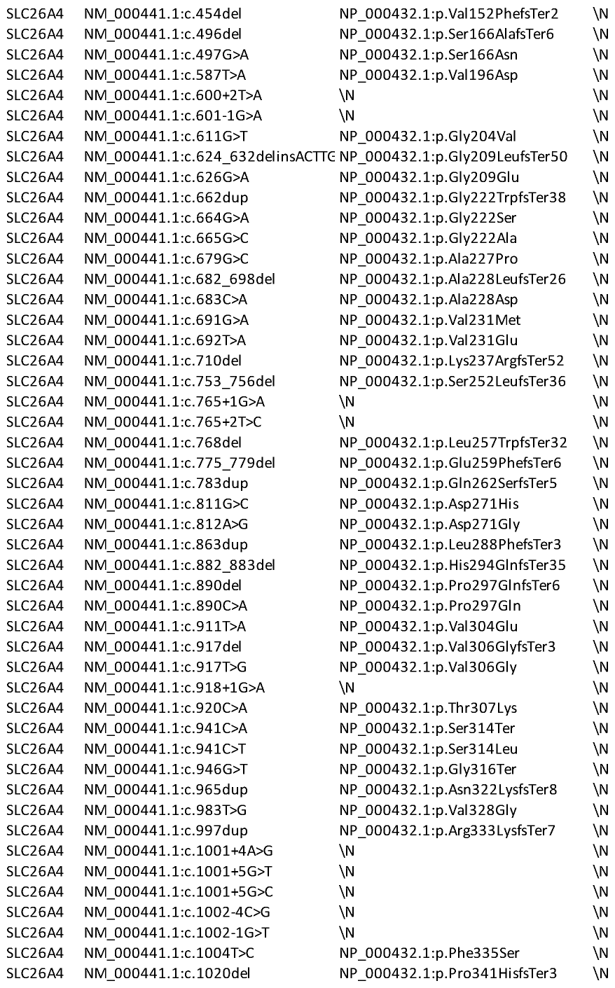

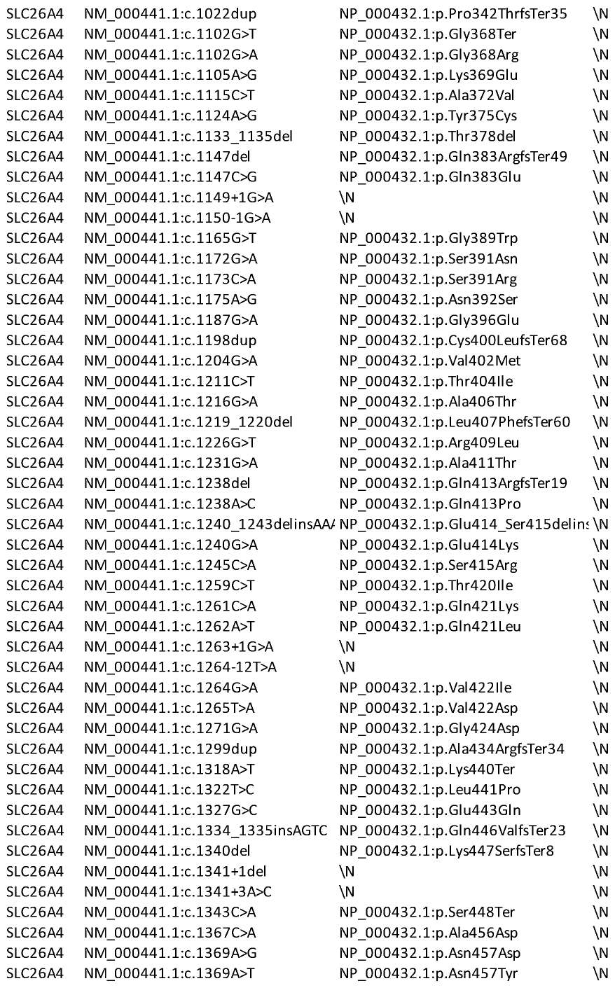

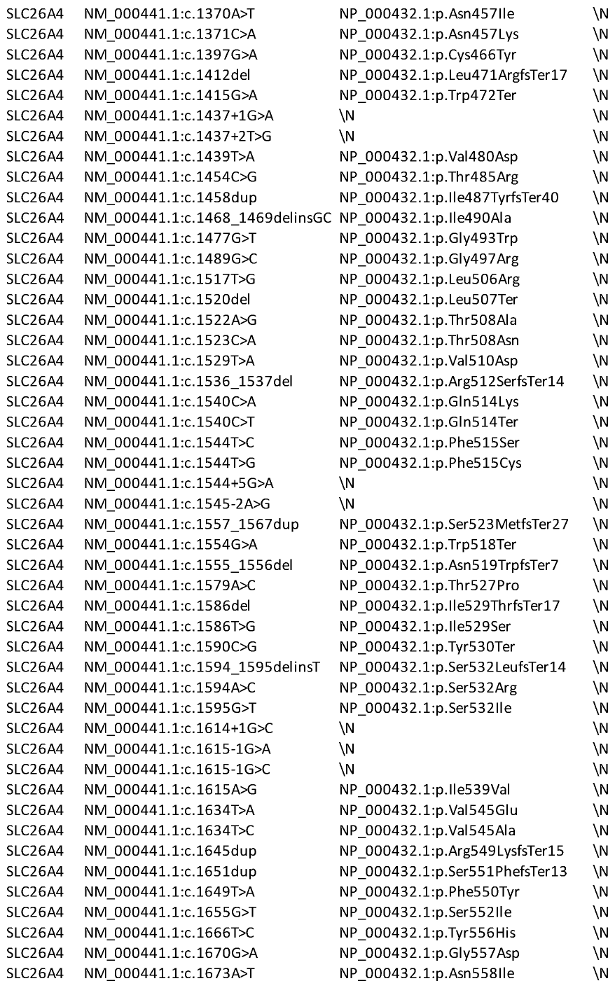

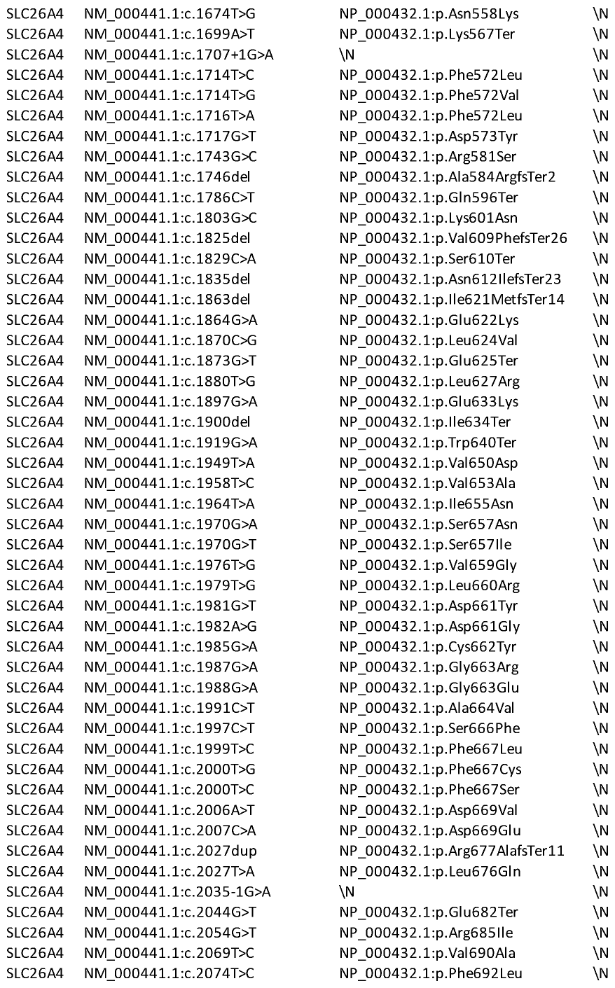

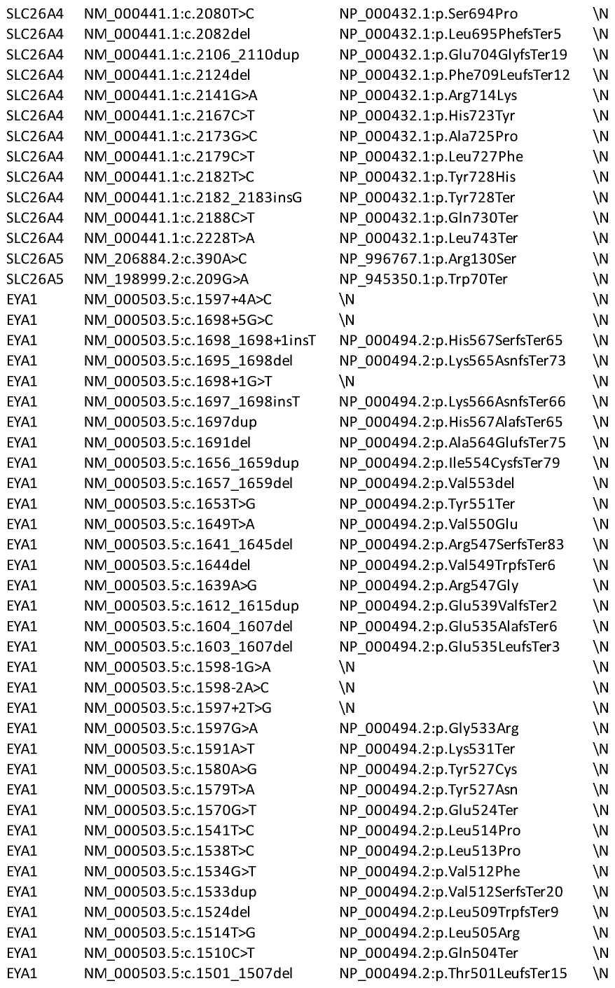

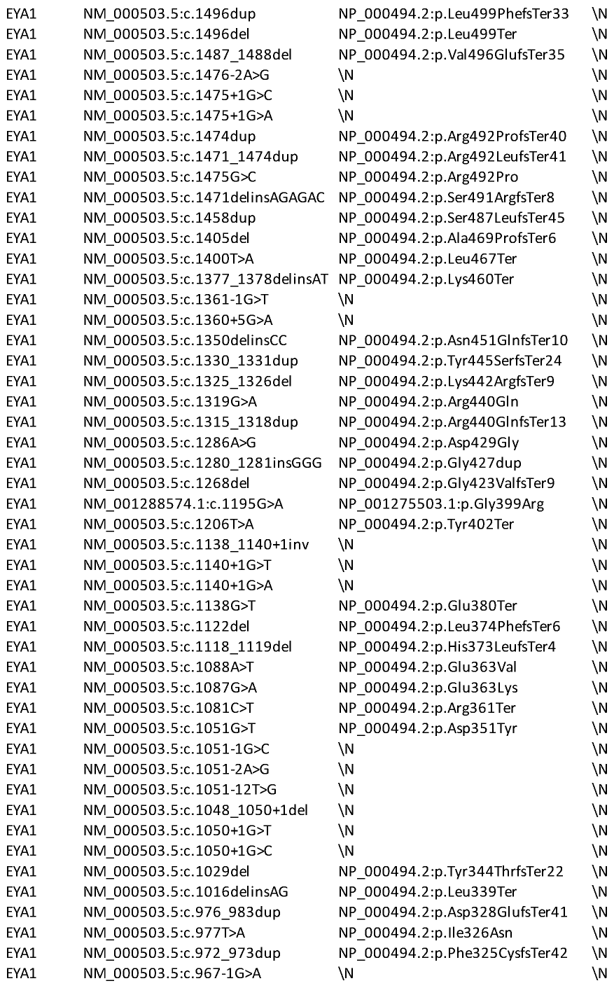

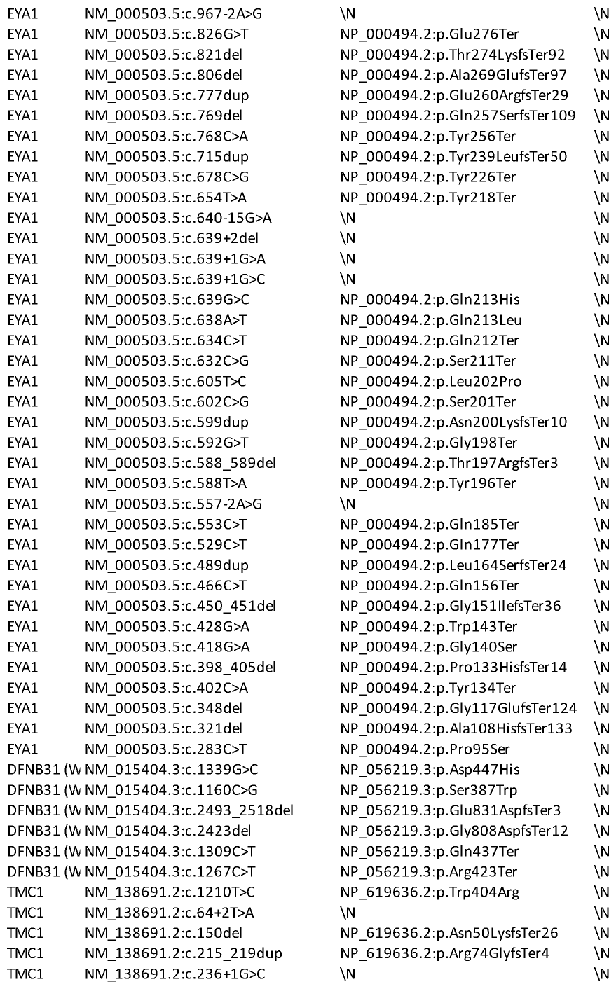

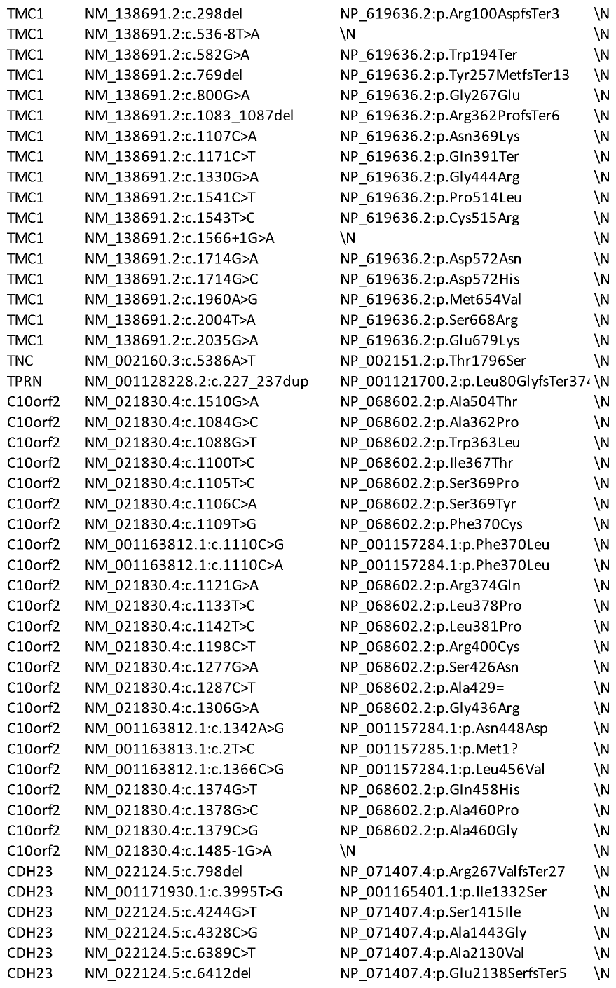

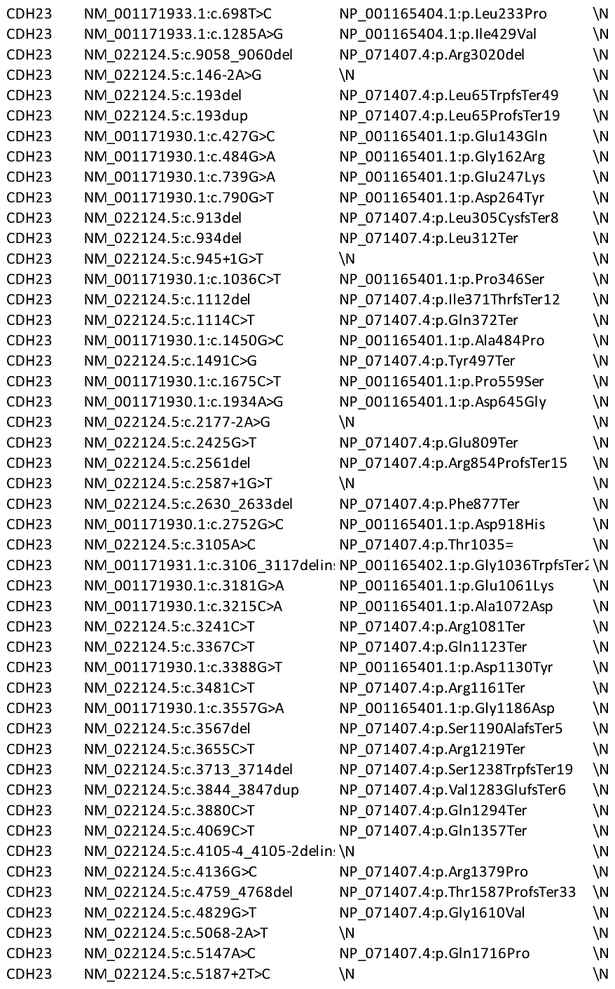

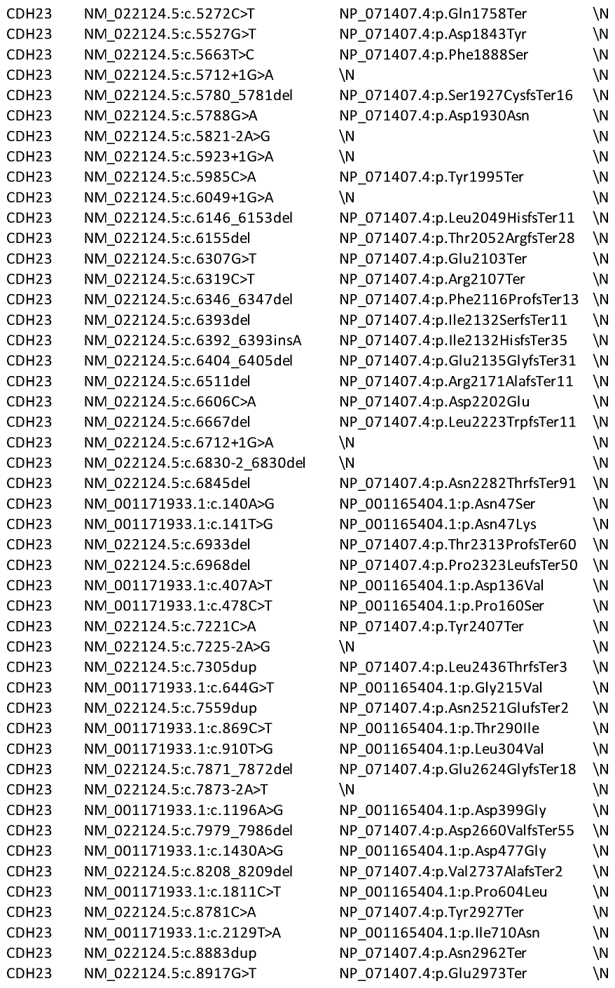

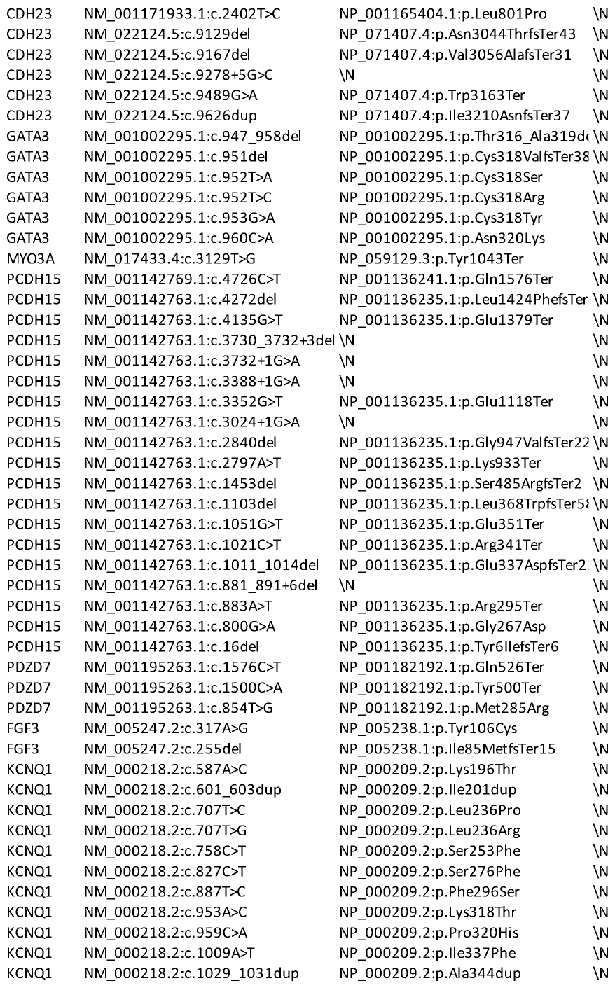

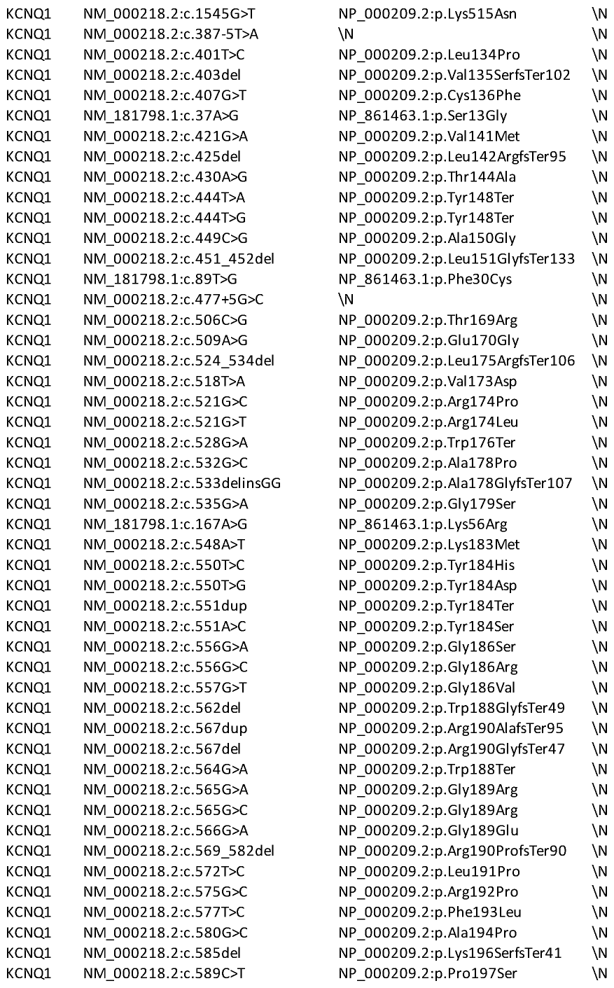

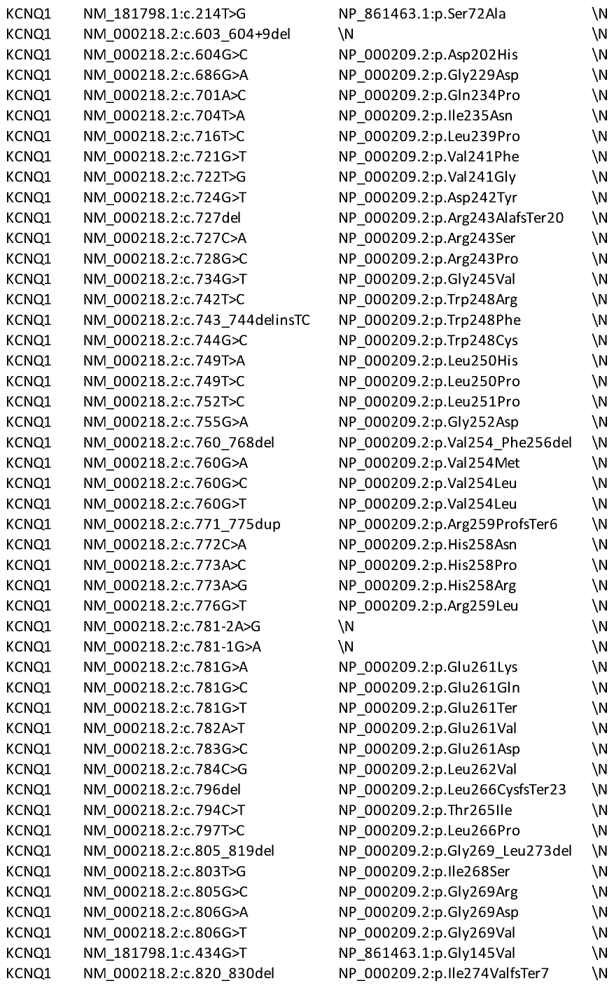

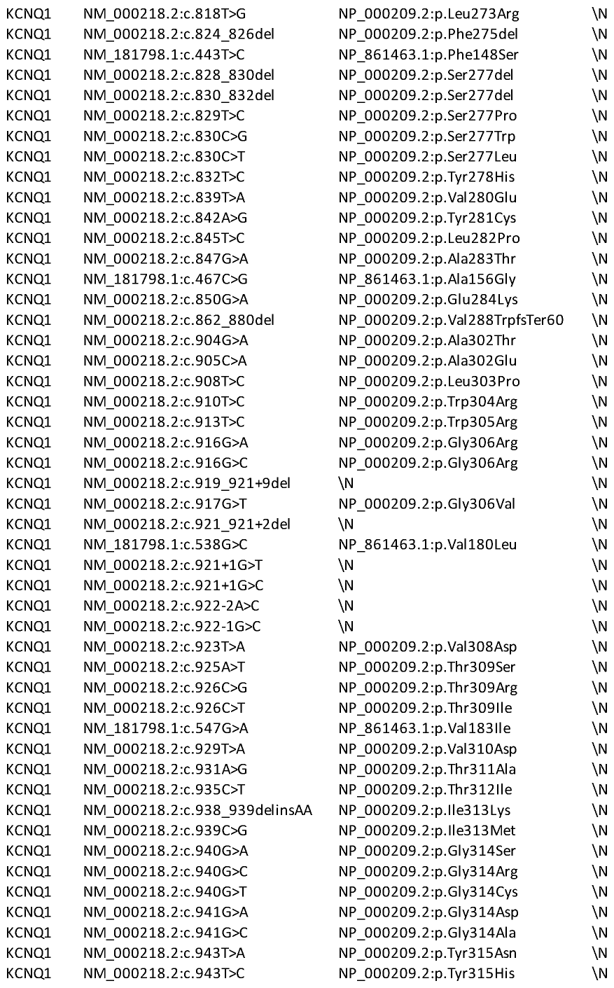

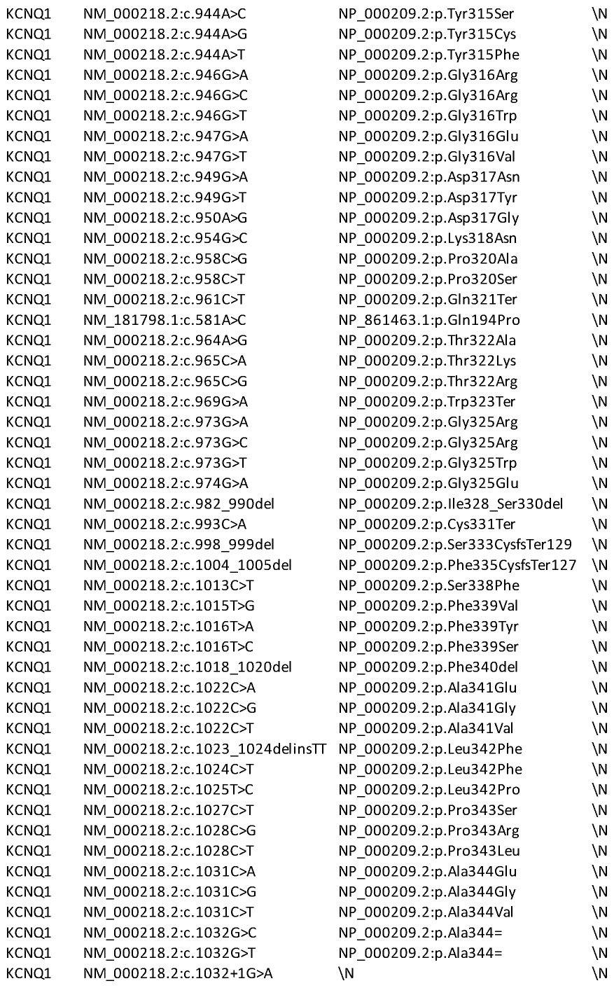

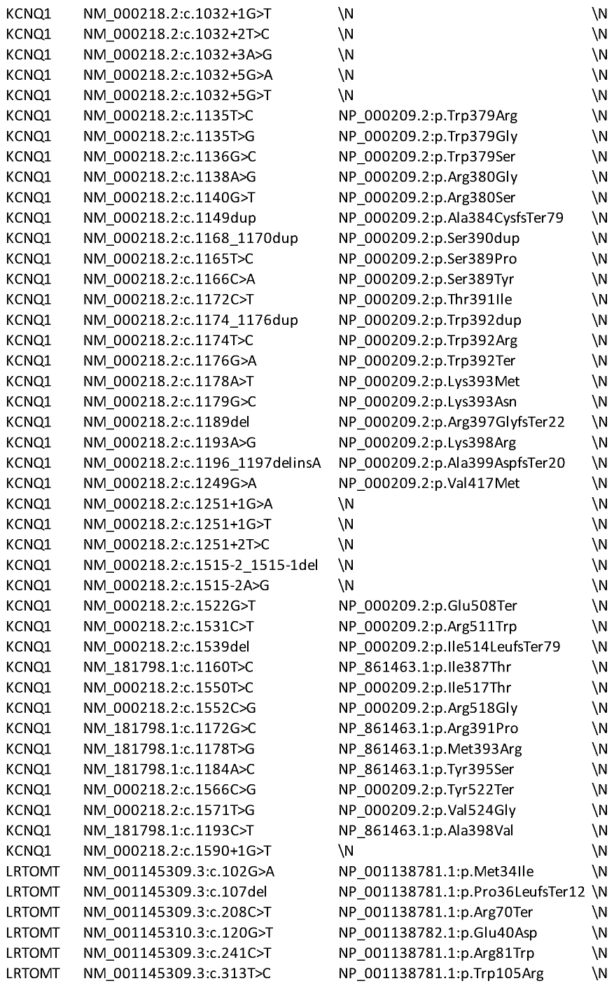

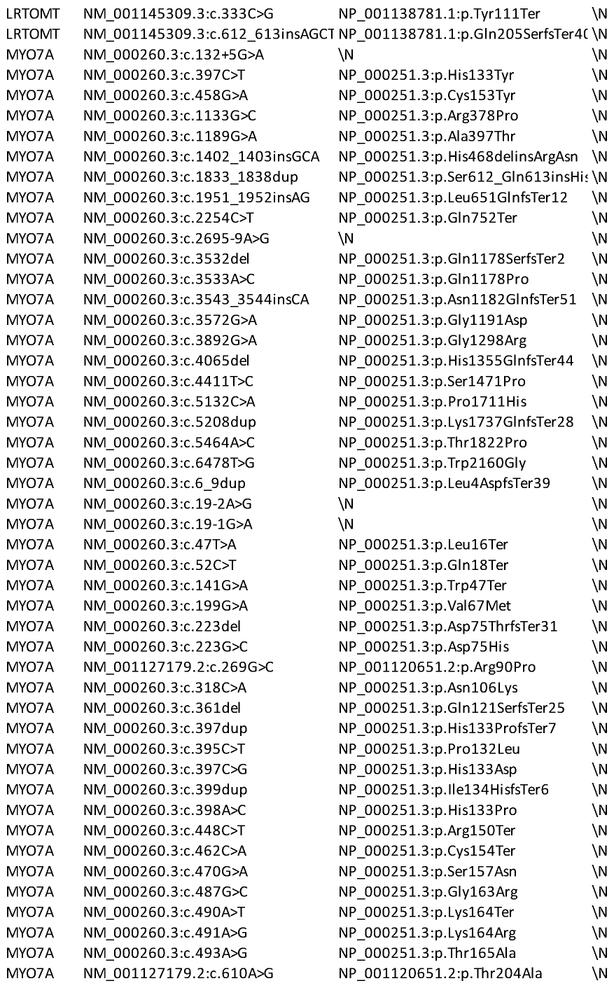

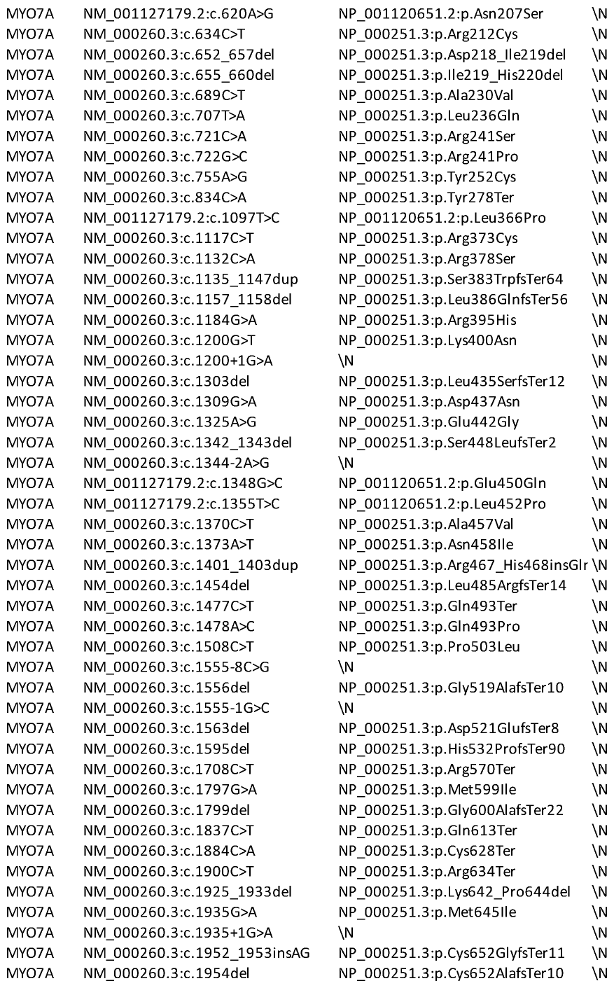

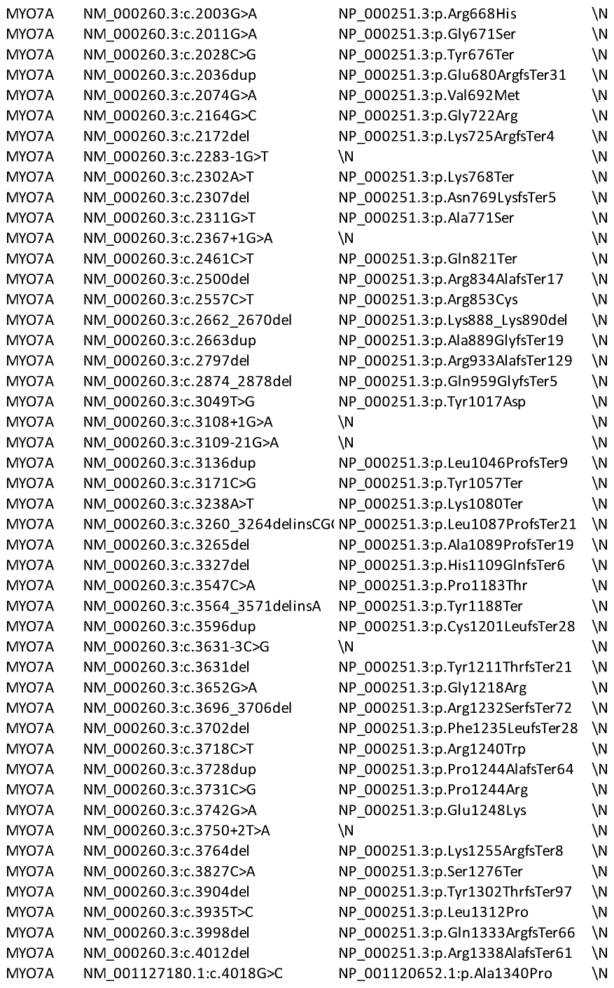

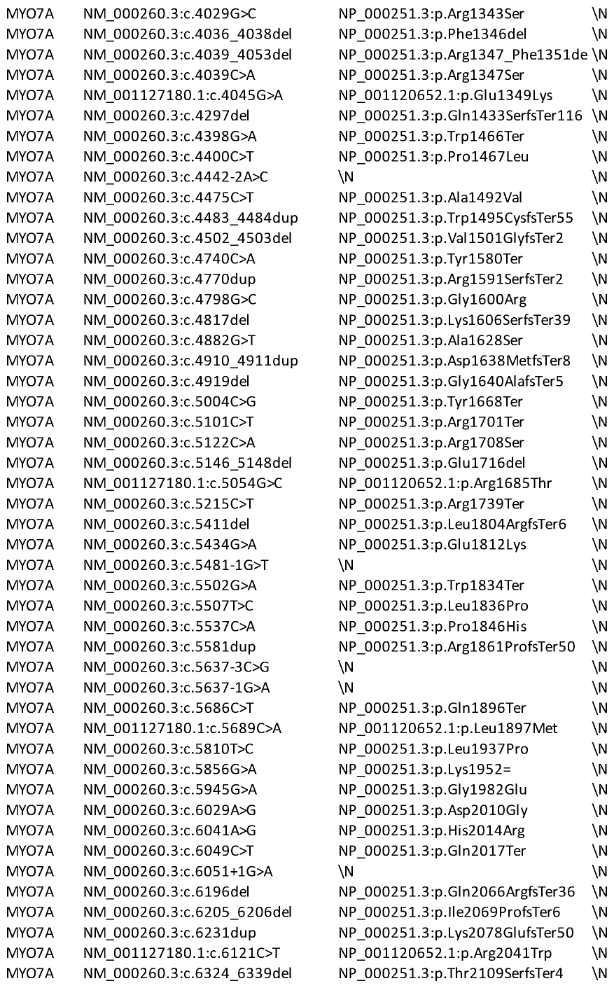

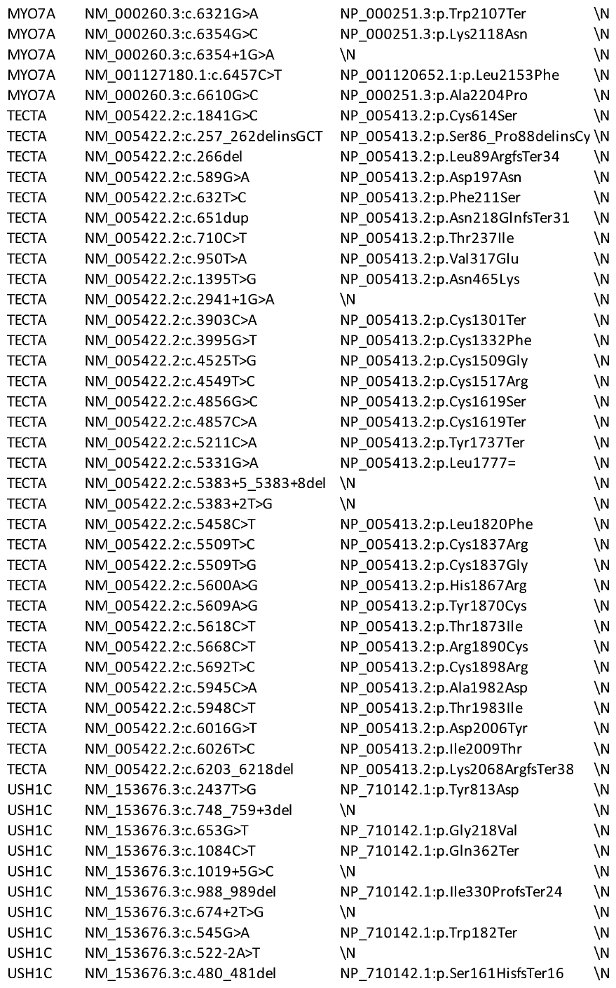

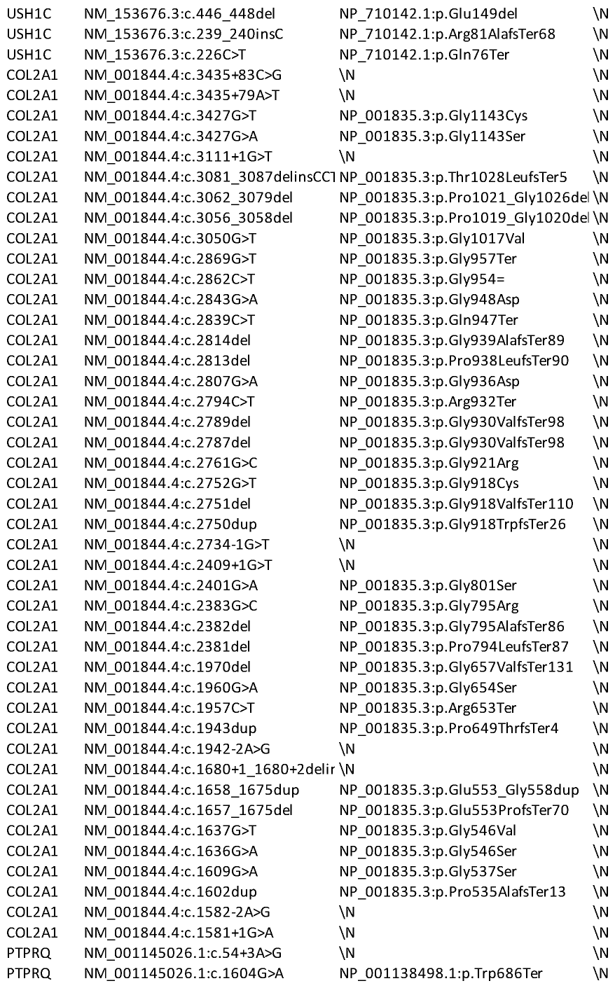

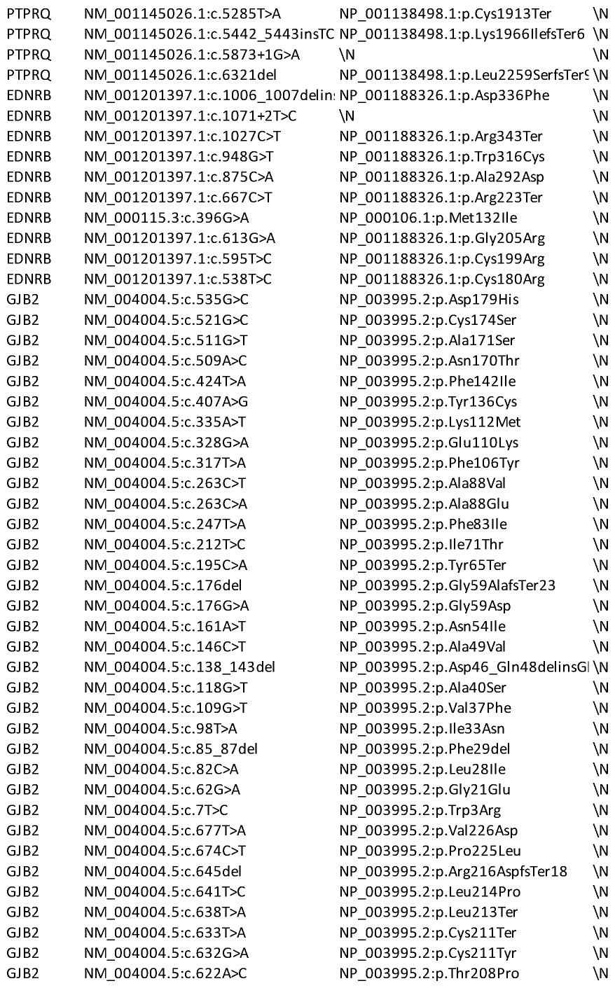

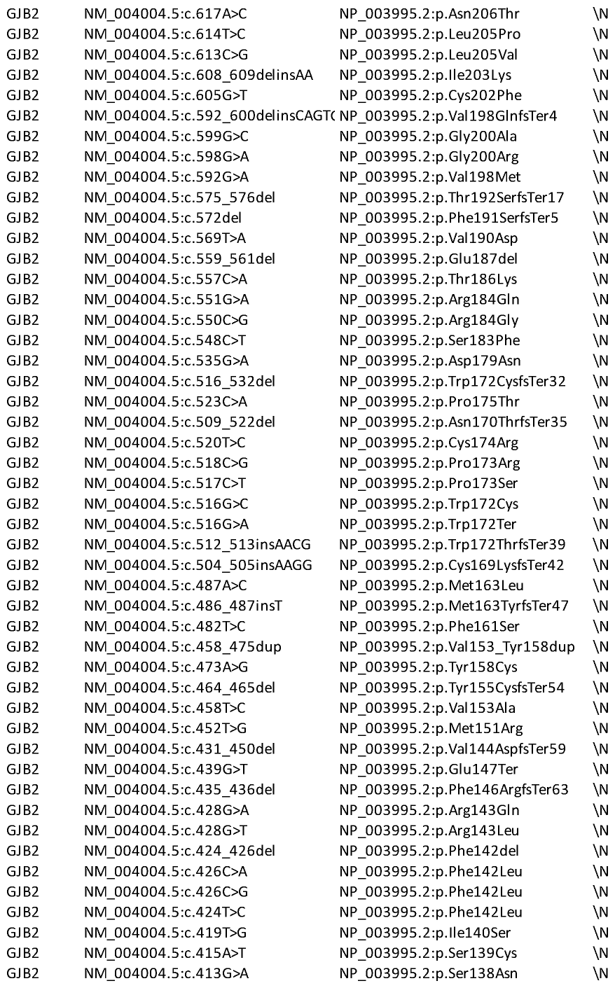

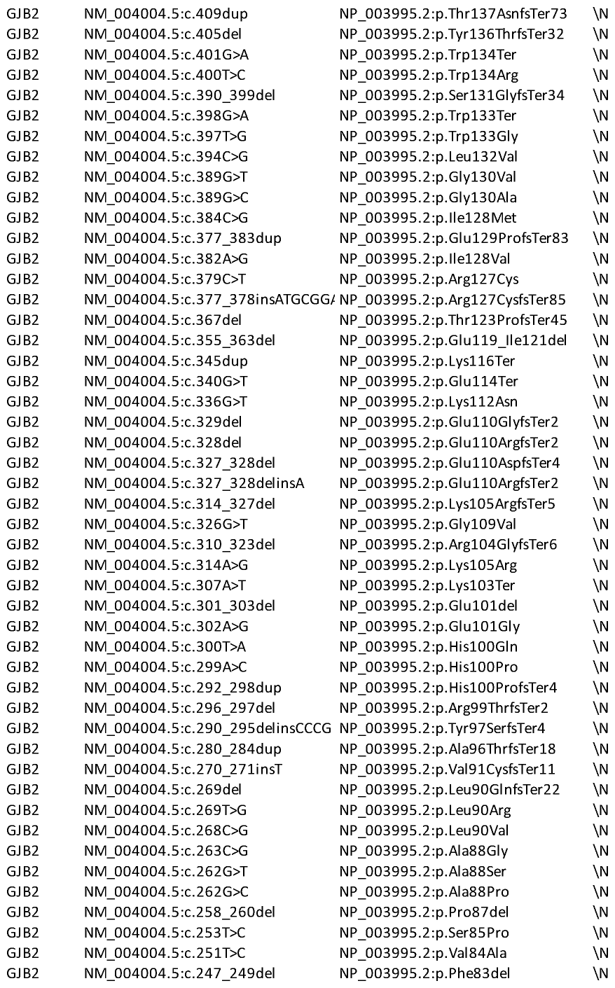

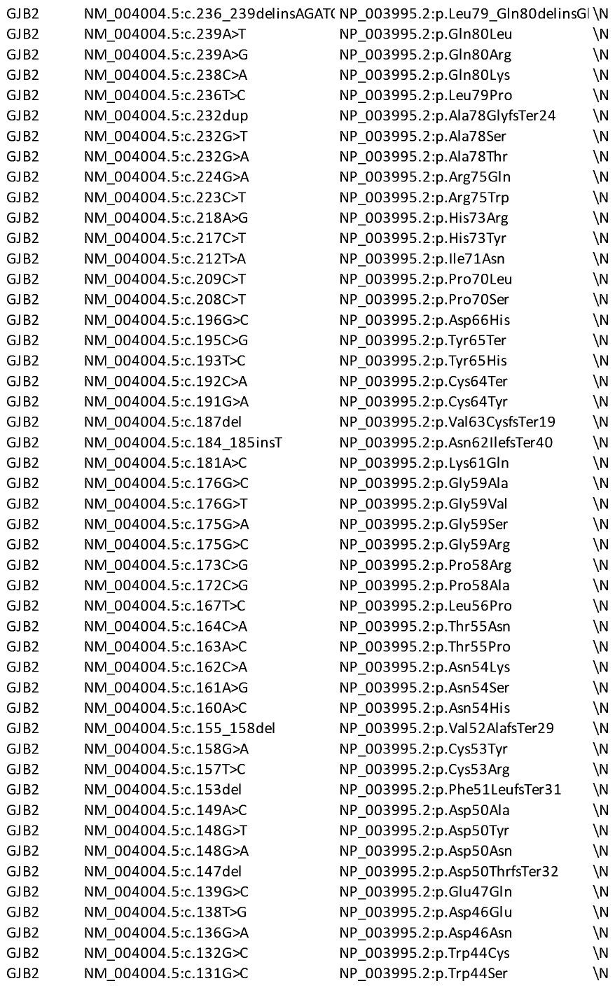

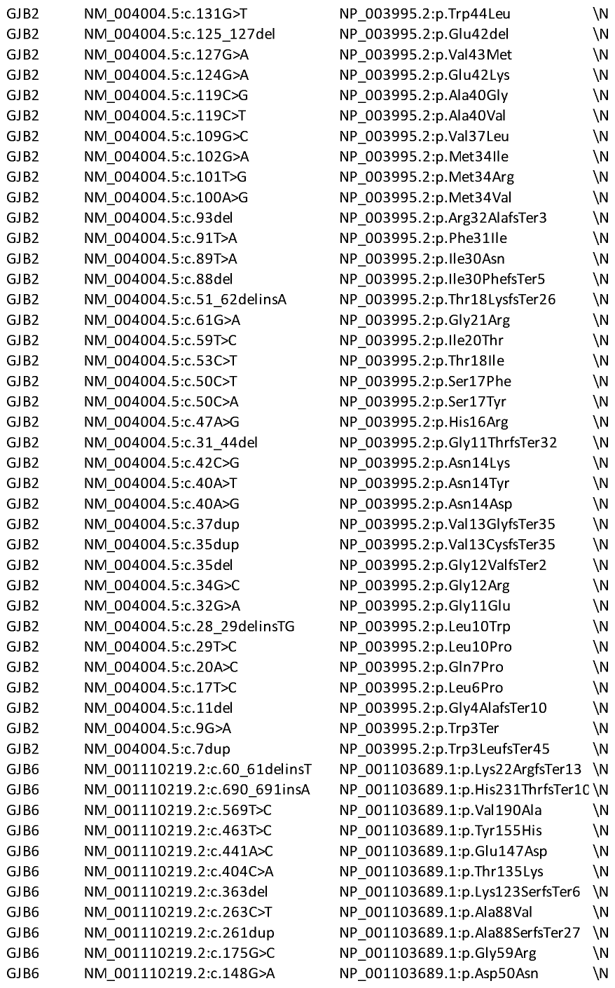

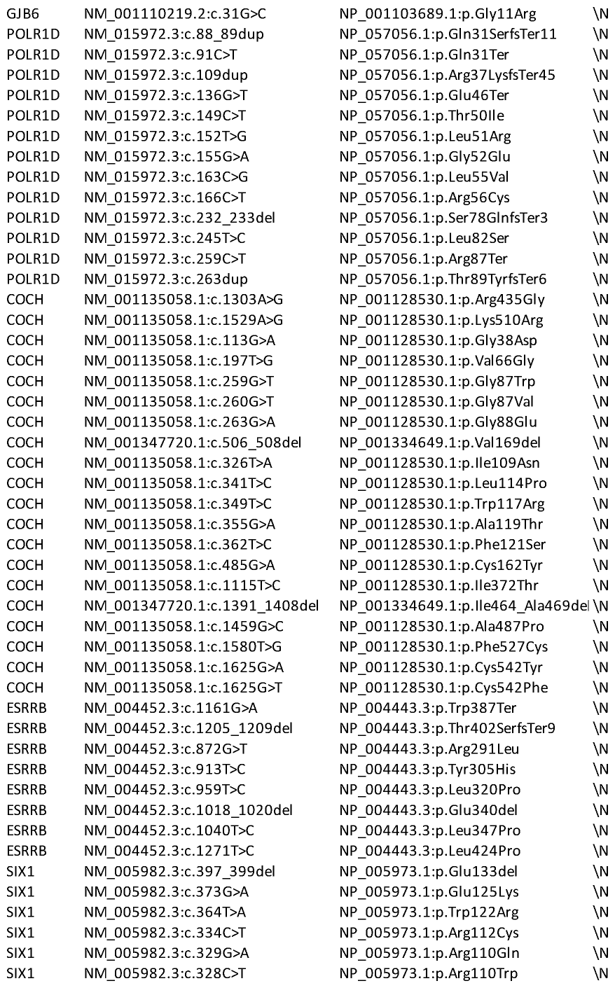

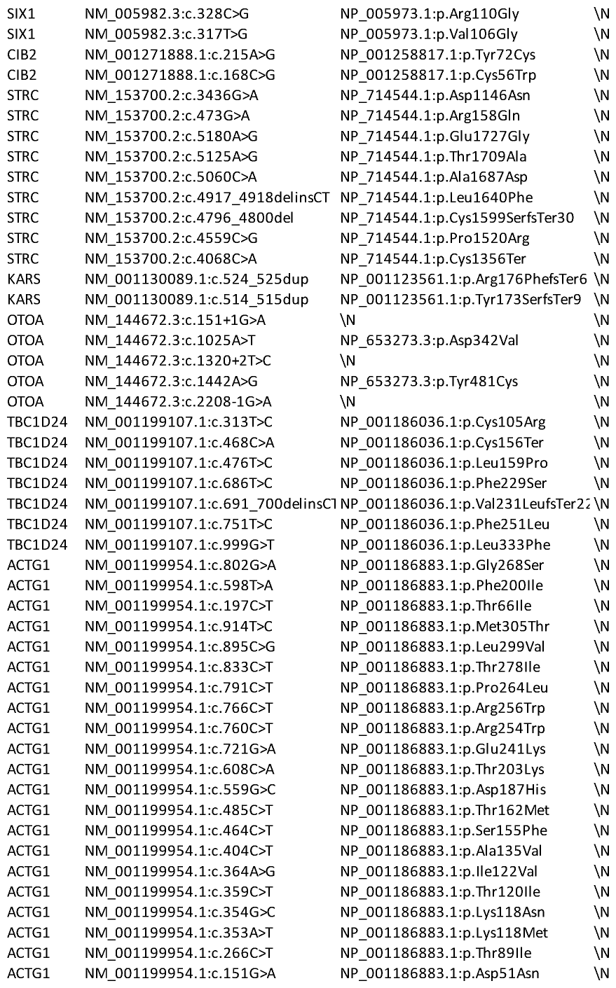

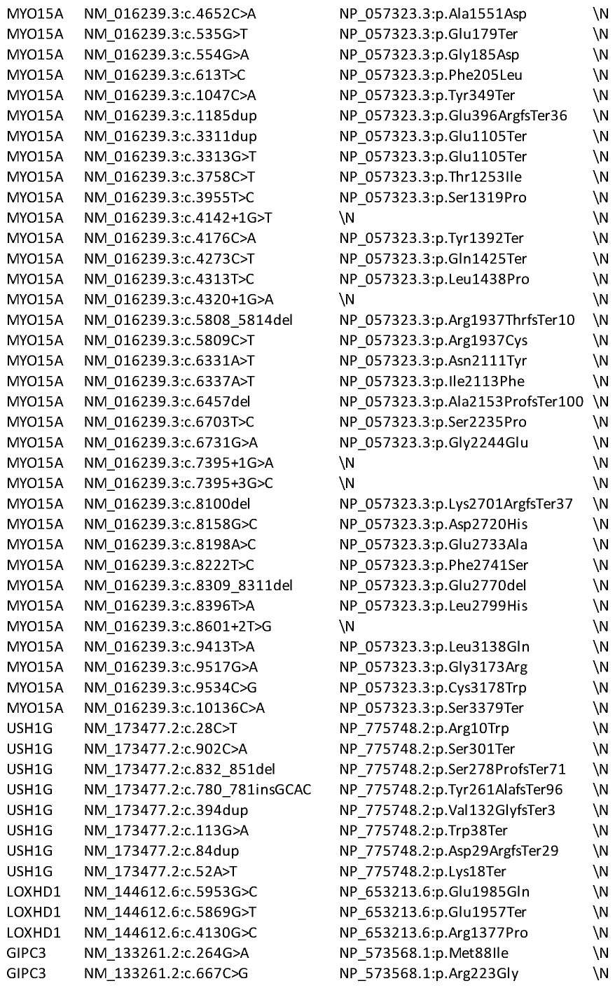

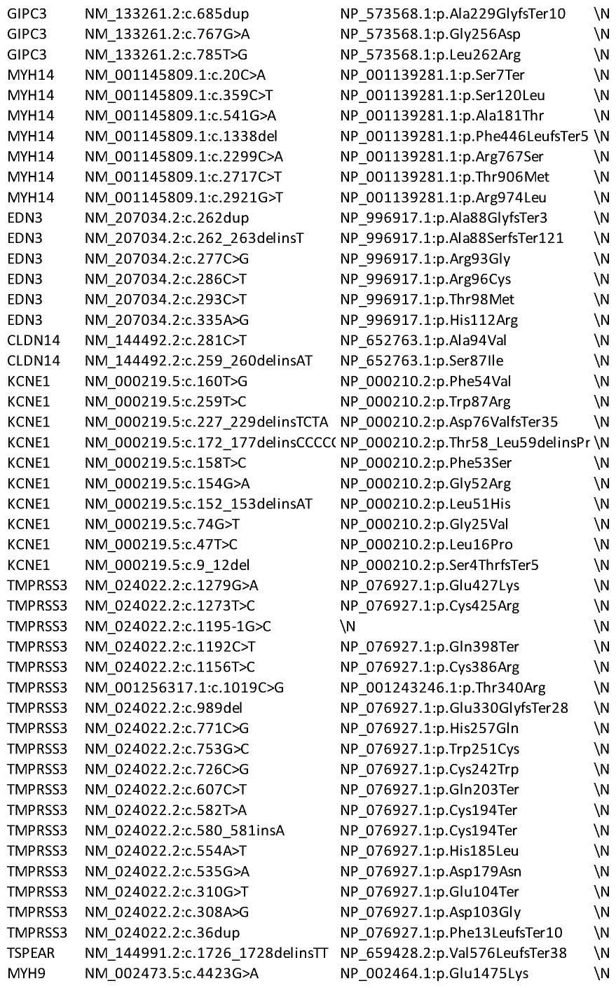

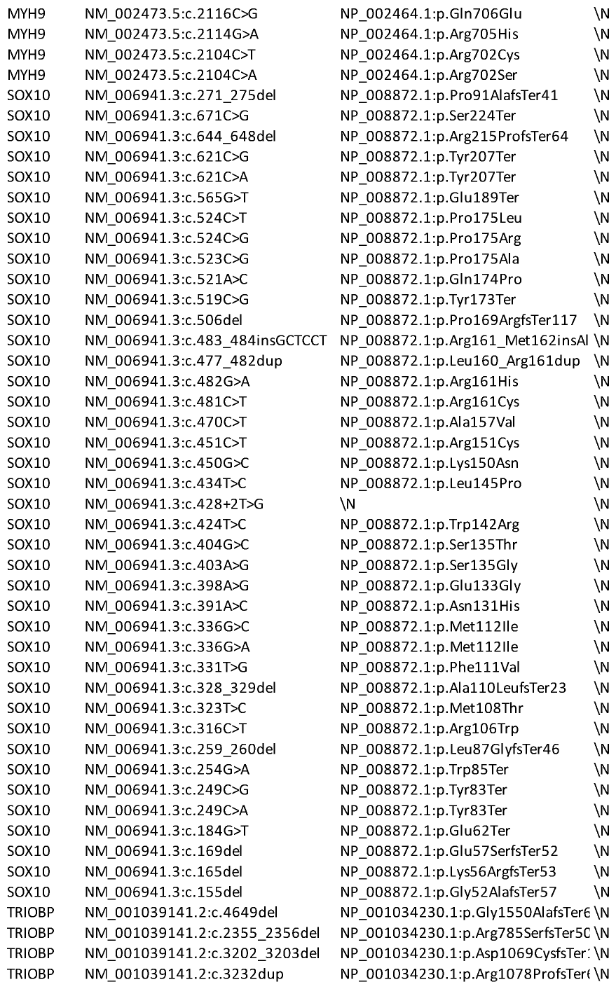

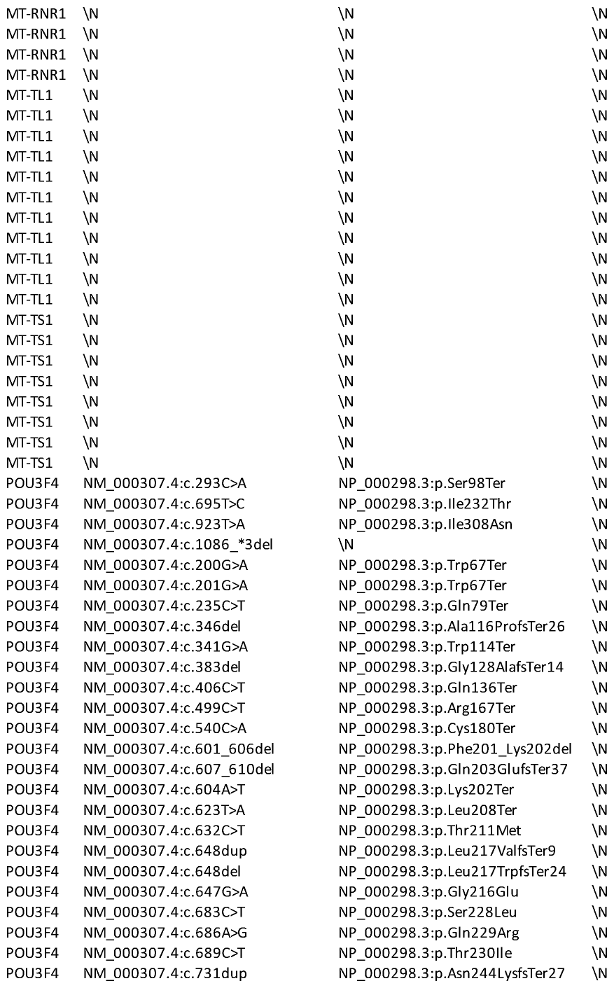

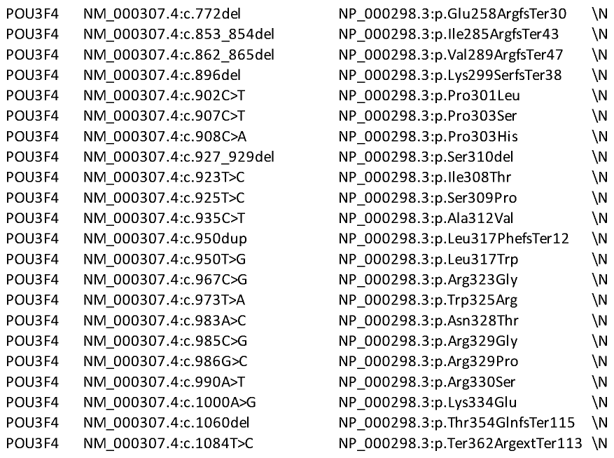

To further increase the diagnostic yield of the panel, variants that were detected more than once in unrelated patients on the OtoSCOPE™ platform (a clinically validated comprehensive genetic panel for the evaluation of hearing loss) were manually included. This led to the addition of 106 additional variants. In total, 1,189 target regions comprising 90,103 base pairs were included in the panel (FIG. 4). At least 4,772 likely pathogenic and pathogenic variants are covered within these regions (FIG. 7).

### Library Preparation, Sequencing, and Analysis

DNA is extracted from whole blood, dried blood spots, saliva, or buccal swabs from individuals following standard protocol. Multiplexed MPS library preparation is performed by running 192 samples in parallel on the Juno™ Targeted Sequencing 192.24 Integrated Fluidic Chip (Fluidigm, San Francisco, CA). Quality control for the MPS library is accomplished using Qubit fluorometric quantification (Thermo Fisher, Waltham, MA). The samples are then pooled and sequenced using the MiSeq platform with a MiSeq Reagent Kit v2 (Illumina, San Diego, CA). Library preparation of the samples may also be performed using standard protocol for targeted comprehensive MPS, exome sequencing, or genome sequencing, though in some instances, this may make the detection of HCMV challenging.

SNVs, CNVs and HCMV are identified on this panel utilizing a custom pipeline on the Galaxy platform (https://usegalaxy.org/) with a combination of proprietary and public open-source software. For identification of SNVs, reads are mapped to the GRCh37 human genome assembly. Initial quality control filtering occurs based on coverage depth, genotype quality and minimum zygosity. Frameshift mutations are realigned to the reference sequence. The final list of these variants is compared with the panel's LP/P variant list. For identification of HCMV, a separate process of quality control filtering occurs after aligning the sample reads to the human cytomegalovirus assembly (strain Merlin). For samples in which amplification of human cytomegalovirus regions occurs, the viral read depth is compared to the read depth of the human genomic targets to estimate viral load. For identification of CNVs, a depth-of-coverage approach based on comparison to pooled samples is used. In most cases, the breakpoints of CNVs will not lie within the amplified regions. By utilizing a machine learning-based approach based on a training dataset of patients previously diagnosed with CNVs in the genes of interest, the process of calling CNVs is automated.

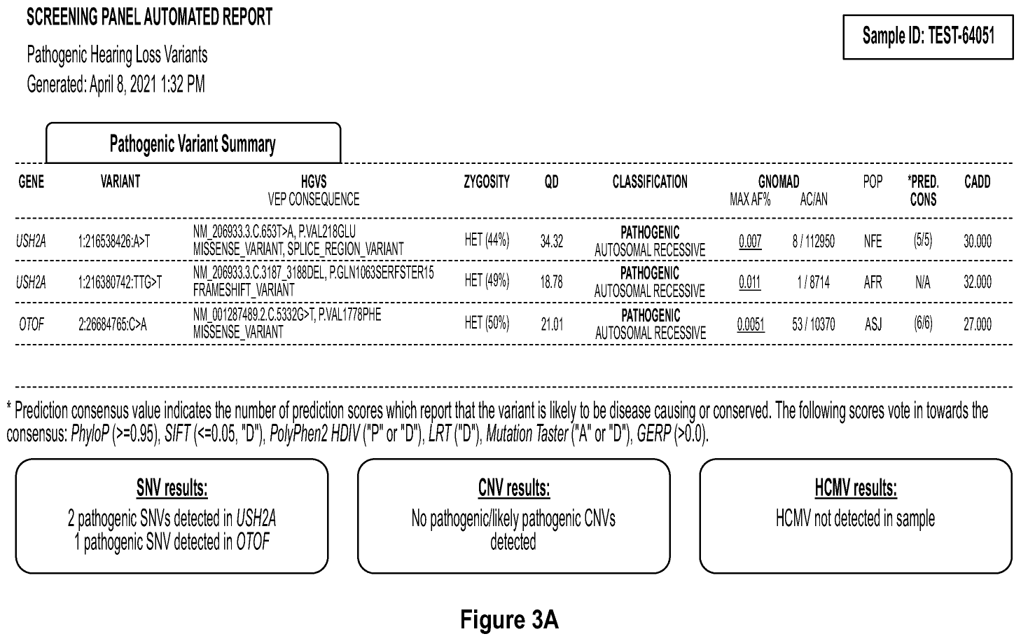

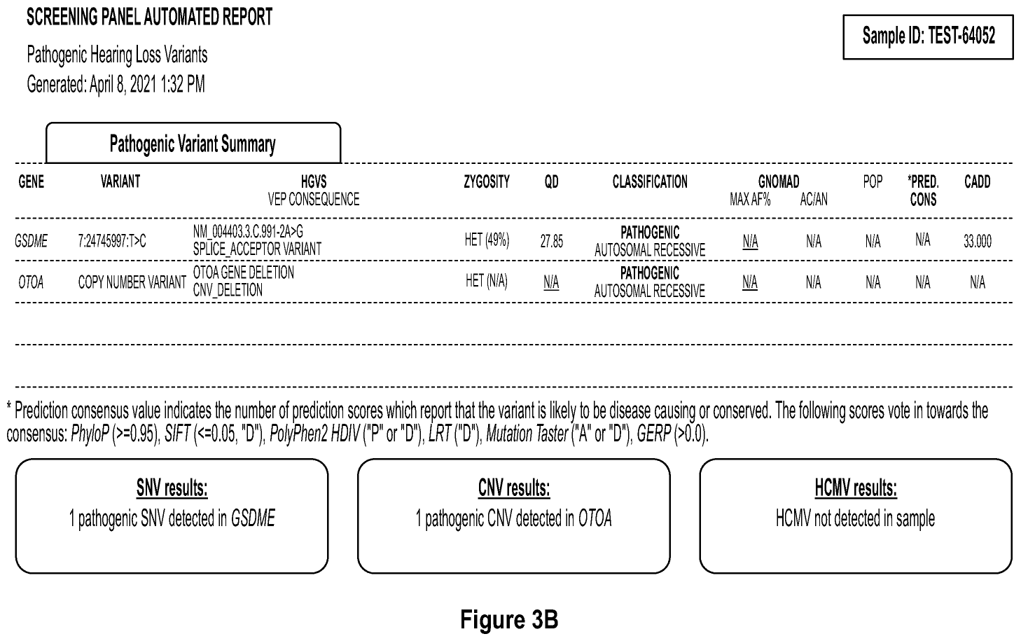

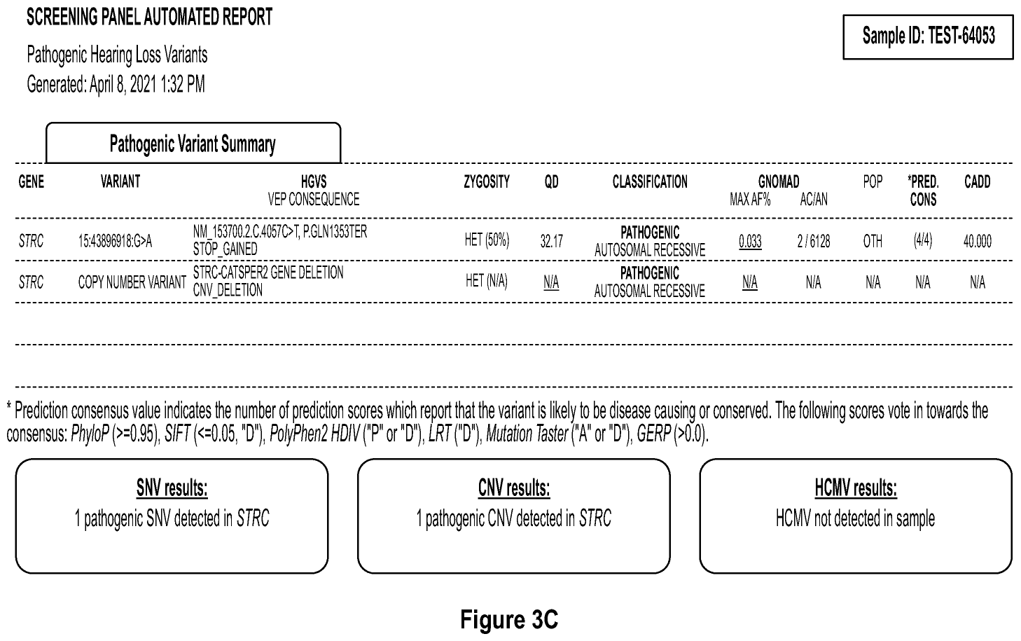

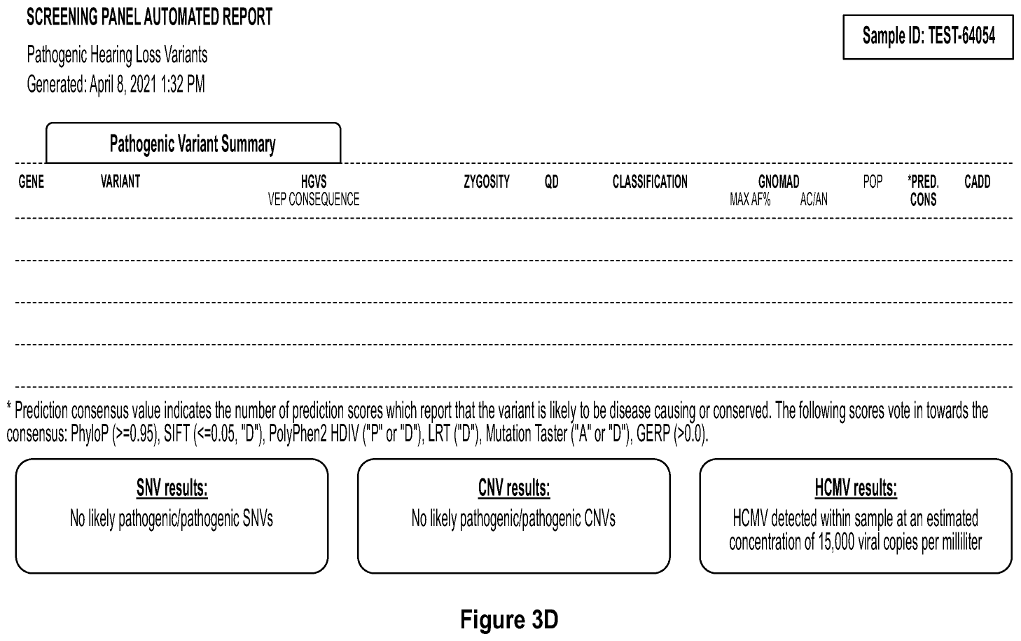

Using a graphical report with data visualization, the pipeline collates the result of the SNV, CNV and HCMV analysis from a FASTQ file and automatically generates a report for both genetic hearing loss and HCMV. The genetic hearing loss portion calls a result for the presence of LP/P variants and their inheritance pattern for the SNVs and CNVs from the sequencing data. HCMV presence or absence and estimated viral load is also reported. If sample quality is inadequate based on the pipeline's filtering, a result of “unable to analyze secondary to insufficient sample quality” is returned. All identified LP/P variants are included in the final report. An example of this output is presented in FIG. 3. In FIG. 3A, two pathogenic variants within the USH2A gene were identified. This finding indicates a presumptive diagnosis of autosomal recessive hearing loss. A single pathogenic variant was detected in the OTOF gene, which indicates the patient is a carrier of a pathogenic variant in this gene. No other LP/P variants were found in any of the panel's target regions. In FIG. 3B, a single pathogenic variant was detected in GSDME, which indicates a presumptive diagnosis of autosomal dominant hearing loss. A CNV was detected in OTOA, which indicates the patient is a carrier of a pathogenic variant in this gene. In FIG. 3C, an SNV and CNV were detected in STRC, which indicates a presumptive diagnosis of autosomal recessive hearing loss. In FIG. 3D, the presence of human cytomegalovirus was noted, and the number of viral copies per milliliter was estimated; further interpretation of this result depends on patient age and clinical characteristics. Based on an analysis of 1,524 samples that received a positive diagnosis of autosomal recessive hearing loss via comprehensive massively parallel sequencing panel (OtoSCOPE™), this method would correctly identify the genotype in 1,223 (80.2%) samples as a first-tier test. If a definitive positive genetic diagnosis was not made but a single variant pathogenic or likely pathogenic for autosomal recessive hearing loss was identified by the panel, reflexing to a comprehensive massively parallel sequencing panel would allow for capture of an additional 187 (12.3%) of diagnoses. This tiered approach offers a positive predictive value far superior to typical physiologic hearing screening and will result in significant cost savings for patients seeking genetic testing.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range. As used herein, the term “about” means±10%.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

1. A method of detecting a biomarker in a patient's genome comprising:
(a) obtaining nucleic acid sequence data of the patient's genome,
(b) processing the sequence data to generate a patient dataset for the patient, and
(c) comparing the patient dataset to a biomarker panel, and
(d) generating an automated patient report based on biomarkers that are present in the patient dataset,
wherein the patient report identifies one or more biomarkers present in the patient dataset and/or copy-number variants present in the patient dataset, and
wherein the one or more biomarkers are genetic variants or genomic target regions associated with nonsyndromic genetic hearing loss, nonsyndromic genetic hearing loss mimics, selected genetic syndromes with hearing loss as a major feature, or infection-induced hearing loss.

2. The method of claim 1, wherein the patient report is a fully automated report.

3. The method of claim 1, wherein the generating of the patient report is performed in less than about two weeks from the time of sample receipt, and in the timeframe of hours for the data analysis portion.

4. The method of claim 1, wherein the automated patient report is generated by a computer.

5. The method of claim 1, wherein the patient dataset is a Massively Parallel Sequence (MPS) FASTQ file.

6. The method of claim 5, wherein the MPS FASTQ file was generated from a genome sequence, an exome sequence, a targeted amplicon-based library preparation or a deafness-targeted gene panel (such as OtoSCOPE™).

7. The method of claim 1, wherein the patient dataset is a panel of target regions.

8. The method of claim 1, wherein the biomarker panel comprises at least 96 biomarkers associated with non-syndromic hearing loss.

9. The method of claim 1, wherein the biomarker panel comprises at least 4,000 biomarkers.

10. The method of claim 9, wherein the biomarkers associated with nonsyndromic hearing loss, nonsyndromic hearing loss mimics, and selected syndromes with deafness as a major feature are identified from a group of over 200 genes associated with hearing loss.

11. The method of claim 1, wherein the biomarker panel comprises regions within and surrounding GJB2, STRC, STRCP1, CATSPER2, OTOA, and OTOAP1.

12. The method of claim 1, wherein the comparison identifies copy-number variations present in the patient dataset.

13. The method of claim 1, wherein the biomarker panel comprises biomarkers associated with copy number variants associated hearing loss.

14. The method of claim 13, wherein the biomarkers associated with copy number variants associated with hearing loss are selected from the group consisting of at least 170 target regions comprising 150-200 base pairs each (FIG. 4).

15. The method of claim 1, wherein the biomarker panel comprises biomarkers associated with infection-induced hearing loss.

16. The method of claim 15, wherein the infection-induced hearing loss is cytomegalovirus-induced hearing loss.

17. The method of claim 15, wherein the biomarkers associated with infection-induced hearing loss are selected from the group consisting of at least six amplicons within highly conserved regions of viral genomes (FIG. 5).

18. (canceled)

19. A method for evaluating hearing loss in a patient, the method comprising:
(a) obtaining nucleic acid sequence data from the patient's genome;
(b) processing the sequence data to generate a patient dataset for the patient; and
(c) comparing the patient dataset to hearing loss biomarkers to identify hearing loss biomarkers in the genomic dataset to create a patient report.

20. The method of claim 19, further comprising:
(d) processing the patient dataset to identify copy number variants in the genomic dataset.

21. A method for evaluating hearing loss in a patient, the method comprising:
(a) obtaining nucleic acid sequence data from the patient's genome;
(b) processing the sequence data to generate a patient dataset for the patient; and
(c) processing the patient dataset to identify copy number variants in the patient dataset to create a patient report.

22-38. (canceled)
